(19) United States
(12) Patent Application Publication
(10) Pub. No.: US 2022/0241275 A1
(43) Pub. Date: Aug. 4, 2022

(54) TARGETED TREATMENT OF CANCERS WITH DYSREGULATED FIBROBLAST GROWTH FACTOR RECEPTOR SIGNALING

(71) Applicant: G1 Therapeutics, Inc., Research Triangle Park, NC (US)

(72) Inventors: Jay Copeland Strum, Hillsborough, NC (US); Chloe Whitworth, Research Triangle Park, NC (US); Daniel M. Freed, Cary, NC (US)

(21) Appl. No.: 17/718,052

(22) Filed: Apr. 11, 2022

Related U.S. Application Data
(63) Continuation of application No. PCT/US2020/055146, filed on Oct. 9, 2020.
(60) Provisional application No. 62/913,055, filed on Oct. 9, 2019.

(51) Int. Cl. A61K 31/498; A61K 31/519; A61K 45/06; A61P 35/00

(52) U.S. Cl. CPC A61K 31/498; A61P 35/00; A61K 45/06; A61K 31/519

(57) ABSTRACT

The present invention provides advantageous methods and compositions for treating a host having a cancer with dysregulation of the FGFR signaling pathway, which includes administering an effective amount of a selective CDK4/6 inhibitor described herein in combination or alternation with a fibroblast growth factor receptor inhibitor.

### STATEMENT OF RELATED APPLICATIONS

This application is a continuation of International Patent Application No. PCT/US2020/055146, filed Oct. 9, 2020, which claims benefit of U.S. Provisional Application No. 62/913,055, filed Oct. 9, 2019. The entirety of each of these applications is hereby incorporated by reference for all purposes.

### FIELD OF THE INVENTION

This invention provides compositions for use in treating cancers having dysregulated fibroblast growth factor receptor (FGFR) signaling with a CDK4/6 inhibitor described herein in combination with a fibroblast growth factor receptor inhibitor, for example a selective FGFR-tyrosine kinase inhibitor, wherein the specific combination provides advantageous or synergistic inhibitory activity, delays acquired resistance in the cancer to the inhibitory effects of the FGFR inhibitor, and/or extends the efficacy of the FGFR inhibitor.

### BACKGROUND OF THE INVENTION

Fibroblast growth factor receptors belong to a family of 4 receptor tyrosine kinases (FGFR1-4) and a fifth receptor (FGFR5) lacking a tyrosine kinase domain (Hallinan N, Finn S, Cuffe S, Rafee S, O'Byrne K, Gately K. Targeting the fibroblast growth factor receptor family in cancer. Cancer Treat Rev 2016; 46: 51-62, Wesche J, Haglund K, Haugsten E M. Fibroblast growth factors and their receptors in cancer. Biochem J 2011; 437: 199-213). FGFRs have been demonstrated to regulate a number of key processes, such as cell migration, proliferation, differentiation, and survival, particularly during embryonic development and in the adult organism during inflammation and wound healing (Hallinan N, Finn S, Cuffe S, Rafee S, O'Byrne K, Gately K. Targeting the fibroblast growth factor receptor family in cancer. Cancer Treat Rev 2016; 46: 51-62, Wesche J, Haglund K, Haugsten E M. Fibroblast growth factors and their receptors in cancer. Biochem J 2011; 437: 199-213). FGFR activity is controlled by a family of FGF ligands, comprised of 22 FGF members (Touat M, Ileana E, Postel-Vinay S, Andre F, Soria J C. Targeting FGFR Signaling in Cancer. Clin Cancer Res 2015; 21: 2684-94) that regulate FGFR tyrosine kinase activity in an autocrine or paracrine tissue-dependent context (Itoh N, Ornitz D M. Functional evolutionary history of the mouse Fgf gene family. Dev Dyn 2008; 237: 18-27). Cancers such as breast, lung, gastric, urothelial and liver cancers such as intrahepatic cholangiocarcinoma and hepatocellular carcinoma harbor hyperactivation of FGFR signaling pathways due to oncogenic aberrations of FGFR family members or hyperactivation of FGFRs due to FGF overproduction, although the nature of the oncogenic alteration may be different between each cancer type.

The fibroblast growth factor receptor (FGFR) is a membrane bound protein that regulates cellular functions including cell proliferation, cell survival, differentiation and migration (Brooks A N, Kilgour E, Smith P D. Molecular pathways: fibroblast growth factor signaling: a new therapeutic opportunity in cancer. Clin Cancer Res 2012; 18: 1855-62). Activation of the FGFR family (FGFR1, FGFR2, FGFR3 and FGFR4) leads to increased downstream activation of oncogenic pathways such as MAPK and AKT (Babina I S, Turner N C. Advances and challenges in targeting FGFR signaling in cancer. Nat Rev Cancer 2017; 17: 318-32). Amplifications, mutations and aberrant fusions of the FGFR gene lead to constitutively activated downstream signaling of these pathways with enhanced cellular growth and migration (Dienstmann R, Patnaik A, Garcia-Carbonero R, Cervantes A, Benavent M, et al. Safety and activity of the first-in-class Sym004 anti-EGFR antibody mixture in patients with refractory colorectal cancer. Cancer Discov 2015; 5: 598-609). In addition, hyperactivation of FGFRs due to FGF overproduction of amplification from cancer and stromal cells has been implicated in aberrant FGFR signaling (see, e.g., Zhang et al., Targeting the Oncogenic FGF-FGFR Axis in Gastric Carcinogenesis. Cells 2019, 8, 637; doi:10.3390/cells8060637).

Emerging clinical data with multiple FGFR inhibitors have validated FGFR as a potential target for anticancer therapeutics. The first FGFR inhibitors to be assessed in the clinic were nonselective FGFR inhibitors, such as brivanib, dovitinib and ponatinib, of which both on-target and off-target activities likely contributed to clinical responses. More recently, FGFR-selective inhibitors, such as infigratinib (BGJ398), have exhibited encouraging antitumor activity in clinical trials. Indeed, six confirmed partial responses were observed with infigratinib at doses ≥100 mg in patients with FGFR1-amplified squamous NSCLC and FGFR3-mutant urothelial cancer patients in a phase I dose escalation trial (Isaacs, Randi and Chen, Xueying and Graus Porta, Diana and Parker, Katie and Yu, Kun and Porter, Dale (2018) Efficacy of BGJ398, a fibroblast growth factor receptor (FGFR) 1-3 inhibitor, in patients with previously treated advanced urothelial carcinoma with FGFR3 alterations. Cancer discovery. ISSN 2159-8290; 2159-8274).

Additional orally available, selective pan-FGFR inhibitors have been described and include: derazantinib (ARQ-087, Arqule) (Hall T G, Yu Y, Eathiraj S, Wang Y, Savage R E, et al. Preclinical activity of ARQ 087, a novel inhibitor targeting FGFR dysregulation. PLoS One 2016; 11: e0162594); AZD4547 (AstraZeneca) (Gavine P R, Mooney L, Kilgour E, Thomas A P, Al-Kadhimi K, et al. AZD4547: an orally bioavailable, potent, and selective inhibitor of the fibroblast growth factor receptor tyrosine kinase family. Cancer Res 2012; 72: 2045-56); infigratinib (BGJ398, Novartis) (Guagnano V, Kauffmann A, Wöhrle S, Stamm C, Ito M, et al. FGFR genetic alterations predict for sensitivity to NVP-BGJ398, a selective Pan-FGFR inhibitor. Cancer Discov 2012; 2: 1118-33); erdafitinib (JNJ-42756493, Janssen) (Perera T P S, Jovcheva E, Mevellec L, Vialard J, De Lange D, et al. Discovery and pharmacological characterization of JNJ-42756493 (Erdafitinib), a functionally selective small-molecule FGFR family inhibitor. Mol Cancer Ther 2017; 16: 1010-20); futibatinib (TAS-120; Taiho) (Kalyukina M, Yosaatmadja Y, Middleditch M J, Patterson A V, Smaill J B, et al. TAS-120 cancer target binding: defining reactivity and revealing the first fibroblast growth factor receptor 1 (FGFR1) irreversible structure. ChemMedChem 2019; 14: 494-500); and pemigatinib (INCB054828, InCyte) (Hollebecque A, Lihou C, Zhen H, Abou-Alfa G K, Borad M, et al. Interim results of fight-202, a phase II, open-label, multicenter study of INCB054828 in patients (pts) with previously treated advanced/metastatic or surgically unresectable cholangiocarcinoma (CCA) with/without fibroblast growth factor (FGF)/FGF receptor (FGFR) genetic alterations. Ann Oncol 2018; 29). These targeted and selective FGFR inhibitors are capable of targeting with specificity the kinase domain of the activated FGFR protein, and have evolved from preclinical testing to early phase clinical trials. Anti-tumor activity in initial clinical trials of urothelial carcinoma and intrahepatic cholangiocarcinoma having FGFR dysregulated pathway signaling has led to larger confirmatory clinical trials, and regulatory approvals.

For example, erdafitinib (BALVERSA; Janssen Biotech) is a selective and potent pan FGFR 1-4 inhibitor that binds to and inhibits enzymatic activity of FGFR1, FGFR2, FGFR3 and FGFR4 based on in vitro data. Erdafitinib has been shown to inhibit FGFR phosphorylation and signaling and decrease cell viability in cell lines expressing FGFR genetic alterations, including point mutations, amplifications, and fusions. Erdafitinib has also demonstrated antitumor activity in FGFR-expressing cell lines and xenograft models derived from tumor types, including bladder cancer.

Erdafitinib has recently been approved for the treatment of adult patients with locally advanced or metastatic urothelial carcinoma (mUC), that has: 1) susceptible FGFR3 or FGFR2 genetic alterations, and 2) progressed during or following at least one line of prior platinum-containing chemotherapy, including within 12 months of neoadjuvant or adjuvant platinum-containing chemotherapy.

Pemigatinib (PEMAZYRE; Incyte Corp.) has recently been approved for the treatment of adult patients with previously treated, unresectable locally advanced or metastatic cholangiocarcinoma with a fibroblast growth factor receptor 2 (FGFR2) fusion or other rearrangement. Clinical trials indicated an overall response rate for pemigatinib monotherapy of 36%, and median duration of response of 9.1 months.

Despite the clinical benefits of these FGFR targeted therapeutics, chronic exposure to the drug results in the potential acquisition of resistance. This secondary refractoriness typically occurs as a result of the accumulation of novel genetic alterations in the kinase target, in other receptor tyrosine kinases (RTKs), or in molecules acting downstream of these RTKs (Camidge D R, Pao W, Sequist L V; Acquired resistance to TKIs in solid tumors: learning from lung cancer. Nat Rev Clin Oncol. 2014 August; 11(8):473-81; Lau et al., Mechanisms of acquired resistance to fibroblast growth factor receptor targeted therapy. Cancer Drug Resist 2019; 2:568-579). The acquired genetic alterations can originate de novo or as clonal expansions of pre-existing low-abundance clones in the tumor. The mechanisms of FGFR resistance are diverse and include the activation of alternate receptor tyrosine kinases, induction of alternate cellular signaling pathways, induction of epithelial-mesenchymal transition, and emergence of gatekeeper mutations such as an FGFR1 V561M substitution, FGFR2 V565I, N550K, or V564 substitution, and FGFR3 V555M substitution (see, e.g., Zhou et al., FGF/FGFR signaling pathway involved resistance in various cancer types. J Cancer. 2020; 11(8): 2000-2007). The development of resistance to the inhibitory effects of FGFR inhibitors has limited their usefulness and ability to promote inhibition for extended periods of time.

It is an object of the present invention to provide compositions, uses, combinations and processes for their manufacture of medicaments which effectively target cancers with dysregulation of FGFR signaling pathway due to FGFR or FGF aberrations and effectively reduce or delay the development of acquired resistance to FGFR inhibitors that target dysregulation of FGFR signaling with a therapeutic regime capable of long-term administration.

### SUMMARY OF THE INVENTION

The present invention provides advantageous compositions and uses for the administration to a host, for example a human, having a cancer with dysregulation of the FGFR signaling pathway caused by an FGFR family member aberration or FGF aberration, which includes an effective amount of the described selective CDK4/6 inhibitors described herein in combination or alternation with an effective amount of a fibroblast growth factor receptor (FGFR) inhibitor, for example a selective FGFR-tyrosine kinase inhibitor (TKI). The administration of a selective FGFR inhibitor in combination or alternation with the administration of the described selective CDK 4/6 inhibitor provides significant advantageous and in certain cases synergistic inhibition of tumor growth and progression, which increases therapeutic effectiveness and may reduce or delay the acquisition of acquired resistance (see, for example, FIGS. 1A, 1B, and 1C, and FIG. 2, and Examples described below). By incorporating the selective CDK4/6 inhibitors described herein into a therapeutic regime with a FGFR inhibitor, for example a selective FGFR-TKI, the select combinations provide efficacious anti-cancer treatments capable of extended cancer proliferation suppression and administration with limited toxicity stacking contributed by the CDK4/6 inhibitor.

It is known that while FGFR inhibitors such as FGFR-TKIs are beneficial treatments for suitable cancer patients harboring FGFR aberrations, their long-term use is associated with the potential development of an acquired resistance to their FGFR inhibitory effects by the cancer being treated. Furthermore, FGFR-TKI use is associated with a high incidence of side effects that can be difficult to manage. For example, common adverse events associated with nonselective FGFR-TKIs include fatigue, anorexia, pyrexia, gastrointestinal disorders, athralgia, liver toxicity, hypertension, proteinuria, thrombotic microangiopathy, and hyperthyroidism. Common adverse events for selective FGFR-TKIs include hyperphosphatemia, alopecia, mucosal dryness, dysgeusia, mucositis, dry eye, onycholysis, diarrhea, conjunctivitis, keratitis, osteoarticular pains, myalgias, and muscle cramps. The high incidence of side effects makes the use of additional anti-cancer agents in combination with FGFR inhibitors challenging. The compositions and uses of the present invention provide synergistic inhibition while combating the development of FGFR inhibitor resistance without significantly increasing the associated side effects with FGFR inhibitor use by using a highly selective, transient CDK4/6 inhibitor described herein.

In some embodiments, the CDK4/6 inhibitors described herein can be administered in combination with an FGFR inhibitor in a manner that allows daily administration of the CDK4/6 inhibitor and FGFR inhibitor to the host, wherein the CDK4/6 inhibitor, or the CDK4/6 inhibitor and FGFR inhibitor, are administered daily without a drug holiday or without serious side-effect stacking problems, for example severe dose limiting gastrointestinal issues or neutropenia such as that seen with other CDK4/6 class inhibitors such as, for example palbociclib which has been approved for the treatment of ER+, HER2− metastatic breast cancer but due to its associated myelosuppressive side-effects, requires a dosing holiday. The CDK4/6 inhibitors described for the compositions and uses herein in combination with an FGFR inhibitor are short-acting, having short half-lives (less than about 18 hours) and limited side-effects, thus allowing their inclusion in a long-term treatment regime without the need for treatment holidays due to use of the CDK4/6 inhibitor. Furthermore, by using these particular CDK4/6 inhibitors, therapy-limiting side effects such as neutropenia and gastrointestinal complications associated with other CDK4/6 inhibitors are avoided, and potential treatment limiting side-effect stacking associated with combining a CDK4/6 inhibitor with an FGFR inhibitor in a combination treatment can be significantly reduced. The CDK4/6 inhibitors described herein are particularly useful in therapeutic regimens requiring long-term treatment, as required for FGFR inhibitor treatment in, for example gastric adenocarcinoma, non-small cell lung cancer, breast cancer, and hepatocellular carcinoma and intrahepatic cholangiocarcinoma, while minimizing the effect of CDK4/6 inhibitory toxicity on CDK4/6 replication dependent healthy cells, such as hematopoietic stem cells and hematopoietic progenitor cells (together referred to as HSPCs).

Due to the reduced risk of side effects associated with the use of the CDK4/6 inhibitors described herein in the treatment of abnormal cellular proliferation such as cancer with dysregulated fibroblast growth factor receptor (FGFR) signaling, prolonged continuous, daily dosing, for example, 14 days or more, 21 days or more, 24 days or more, 28 days or more, 35 days or more, 42 days or more, 84 days or more, 168 days or more, of the CDK4/6 inhibitor, or the CDK4/6 inhibitor and FGFR inhibitor can be accomplished. In an alternative embodiment, the FGFR inhibitor is administered at a set schedule, for example once every three weeks, once a week, every day for 5 days of a 7-day cycle, every day for 14 days of a 21-day cycle, or every day for 21 days of a 28-day cycle, or every day for 28 days of a 28-day cycle, and the CDK4/6 inhibitor is administered every day during the entirety of the cycle. In some embodiments, the CDK4/6 inhibitor is administered twice a day. In a further alternative embodiment, the FGFR inhibitor is administered for a prolonged continuous daily dosing period, for example, 14 days or more, 21 days or more, 24 days or more, 28 days or more, 35 days or more, 42 days or more, 84 days or more, 168 days or more, and a CDK4/6 inhibitor described herein is administered intermittently, for example, at least once a week, at least once every ten days, at least once every two weeks, at least once every three weeks, or at least once a month. In still another alternative embodiment, the CDK4/6 inhibitor described herein is administered at least on the same administration schedule as the FGFR inhibitor.

The CDK4/6 inhibitors for use in the compositions and treatments described herein are selective, short acting CDK4/6 inhibitors selected from:

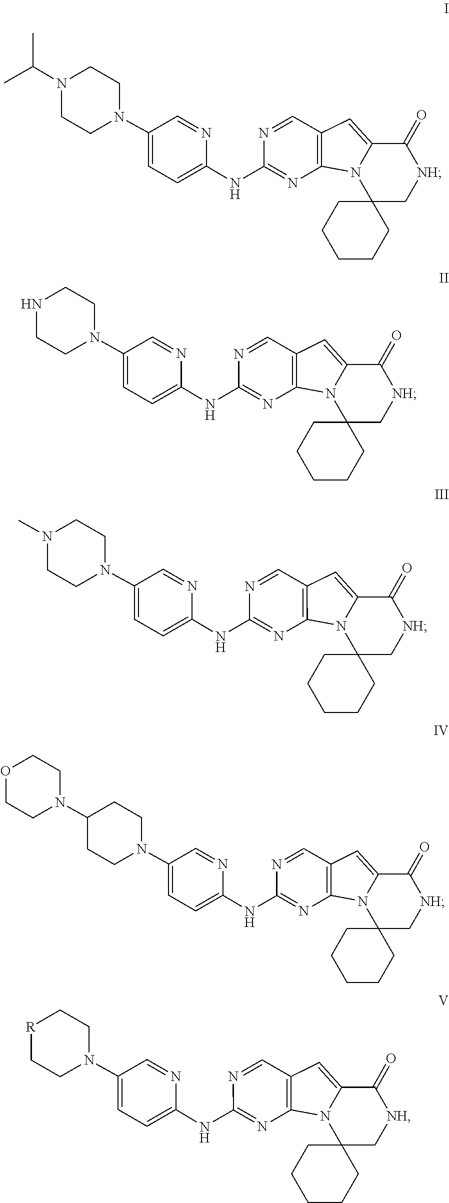

wherein R is C(H)X, NX, C(H)Y, or C(X)₂,

where X is hydrogen, straight, branched or cyclic C₁ to C₅ alkyl group, including methyl, ethyl, propyl, cyclopropyl, isopropyl, butyl, sec-butyl, tert-butyl, isobutyl, cyclobutyl, pentyl, isopentyl, neopentyl, tert-pentyl, sec-pentyl, and cyclopentyl; and

Y is NR₁R₂ wherein R₁ and R₂ are independently X, or wherein R₁ and R₂ are alkyl groups that together form a bridge that includes one or two heteroatoms (N, O, or S);

and wherein two X groups can together form an alkyl bridge or a bridge that includes one or two heteroatoms (N, S, or O) to form a spiro compound, or

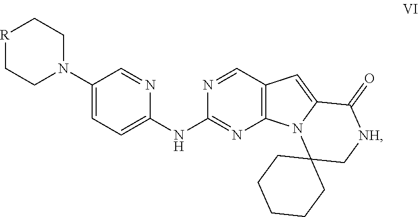

wherein R is NX and wherein X is hydrogen, isopropyl, or methyl;

or a pharmaceutically acceptable salt, isotopic analog, or prodrug thereof, optionally in a pharmaceutically acceptable carrier to form a composition. Compounds I-VI are described in, for example, US 2013/0237544, incorporated by reference herein.

Compound I, also known as “lerociclib” and developed by G1 Therapeutics, Inc., has been investigated in a number of human clinical trials as an antineoplastic agent to treat 1) EGFR-mutant non-small cell lung carcinoma in combination with the EGFR inhibitor osimertinib (Tagrisso®), and 2) ER+, HER2− breast cancer in combination with fulvestrant.

Compound III, also known as “trilaciclib” and developed by G1 Therapeutics, Inc., is currently being investigated in a number of human clinical trials for use as a myelopreservation agent administered via intravenous injection before chemotherapy with 1) gemcitabine and carboplatin in metastatic triple negative breast cancer (mTNBC), 2) topotecan in advanced staged small cell lung carcinoma (SCLC), 3) carboplatin and etoposide in SCLC, and 4) carboplatin, etoposide, and the PD-L1 immune checkpoint inhibitor atezolizumab (Tecentriq®) in SCLC.

Cancers with dysregulation of the FGFR signaling pathway that can be treated using the compositions and treatments described herein include, but are not limited to, liver cancer including hepatocellular carcinoma and intrahepatic cholangiocarcinoma, gastroesophageal cancer, endometrial cancer, ovarian cancer, gastric cancer including gastric adenocarcinoma, gliomas including glioblastoma, head and neck cancer, breast cancer, including ER+/HER2+ breast cancer, non-small cell lung cancer (NSCLC), including squamous cell lung cancer and large cell lung cancer, pilocytic astrocytoma, and rhabdomyosarcoma, as well as other cancers as described herein that may be susceptible to FGFR inhibition due to an FGFR or FGF aberration. Cancers that can be treated using the compositions and treatments described include those with a dysregulation of the FGFR signaling pathway leading to aberrant proliferation, which may occur through, but not limited to, FGFR gene amplification, FGFR overexpression, FGFR fusions of FGFR translocations, FGFR point mutations, and FGFR gene rearrangements, or other FGFR activating molecular alterations, or FGF aberrations, such as FGF overexpression or amplification. In some embodiments, the cancer is not urothelial cancer. In some embodiments, the cancer is advanced or metastatic.

In one aspect, the FGFR inhibitors for use in the present invention in combination or alternation with the CDK4/6 inhibitors described herein are selective FGFR inhibitors, for example, selective FGFR-tyrosine kinase inhibitors (TKIs). In alternative embodiments, the FGFR inhibitor for administration can be selected from non-selective FGFR inhibitors, selective FGFR monoclonal antibodies, and FGF traps.

In particular aspects, the CDK4/6 inhibitors described herein are administered to a host with a cancer having an aberrant FGFR signaling pathway in combination with a selective FGFR inhibitor. Selective FGFR inhibitors generally inhibit FGFR signaling activity preferentially over inhibition of other targets, although inhibition of other targets may occur to a lesser extent. Selective FGFR inhibitors for use in the methods described herein include, but are not limited to, erdafitinib (Janssen, BALVERSA), infigratinib (BGJ398, QED Therapeutics), pemigatinib (PEMAZYRE; INCB54828, Incyte), AZD4547 (AstraZeneca), futibatinib (TAS-120; Taiho Pharmaceuticals), derazantinib (Arqule, ARQ087), roblitinib (FGF-401, Novartis), LY287445 (Eli Lilly), INCB062079 (Incyte) BLU9931 (Blueprint Medicines), PRN1371 (Principia Biopharma), PD173074 (Pfizer), Debio1347 (Debiopharm), fisogatinib, H3B-6527, bemarituzumab, alofanib, MGFR1877S, vofatamab, and FIIN-2. In some embodiments, a CDK4/6 inhibitor selected from Compounds I-VI is administered in combination or alternation with erdafitinib. In some embodiments, a CDK4/6 inhibitor selected from Compounds I-VI is administered in combination or alternation with infigratinib. In some embodiments, a CDK4/6 inhibitor selected from Compounds I-VI is administered in combination or alternation with pemigatinib. In some embodiments, a CDK4/6 inhibitor selected from Compounds I-VI is administered in combination or alternation with AZD4547. In some embodiments, a CDK4/6 inhibitor selected from Compounds I-VI is administered in combination or alternation with futibatinib (TAS-120). In some embodiments, a CDK4/6 inhibitor selected from Compounds I-VI is administered in combination or alternation with derazatanib. In some embodiments, a CDK4/6 inhibitor selected from Compounds I-VI is administered in combination or alternation with roblitinib. In some embodiments, a CDK4/6 inhibitor selected from Compounds I-VI is administered in combination or alternation with LY287445. In some embodiments, a CDK4/6 inhibitor selected from Compounds I-VI is administered in combination or alternation with INCB062079. In some embodiments, a CDK4/6 inhibitor selected from Compounds I-VI is administered in combination or alternation with BLU9931. In some embodiments, a CDK4/6 inhibitor selected from Compounds I-VI is administered in combination or alternation with PRN1371. In some embodiments, a CDK4/6 inhibitor selected from Compounds I-VI is administered in combination or alternation with PD1733074. In some embodiments, a CDK4/6 inhibitor selected from Compounds I-VI is administered in combination or alternation with Debio1347. In some embodiments, a CDK4/6 inhibitor selected from Compounds I-VI is administered in combination or alternation with FIIN-2. In some embodiments, a CDK4/6 inhibitor selected from Compounds I-VI is administered in combination or alternation with H3B-6527. In some embodiments, a CDK4/6 inhibitor selected from Compounds I-VI is administered in combination or alternation with fisogatinib. In some embodiments, a CDK4/6 inhibitor selected from Compounds I-VI is administered in combination or alternation with alofanib. In some embodiments, a CDK4/6 inhibitor selected from Compounds I-VI is administered in combination or alternation with MGFR1877S. In some embodiments, a CDK4/6 inhibitor selected from Compounds I-VI is administered in combination or alternation with vofatamab. selected from Compounds I-VI is administered in combination or alternation with infigratinib. In some embodiments, a CDK4/6 inhibitor selected from Compounds I-VI is administered in combination or alternation with bemarituzumab. In some embodiments, the selective CDK4/6 inhibitor administered is Compound VI. In some embodiments, the CDK4/6 inhibitor is Compound I, Compound IA, or Compound IA, Form B. In alternative embodiments, the CDK4/6 inhibitor is Compound III. In some embodiments, the cancer is an FGFR aberrant non-small cell lung cancer. In some embodiments, the non-small cell lung cancer is squamous cell lung cancer. In some embodiments, the non-small cell lung cancer is large cell lung cancer. In some embodiments, the cancer is FGFR aberrant gastric adenocarcinoma.

In one aspect, the present invention provides compositions and treatments for treating a patient with a cancer having dysregulation of the FGFR signaling pathway due to an FGFR family member aberration wherein the treatment comprises administering to the patient an effective amount of a selective CDK4/6 inhibitor described herein in combination with an effective amount of an FGFR inhibitor such as a selective FGFR-TKI, wherein the administration of the CDK4/6 inhibitor enhances anti-cancer activity and/or delays resistance to an FGFR inhibitor in the cancer. In particular, the use of the selective CDK4/6 inhibitors described herein in combination with an FGFR inhibitor may be efficacious in delaying the onset of acquired resistance or reducing acquired resistance to the administered FGFR inhibitor. Accordingly, the compositions and their use in treatments described herein can expand the length of time a cancer is responsive to FGFR inhibitor treatment.

In one aspect of the invention, provided herein are compositions and treatments for treating a patient with a cancer with dysregulation of FGFR signaling wherein the treatment comprises administering a therapeutically effective amount of a selective CDK4/6 inhibitor described herein in combination with an effective amount of an FGFR inhibitor, wherein the patient is FGFR inhibitor and CDK4/6 inhibitor treatment naïve. In some embodiments, the selective CDK4/6 inhibitor administered is Compound VI. In some embodiments, the selective CDK4/6 inhibitor is Compound I, Compound IA, or Compound IA, Form B. In some embodiments, the selective CDK4/6 inhibitor is Compound III. In some embodiments, the FGFR inhibitor is a selective FGFR inhibitor. In some embodiments, the selective FGFR inhibitor is selected from erdafitinib, infigratinib, pemigatinib, AZD4547, futibatinib, derazantinib, roblitinib, LY287445, INCB062079, PD173074, FIIN-2, fisogatinib, H3B-6527, alofanib, MGFR1877S, vofatamab, bemarituzumab, or Debio1347. In some embodiments, the FGFR inhibitor is erdafitinib. In some embodiments, the FGFR inhibitor is infigratinib. In some embodiments, the FGFR inhibitor is pemigatinib. In some embodiments, the FGFR inhibitor is AZD4547. In some embodiments, the FGFR inhibitor is futibatinib. In some embodiments, the FGFR inhibitor is derazantinib. In some embodiments, the FGFR inhibitor is roblatinib. In some embodiments, the FGFR inhibitor is LY287445. In some embodiments, the FGFR inhibitor is INCB062079. In some embodiments, the FGFR inhibitor is Debio1347. In some embodiments, the FGFR inhibitor is FIIN-2. In some embodiments, the FGFR inhibitor is fisogatinib. In some embodiments, the FGFR inhibitor is H3B-6527. In some embodiments, the FGFR inhibitor is BLU9931. In some embodiments, the FGFR inhibitor is PRN1371. In some embodiments, a CDK4/6 inhibitor selected from Compounds I-VI is administered in combination or alternation with alofanib. In some embodiments, a CDK4/6 inhibitor selected from Compounds I-VI is administered in combination or alternation with MGFR1877S. In some embodiments, a CDK4/6 inhibitor selected from Compounds I-VI is administered in combination or alternation with vofatamab. In some embodiments, a CDK4/6 inhibitor selected from Compounds I-VI is administered in combination or alternation with bemarituzumab. In some embodiments, the cancer is an FGFR aberrant non-small cell lung cancer. In some embodiments, the non-small cell lung cancer is squamous cell lung cancer. In some embodiments, the non-small cell lung cancer is large cell lung cancer. In some embodiments, the cancer is FGFR aberrant gastric adenocarcinoma.

In one aspect of the invention, provided herein are compositions and treatments for treating a patient with a cancer with dysregulation of FGFR signaling wherein the treatment comprises administering a therapeutically effective amount of a selective CDK4/6 inhibitor described herein in combination with an effective amount of an FGFR inhibitor, wherein the patient is CDK4/6 inhibitor treatment naïve. In some embodiments, the selective CDK4/6 inhibitor administered is Compound VI. In some embodiments, the selective CDK4/6 inhibitor is Compound I, Compound IA, or Compound IA, Form B. In an alternative embodiment, the selective CDK4/6 inhibitor is Compound III. In some embodiments, the FGFR-TKI is selected from erdafitinib, infigratinib, pemigatinib, AZD4547, futibatinib (TAS-120), derazantinib, roblitinib, LY287445, INCB062079, FIIN-2, fisogatinib, H3B-6527, alofanib, MGFR1877S, vofatamab, bemarituzumab, or Debio1347. In some embodiments, the FGFR inhibitor is erdafitinib. In some embodiments, the FGFR inhibitor is infigratinib. In some embodiments, the FGFR inhibitor is pemigatinib. In some embodiments, the FGFR inhibitor is AZD4547. In some embodiments, the FGFR inhibitor is futibatinib. In some embodiments, the FGFR inhibitor is derazantinib. In some embodiments, the FGFR inhibitor is roblatinib. In some embodiments, the FGFR inhibitor is LY287445. In some embodiments, the FGFR inhibitor is INCB062079. In some embodiments, the FGFR inhibitor is Debio1347. In some embodiments, the FGFR inhibitor is FIIN-2. In some embodiments, the FGFR inhibitor is fisogatinib. In some embodiments, the FGFR inhibitor is H3B-6527. In some embodiments, the FGFR inhibitor is BLU9931. In some embodiments, the FGFR inhibitor is PRN1371. In some embodiments, the FGFR inhibitor is PD173074. In some embodiments, a CDK4/6 inhibitor selected from Compounds I-VI is administered in combination or alternation with alofanib. In some embodiments, a CDK4/6 inhibitor selected from Compounds I-VI is administered in combination or alternation with MGFR1877S. In some embodiments, a CDK4/6 inhibitor selected from Compounds I-VI is administered in combination or alternation with vofatamab. In some embodiments, a CDK4/6 inhibitor selected from Compounds I-VI is administered in combination or alternation with bemarituzumab. In some embodiments, the cancer is an FGFR aberrant non-small cell lung cancer. In some embodiments, the non-small cell lung cancer is squamous cell lung cancer. In some embodiments, the non-small cell lung cancer is large cell lung cancer. In some embodiments, the cancer is FGFR aberrant gastric adenocarcinoma.

In one alternative aspect, provided herein are compositions and treatments for treating a host with a cancer with a dysregulated FGFR signaling pathway, wherein the treatment includes:

a) confirming the presence of an FGFR aberration in the cancer;

b) administering to the patient an effective amount of a selective FGFR inhibitor; and,

c) administering to the patient an effective amount of a selective CDK 4/6 inhibitor described herein in combination with the FGFR inhibitor. In some embodiments, the selective CDK4/6 inhibitor administered is Compound VI. In some embodiments, the selective CDK4/6 inhibitor administered is Compound I, Compound IA, or Compound IA, Form B. In an alternative embodiment, the selective CDK4/6 inhibitor administered is Compound III. In some embodiments, the FGFR inhibitor is a selective FGFR inhibitor. In some embodiments, the FGFR-TKI is selected from erdafitinib, infigratinib, pemigatinib, AZD4547, futibatinib (TAS-120), derazantinib, roblitinib, LY287445, INCB062079, FIIN-2, fisogatinib, H3B-6527, alofanib, MGFR1877S, vofatamab, bemarituzumab or Debio1347. In some embodiments, the FGFR inhibitor is erdafitinib. In some embodiments, the FGFR inhibitor is infigratinib. In some embodiments, the FGFR inhibitor is pemigatinib. In some embodiments, the FGFR inhibitor is AZD4547. In some embodiments, the FGFR inhibitor is futibatinib. In some embodiments, the FGFR inhibitor is derazantinib. In some embodiments, the FGFR inhibitor is roblatinib. In some embodiments, the FGFR inhibitor is LY287445. In some embodiments, the FGFR inhibitor is INCB062079. In some embodiments, the FGFR inhibitor is Debio1347. In some embodiments, the FGFR inhibitor is FIIN-2. In some embodiments, the FGFR inhibitor is fisogatinib. In some embodiments, the FGFR inhibitor is H3B-6527. In some embodiments, the FGFR inhibitor is BLU9931. In some embodiments, the FGFR inhibitor is PRN1371. In some embodiments, the FGFR inhibitor is PD173074. In some embodiments, a CDK4/6 inhibitor selected from Compounds I-VI is administered in combination or alternation with alofanib. In some embodiments, a CDK4/6 inhibitor selected from Compounds I-VI is administered in combination or alternation with MGFR1877S. In some embodiments, a CDK4/6 inhibitor selected from Compounds I-VI is administered in combination or alternation with vofatamab. In some embodiments, the FGFR inhibitor is AZD4547. In some embodiments, the FGFR inhibitor is bemarituzumab. In some embodiments, the cancer is an FGFR aberrant non-small cell lung cancer. In some embodiments, the non-small cell lung cancer is squamous cell lung cancer. In some embodiments, the non-small cell lung cancer is large cell lung cancer.

In one alternative aspect, provided herein are compositions and treatments for treating a host with a cancer with a dysregulated FGFR signaling pathway, wherein the treatment includes:

a) administering to the patient an FGFR inhibitor;

b) monitoring the patient's FGFR-aberration status; and,

c) administering to the patient a selective CDK 4/6 inhibitor described herein in combination with the FGFR inhibitor upon the detection of an FGFR aberration or mutation or non-FGFR mutation that confers resistance upon the cancer to the inhibitory effects of the FGFR inhibitor. In some embodiments, the FGFR aberration is an FGFR1 V561M substitution, an FGFR2 V565I mutation, an FGFR2 N550K mutation, an FGFR2 V564 mutation, or an FGFR3 V555M mutation. In some embodiments, the selective CDK4/6 inhibitor administered is Compound I, Compound IA, or Compound IA, Form B. In some embodiments, the selective CDK4/6 inhibitor administered is Compound III. In some embodiments, the FGFR inhibitor is infigratinib. In some embodiments, the FGFR inhibitor is pemigatinib. In some embodiments, the FGFR inhibitor is AZD4547. In some embodiments, the FGFR inhibitor is futibatinib. In some embodiments, the FGFR inhibitor is derazantinib. In some embodiments, the FGFR inhibitor is roblatinib. In some embodiments, the FGFR inhibitor is LY287445. In some embodiments, the FGFR inhibitor is INCB062079. In some embodiments, the FGFR inhibitor is alofanib. In some embodiments, the FGFR inhibitor is bemarituzumab. In some embodiments, a CDK4/6 inhibitor selected from Compounds I-VI is administered in combination or alternation with MGFR1877S. In some embodiments, a CDK4/6 inhibitor selected from Compounds I-VI is administered in combination or alternation with vofatamab. In some embodiments, the FGFR inhibitor is Debio1347. In some embodiments, the FGFR inhibitor is FIIN-2. In some embodiments, the FGFR inhibitor is fisogatinib. In some embodiments, the FGFR inhibitor is H3B-6527. In some embodiments, the FGFR inhibitor is BLU9931. In some embodiments, the FGFR inhibitor is PRN1371. In some embodiments, the FGFR inhibitor is PD173074. In some embodiments, the cancer is an FGFR aberrant non-small cell lung cancer. In some embodiments, the non-small cell lung cancer is squamous cell lung cancer. In some embodiments, the non-small cell lung cancer is large cell lung cancer. In some embodiments, the cancer is FGFR aberrant gastric adenocarcinoma.

The administration regime for use in the present invention may include daily dosing of both the FGFR inhibitor and the CDK 4/6 inhibitor. For example, the FGFR inhibitor may be administered at least once a day with the CDK 4/6 inhibitor. Alternatively, the FGFR inhibitor may be administered as least once a day and the CDK 4/6 inhibitor dosed at least once a day, for example once a day, twice a day, or three times a day. Because the CDK 4/6 inhibitors described herein are highly tolerable, the therapeutic regime can be dosed continuously for an extended period without the need for a drug holiday, further extending the combinations beneficial effects. Accordingly, provided herein are compositions and treatments for treating a cancer harboring an FGFR or FGF aberration, wherein the treatment includes administering a CDK 4/6 inhibitor described herein in combination with a FGFR inhibitor, wherein the combination is administered continuously, for example at least 14 days, at least 21 days, at least 28 days, at least 35 days, at least 56 days, at least 70 days, at least 102 days, at least 204 days, or more, without the need for a scheduled drug holiday. In some embodiments, the oral dosing regime comprises about 200 mg, 300 mg, 400 mg, 500 mg, or 650 mg of CDK 4/6 inhibitor that is dosed once a day. In some embodiments, the oral dosing regime comprises about 100 mg, 150 mg, or 200 mg of CDK 4/6 inhibitor that is dosed twice a day, optionally spaced about 12 hours apart. In some embodiments, the CDK 4/6 inhibitor is Compound I, Compound IA, or Compound IA, Form B and is administered as an oral dose of 150 mg twice a day. In some embodiments, the CDK 4/6 inhibitor is Compound III

In alternative embodiments, wherein the standard dosing regimen of the FGFR inhibitor is a set administration with a scheduled drug holiday, for example daily administration for the first 5 days of a 7-day cycle, first 14 days of a 21-day cycle, or first 21 days of a 28-day cycle, the CDK4/6 inhibitor can be administered daily during the period in the cycle for which the FGFR is administered, and when the FGFR is not administered during a period in the cycle (the “off-period”or “holiday”), the CDK4/6 inhibitor can continue to be administered daily for the entirety of the cycle.

### BRIEF DESCRIPTIONS OF THE DRAWINGS

FIG. 1A is a line graph of IC₅₀ curves for H1581 (FGFR1m) NSCLC cells treated with vehicle (DMSO), 300 nM Lerociclib, 300 nM erdafitinib, 300 nM Lerociclib+300 nM erdafitinib, and 300 nM Palbociclib+300 nM erdafitinib. The x-axis is the log[inhibitor] and the y-axis is the relative absorbance compared to the DMSO control.

FIG. 1B is a line graph of IC50 curves for Snu-16 (FGFR2m) gastric cancer cells treated with vehicle (DMSO), 300 nM Lerociclib, 300 nM erdafitinib, 300 nM Lerociclib+300 nM erdafitinib, and 300 nM Palbociclib+300 nM erdafitinib. The x-axis is the log[inhibitor] and the y-axis is the relative absorbance compared to the DMSO control.

FIG. 1C is a line graph of IC₅₀ curves for RT4 (FGFR3m) bladder cancer cells treated with vehicle (DMSO), 300 nM Lerociclib, 300 nM erdafitinib, 300 nM Lerociclib+300 nM erdafitinib, and 300 nM Palbociclib+300 nM erdafitinib. The x-axis is the log[inhibitor] and the y-axis is the relative absorbance compared to the DMSO control.

FIG. 2 is a line graph that shows the absorbance of solubilized crystal violet stain for RT4 (FGFR3m) bladder cancer cells treated with vehicle (DMSO), 300 nM Lerociclib, 100 nM erdafitinib, or 100 nM Lerociclib+300 nM erdafitinib after 7, 13, 18 and 25 days. The x-axis measures time in days. The y-axis measured absorbance at 562 nm.

### DETAILED DESCRIPTION OF THE INVENTION

### Definitions

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individual recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and do not pose a limitation on the scope of the invention unless otherwise claimed. Unless defined otherwise, technical and scientific terms used herein have the same meaning as is commonly understood by one of skill in the art to which this invention belongs.

An “effective amount” as used herein, means an amount which provides a therapeutic or prophylactic benefit.

To “treat” a disease as the term is used herein, means to reduce the frequency or severity of at least one sign or symptom of a disease or disorder experienced by a host (i.e. palliative treatment) or to decrease a cause or effect of the disease or disorder (i.e. disease-modifying treatment).

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

A “dosage form” means a unit of administration of an active agent. Non-limiting dosage forms include tablets, capsules, injections, suspensions, liquids, intravenous fluids, emulsions, creams, ointments, suppositories, inhalable forms, transdermal forms, and the like. In some embodiments, the dosage form is a solid tablet or capsule.

“Parenteral” administration of a compound includes, e.g., subcutaneous (s.c.), intravenous (i.v.), intramuscular (i.m.), or intrasternal injection, or infusion techniques.

As used herein, “pharmaceutical compositions” are compositions comprising at least one active agent, such as a compound or salt of one of the active compounds disclosed herein, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985). Wherein the methods described herein identify the administration of a particular compound, it is understood that administration of the compound's pharmaceutically acceptable salt, if applicable, is encompassed as an embodiment.

As used herein, the term “prodrug” means a compound which when administered to a host in vivo is converted into the parent drug. As used herein, the term “parent drug” means any of the presently described chemical compounds that are useful to treat any of the disorders described herein, or to control or improve the underlying cause or symptoms associated with any physiological or pathological disorder described herein in a host, typically a human. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Nonlimiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

The term “carrier” applied to pharmaceutical compositions/combinations of the invention refers to a diluent, excipient, or vehicle with which an active compound is provided.

The “patient” or “host” treated is typically a human patient, although it is to be understood the methods described herein are effective with respect to other animals, such as mammals. More particularly, the term patient can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like.

“Acquired resistance,” as used herein, refers to a condition wherein a cancer that was or is initially sensitive to the inhibitory effects of an inhibitor compound becomes non-responsive or less-responsive over time to the effects of that compound. Without wishing to be bound by any one theory, it is believed that acquired resistance to an inhibitor occurs due to one or more additional mutations or genetic alterations in bypass signaling that develops after the onset of inhibitor treatment. In certain embodiments, a tumor or cancer that has acquired resistance to an inhibitor is a tumor or cancer who has a cell population wherein less than 50%, 40%, 30% 20%, 15%, 10%, or 5% of its cells experience inhibition, leading to disease progression.

By “off-cycle” or “drug holiday” is meant a time period during which the host is not administered or exposed to the CDK4/6 inhibitor and/or FGFR inhibitor. For example, in a treatment regime wherein the host is administered the CDK4/6 inhibitor and/or FGFR inhibitor for 21 straight days and is not administered the CDK4/6 inhibitor and/or FGFR inhibitor for 7 days, and the regime is repeated a number of times or cycles, the 7 day period of non-administration is considered the “off-cycle” or “drug holiday.” Off-cycle and drug holiday may also refer to an interruption in a treatment regime wherein the host is not administered the CDK4/6 inhibitor and/or FGFR inhibitor for a time due to a deleterious side effect, for example, myelosuppression, diarrhea, or other side-effect requiring a cessation of drug administration.

CDK4/6 Inhibitors

CDK4/6 inhibitors for use in the present invention include Compound I, Compound II, Compound III, Compound IV, and Compound V, or pharmaceutically acceptable salts thereof.

Publications that describe compounds of this general class include the following. WO 2014/144326 filed by Strum et al. and assigned to G1 Therapeutics describes compounds and methods for protection of normal cells during chemotherapy using pyrimidine based CDK4/6 inhibitors. WO 2014/144596 filed by Strum et al. and assigned to G1 Therapeutics describes compounds and methods for protection of hematopoietic stem and progenitor cells against ionizing radiation using pyrimidine based CDK4/6 inhibitors. WO 2014/144847 filed by Strum et al. and assigned to G1 Therapeutics describes HSPC-sparing treatments of abnormal cellular proliferation using pyrimidine based CDK4/6 inhibitors. WO2014/144740 filed by Strum et al. and assigned to G1 Therapeutics describes highly active anti-neoplastic and anti-proliferative pyrimidine based CDK 4/6 inhibitors. WO 2015/161285 filed by Strum et al. and assigned to G1 Therapeutics describes tricyclic pyrimidine based CDK inhibitors for use in radioprotection. WO 2015/161287 filed by Strum et al. and assigned to G1 Therapeutics describes analogous tricyclic pyrimidine based CDK inhibitors for the protection of cells during chemotherapy. WO 2015/161283 filed by Strum et al. and assigned to G1 Therapeutics describes analogous tricyclic pyrimidine based CDK inhibitors for use in HSPC-sparing treatments of RB-positive abnormal cellular proliferation. WO 2015/161288 filed by Strum et al. and assigned to G1 Therapeutics describes analogous tricyclic pyrimidine based CDK inhibitors for use as anti-neoplastic and anti-proliferative agents. WO 2016/040858 filed by Strum et al. and assigned to G1 Therapeutics describes the use of combinations of pyrimidine based CDK4/6 inhibitors with other anti-neoplastic agents. WO 2016/040848 filed by Strum et al. and assigned to G1 Therapeutics describes compounds and methods for treating certain Rb-negative cancers with CDK4/6 inhibitors and topoisomerase inhibitors. WO 2019/136451 filed by Beelen et al. and assigned to G1 Therapeutics describes specific dosage regimes using Compound I for the treatment of cancers. WO 2019/199883 filed by Strum et al. and assigned to G1 Therapeutics describes specific combinations of Compound I and certain tyrosine kinase inhibitors for the treatment of cancers have specific oncogenic driving mutations.

In one aspect, provided herein is a composition for use in treating a host with a dysregulated FGFR cancer wherein the composition is Compound I and the treatment comprises administering to the host an effective amount of Compound I, or a pharmaceutically acceptable salt thereof, and an effective amount of an FGFR inhibitor. Compound I, known as lerociclib (2′-((5-(4-isopropylpiperazin-1-yl) pyridin-2-yl)amino)-7′,8′-dihydro-6′H-spiro[cyclohexane-1,9′-pyrazino[1′,2′:1,5]pyrrolo[2,3-d]pyrimidin]-6′-one), is a highly selective CDK4/6 inhibitor having the structure:

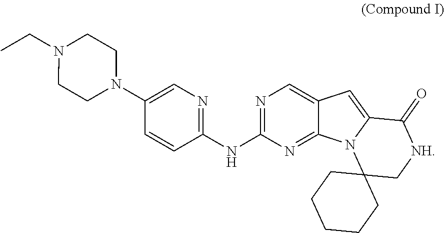 [SMILES: CCN1CCN(c2ccc(Nc3ncc4cc5n(c4n3)C3(CCCCC3)CNC5=O)nc2)CC1]

Lerociclib can be administered orally or intravenously, and was previously described in US 2013-0237544, incorporated herein by reference. Lerociclib can be prepared as previously described in US 2019-0135820, incorporated herein by reference. Lerociclib induces inhibition of cell proliferation in a variety of CDK4/6-dependent tumorigenic cell lines including breast, melanoma, leukemia, and lymphoma cells and inhibits RB phosphorylation in vitro and in vivo. Additional favorable therapeutic properties of lerociclib, including the selectivity for tumors over plasma in mouse xenograft tumors, are highlighted in an article released in a peer reviewed journal (Bisi, et al., Preclinical development of G1T38: A novel, potent and selective inhibitor of cyclin dependent kinases 4/6 for use as an oral antineoplastic in patients with CDK 4/6 sensitive tumors”, Oncotarget, Mar. 15, 2017). See also U.S. Pat. No. 9,527,857.

In some embodiments, Compound I is administered as the di-hydrochloride salt:

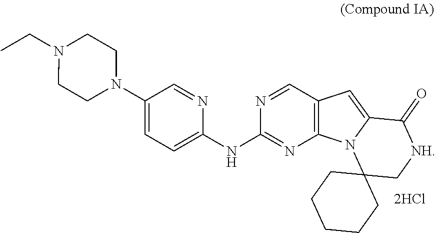 [SMILES: CCN1CCN(c2ccc(Nc3ncc4cc5n(c4n3)C3(CCCCC3)CNC5=O)nc2)CC1]

In some embodiments, Lerociclib is administered as the isolated Form B morphic form of the dihydrochloride salt (Compound IA, Form B), which is characterized by an X-ray powder diffraction (XRPD) pattern comprising at least three 2theta values from 6.5±0.2°, 9.5±0.2°, 14.0±0.2°, 14.4±0.2°, 18.1±0.2°, 19.9±0.2°, and 22.4±0.2° as described in US 2020-0123168, incorporated herein by reference in its entirety, and Examples 3-5 below. In some embodiments, Compound I, or its pharmaceutically acceptable salt, Compound IA, or Compound IA, Form B, is administered as an oral, solid dosage form of between about 100 mg to 650 mg, or alternatively about 100 mg, 150 mg, 200 mg, 300 mg, 400 mg, 500 mg, or 650 mg dosed once a day. In some embodiments, the Compound I, or its pharmaceutically acceptable salt thereof, Compound IA, or Compound IA, Form B is administered as an oral dose of between 100 mg and 250 mg, or alternatively about 100 mg, 150 mg, 200 mg, or 250 mg twice a day, optionally spaced about 12 hours apart. In a particular embodiment, Compound I, or its pharmaceutically acceptable salt thereof, Compound IA, or Compound IA, Form B is administered as an oral dose of about 150 mg twice daily. In some embodiments, Compound I, or its pharmaceutically acceptable salt thereof, Compound IA, or Compound IA, Form B is administered as an oral dose of about 150 mg twice daily in a solid dosage form including, but not limited to, a solid tablet or capsule.

In a further alternative embodiment, the CDK4/6 inhibitor having the structure

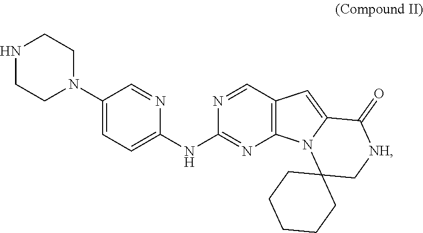 [SMILES: O=C1NCC2(CCCCC2)n2c1cc1cnc(Nc3ccc(N4CCNCC4)cn3)nc12]

or its pharmaceutically acceptable salt, is administered as the composition for use as described herein. Compound II can be administered orally or intravenously. Compound II can be prepared as previously described in US 2014-0271466, incorporated herein by reference.

Compound III, known as Trilaciclib (2′-((5-(4-methylpiperazin-1-yl)pyridin-2-yl)amino)-7′,8′-dihydro-6′H-spiro(cyclohexane-1,9′-pyrazino(1′,2′:1,5)pyrrolo(2,3-d)pyrimidin)-6′-one) is a highly selective CDK4/6 inhibitor having the structure:

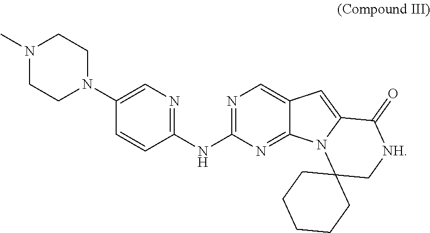 [SMILES: CN1CCN(c2ccc(Nc3ncc4cc5n(c4n3)C3(CCCCC3)CNC5=O)nc2)CC1]

As provided herein, trilaciclib or its pharmaceutically acceptable salt, composition, isotopic analog, or prodrug thereof is the composition for use as described herein. Trilaciclib can be administered in a suitable carrier. Trilaciclib is described in US 2013-0237544, incorporated herein by reference in its entirety. Trilaciclib can be synthesized as described in or US 2019-0135820, incorporated herein by reference in its entirety. Trilaciclib can be administered in any manner that achieves the desired outcome, including systemically, parenterally, intravenously, intramuscularly, subcutaneously, or intradermally. For injection, trilaciclib may be provided, in some embodiments, for example, as a 300 mg/vial as a sterile, lyophilized, yellow cake providing 300 mg of trilaciclib (equivalent to 349 mg of trilaciclib dihydrochloride). The product, for example, may be supplied in single-use 20-mL clear glass vials and does not contain a preservative. Prior to administration, trilaciclib for injection, 300 mg/vial may be reconstituted with 19.5 ml of 0.9% sodium chloride injection or 5% dextrose injection. This reconstituted solution has a trilaciclib concentration of 15 mg/mL and would typically be subsequently diluted prior to intravenous or other route administration. In some embodiments, Compound III is administered once a day parenterally, intravenously, intramuscularly, subcutaneously, or intradermally. In some embodiments, Compound III is administered once every other day, once every third day, once a week, once every 10 days, once every 14 days, once every 21 days, or once every 28 days parenterally, intravenously, intramuscularly, subcutaneously, or intradermally. In some embodiments, Compound III is administered at a dose of between about 180 mg/m2 and 300 mg/m². In some embodiment, Compound III is administered systemically, parenterally, intravenously, intramuscularly, subcutaneously, or intradermally at about 240 mg/m².

In a further alternative embodiment, the CDK4/6 inhibitor having the structure

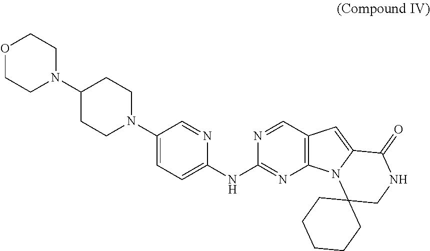 [SMILES: O=C1NCC2(CCCCC2)n2c1cc1cnc(Nc3ccc(N4CCC(N5CCOCC5)CC4)cn3)nc12]

or its pharmaceutically acceptable salt, is administered as the composition for use as described herein. Compound IV can be administered orally or intravenously. Compound IV can be prepared as previously described in US 2014-0271466, incorporated herein by reference.

In a further alternative embodiment, the CDK4/6 inhibitor having the structure

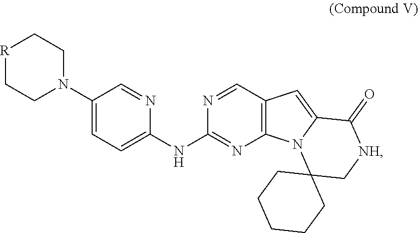

wherein R is C(H)X, NX, C(H)Y, or C(X)₂,

where X is hydrogen or straight, branched or cyclic C₁ to C₅ alkyl group, including methyl, ethyl, propyl, cyclopropyl, isopropyl, butyl, sec-butyl, tert-butyl, isobutyl, cyclobutyl, pentyl, isopentyl, neopentyl, tert-pentyl, sec-pentyl, and cyclopentyl; and

Y is NR₁R₂ wherein R₁ and R₂ are independently X, or wherein R₁ and R₂ are alkyl groups that together form a bridge that includes one or two heteroatoms (N, O, or S);

and wherein two X groups can together form an alkyl bridge or a bridge that includes one or two heteroatoms (N, S, or O) to form a spiro compound, or its pharmaceutically acceptable salt, is the composition for use as described herein. Compound V can be administered orally or intravenously. Compound V can be prepared as previously described in US 2014-0271466, incorporated herein by reference.

In a further alternative embodiment, the CDK4/6 inhibitor having the structure

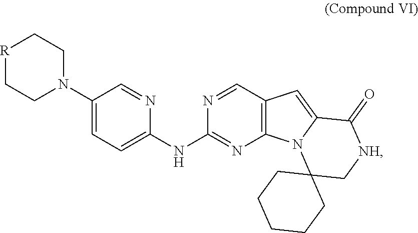

wherein R is NX, and wherein X is hydrogen, methyl, or isopropyl is the composition for use as described herein.

In an alternative embodiment, a CDK4/6 inhibitor other than those specifically described above can be used in the present invention. Non-limiting examples include palbociclib, abemaciclib, and ribociclib.

FGFR Inhibitors

The present invention provides compositions and treatments for treating a host with a cancer with dysregulated FGFR signaling, wherein the treatment comprises administering to the host a selective CDK4/6 inhibitor described herein in combination or alternation with an FGFR inhibitor as described herein. FGFR inhibitors for use in the present invention can be selected from nonselective FGFR inhibitors, selective FGFR inhibitors, FGFR monoclonal antibodies, and FGF traps. In particular embodiments, the FGFR inhibitors for use herein are selective FGFR inhibitors.

Selective FGFR inhibitors for use as a composition for the treatments described herein include, but are not limited to, erdafitinib, infigratinib, pemigatinib, AZD4547, futibatinib (TAS-120), derazantinib, roblitinib, LY287445, INCB062079, BLU9931, PRN1371, FIIN-2, PD173074, H3B-6527, fisogatinib, alofanib, bemarituzumab, vofatamab, MGFR1877S, and Debio1347, or a pharmaceutically acceptable salt of any thereof. In some embodiments, the FGFR inhibitor is not erdafitinib

In alternative embodiments, a nonselective FGFR inhibitor may be used as a composition for use in the treatments described herein including, but are not limited to, dovitinib (Oncology Venture A/S), lucitanib (Clovis Oncology), lenvatinib (Eisai Pharmaceuticals, LENVIMA™), regorafenib (Bayer, STIVARGA™), ponatinib (Ariad Pharmaceuticals, INCLUSIG™), nintedanib (Boehringer Ingelheim, OFEV™), SOMCL-085, pazopanib (Novartis, VOTRIENT™) or orantinib (Taiho Pharmaceuticals), or a pharmaceutically acceptable salt of any thereof.

In alternative embodiments, FGFR monoclonal antibodies for use in the treatments described herein include, but are not limited to, bemarituzumab (FPA144, Five Prime Therapeutics), MGFR1877S (Genentech) and vofatamab (B-701, Rainier Therapeutics). In some embodiments, a CDK4/6 inhibitor selected from Compounds I-V is administered in combination or alternation with bemarituzumab. In some embodiments, a CDK4/6 inhibitor selected from Compounds I-V is administered in combination or alternation with MGFR1877S. In some embodiments, a CDK4/6 inhibitor selected from Compounds I-VI is administered in combination or alternation with vofatamab. In some embodiments, the CDK4/6 inhibitor is Compound I, Compound IA, or Compound IA, Form B. In alternative embodiments, the CDK4/6 inhibitor is Compound III.

In an alternative embodiment, FGF ligand traps are the compositions for use in the treatments described herein. In some embodiments, a CDK4/6 inhibitor selected from Compounds I-VI is administered in combination or alternation with GSK3052230. In particular embodiments, the CDK4/6 inhibitor is Compound I, Compound IA, or Compound IA, Form B. In alternative embodiments, the CDK4/6 inhibitor is Compound III.

In some embodiments, the FGFR inhibitors for use in the present invention include, but are not limited to, the FGFR inhibitors described below, or their pharmaceutically acceptable salts thereof:

Erdafitinib (Janssen, BALVERSA™) is a selective kinase inhibitor that binds to and inhibits enzymatic activity of FGFR1, FGFR2, FGFR3 and FGFR4. Erdafitinib has the chemical structure:

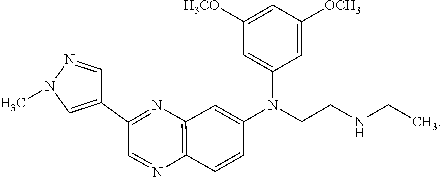 [SMILES: CCNCCN(c1cc(OC)cc(OC)c1)c1ccc2ncc(-c3cnn(C)c3)nc2c1]

Erdafitinib has been approved for treatment of metastatic urothelial cancer with an FGFR3 or FGFR2 alteration that has progressed beyond traditional platinum-based therapies. Erdafitinib is administered at an initial dose of 8 mg once daily based on serum phosphate levels and tolerability at 14 to 21 days. The dose is generally increased to 9 mg once daily if serum phosphate levels is <5.5 mg/dl and no ocular disorders or grade 2 or greater adverse reactions have occurred. Accordingly, in some embodiments, a CDK4/6 inhibitor described herein is administered daily in combination with erdafitinib to treat a cancer with FGFR dysregulated signaling or aberration, wherein the CDK4/6 inhibitor and erdafitinib are administered for at least 21 days, 24 days, 28 days, 35 days, 42 days, 56 days, or more. In some embodiments, the CDK4/6 inhibitor is Compound I, Compound IA, or Compound IA, Form B is administered at about 100 mg-200 mg twice a day, for example 150 mg, and erdafitanib is administered at about 8 mg or 9 mg once a day. In alternative embodiments, the CDK4/6 inhibitor is Compound III. In some embodiment, Compound III is administered systemically, parenterally, intravenously, intramuscularly, subcutaneously, or intradermally at about 240 mg/m².

Dovitinib (Oncology Venture A/S) strongly binds to fibroblast growth factor receptor 3 (FGFR3) and inhibits its phosphorylation, which results in the inhibition of tumor cell proliferation and the induction of tumor cell death. Dovitinib has the chemical structure:

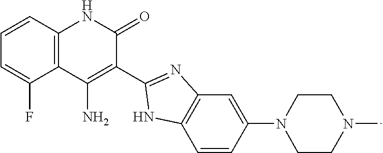

Lucitanib (Clovis Oncology) is a protein kinase inhibitor that blocks the VEGF receptors 1, 2 and 3, as well as the fibroblast growth factor receptors 1 and 2, and the platelet-derived growth factor receptors alpha and beta. Lucitinib has the chemical structure:

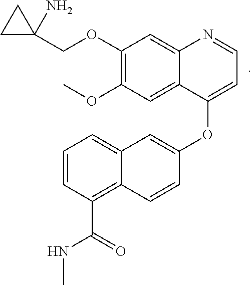 [SMILES: CNC(=O)c1cccc2cc(Oc3ccnc4cc(OCC5(N)CC5)c(OC)cc34)ccc12]

Accordingly, in some embodiments, a CDK4/6 inhibitor described herein is administered daily in combination with lucitinib to treat a cancer with FGFR dysregulated signaling or aberration, wherein the CDK4/6 inhibitor and lucitinib are administered for at least 21 days, 24 days, 28 days, 35 days, 42 days, 56 days, or more. In some embodiments, the CDK4/6 inhibitor is Compound I, Compound IA, or Compound IA, Form B is administered at about 100 mg-200 mg twice a day, for example 150 mg, and lucitinib is administered at between about 5 mg to 10 mg once a day. In alternative embodiments, the CDK4/6 inhibitor is Compound III. In some embodiment, Compound III is administered systemically, parenterally, intravenously, intramuscularly, subcutaneously, or intradermally at about 240 mg/m².

Lenvatinib (Eisai Pharmaceuticals, LENVIMA™) has been approved for the treatment of differentiated thyroid cancer that is either locally recurrent or metastatic, progressive, and did not respond to treatment with radioactive iodine (radioiodine), and in combination with everolimus for the treatment of advanced renal cell carcinoma following one prior anti-angiogenic therapy. Lenvatinib has the chemical structure:

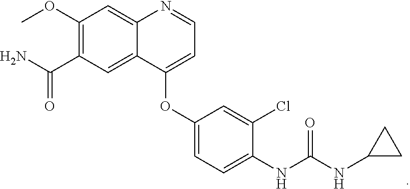 [SMILES: COc1cc2nccc(Oc3ccc(NC(=O)NC4CC4)c(Cl)c3)c2cc1C(N)=O]

Lenvatinib is a kinase inhibitor that inhibits the kinase activities of vascular endothelial growth factor (VEGF) receptors VEGFR1 (FLT1), VEGFR2 (KDR), and VEGFR3 (FLT4). Lenvatinib inhibits other kinases that have been implicated in pathogenic angiogenesis, tumor growth, and cancer progression in addition to their normal cellular functions, including fibroblast growth factor (FGF) receptors FGFR1, 2, 3, and 4; platelet derived growth factor receptor alpha (PDGFRα), and KIT. Lenvatinib also exhibited antiproliferative activity in hepatocellular carcinoma cell lines dependent on activated FGFR signaling with a concurrent inhibition of FGF-receptor substrate 2α (FRS2α) phosphorylation. Accordingly, in some embodiments, a CDK4/6 inhibitor described herein is administered daily in combination with lenvatinib to treat a cancer with FGFR dysregulated signaling or aberration, wherein the CDK4/6 inhibitor and erdafitinib are administered for at least 21 days, 24 days, 28 days, 35 days, 42 days, 56 days, or more. In some embodiments, the CDK4/6 inhibitor is Compound I, Compound IA, or Compound IA, Form B is administered at about 100 mg-200 mg twice a day, for example 150 mg, and lenvatinib is administered at between about 8 mg to 24 mg once a day. In alternative embodiments, the CDK4/6 inhibitor is Compound III. In some embodiment, Compound III is administered systemically, parenterally, intravenously, intramuscularly, subcutaneously, or intradermally at about 240 mg/m².

Regorafenib (Bayer, STIVARGA™) is an oral multi-kinase inhibitor developed by Bayer which targets angiogenic, stromal and oncogenic receptor tyrosine kinase (RTK). Regorafenib has the chemical structure:

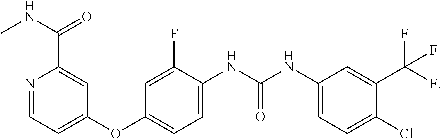 [SMILES: CNC(=O)c1cc(Oc2ccc(NC(=O)Nc3ccc(Cl)c(C(F)(F)F)c3)c(F)c2)ccn1]

Regorafenib has been approved for the treatment of colorectal cancer, gastrointestinal stromal tumors, and hepatocellular carcinoma and is administered at 160 mg orally, once daily for the first 21 days of each 28-day cycle. In some embodiments, provided herein are compositions and treatments for treating a cancer with an FGFR dysregulated signaling or aberration, wherein the treatment comprises administering a CDK4/6 inhibitor described herein and regorafenib, wherein the CDK4/6 inhibitor and regorafenib are administered daily for 21-days of a 28-day cycle. In some embodiments, a CDK4/6 inhibitor described herein is administered daily for 28 days of a 28-day cycle, and regorafenib is administered daily for 21 days of a 28-day cycle. In some embodiments, the CDK4/6 inhibitor is Compound I, Compound IA, or Compound IA, Form B is administered at about 100 mg-200 mg twice a day, for example 150 mg, and regorafenib is administered at between about 150 mg and 180 mg once a day, wherein the CDK4/6 inhibitor is administered daily for at least 21 days, at least 24 days, or 28 days of a 28-day cycle and regorafenib is administered daily for 21 days of a 28-day cycle. In alternative embodiments, the CDK4/6 inhibitor is Compound III. In some embodiment, Compound III is administered systemically, parenterally, intravenously, intramuscularly, subcutaneously, or intradermally at about 240 mg/m².

Ponatinib (Ariad Pharmaceuticals, INCLUSIG™) is an oral drug developed by ARIAD Pharmaceuticals for the treatment of chronic myeloid leukemia (CML) and Philadelphia chromosome-positive (Ph+) acute lymphoblastic leukemia (ALL). It is a multi-targeted tyrosine-kinase inhibitor having the chemical structure:

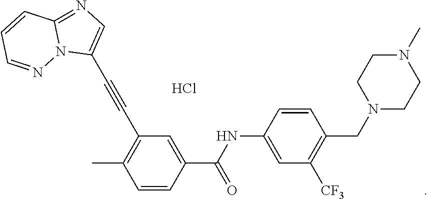 [SMILES: Cc1ccc(C(=O)Nc2ccc(CN3CCN(C)CC3)c(C(F)(F)F)c2)cc1C#Cc1cnc2cccnn12 ; Cl]

Accordingly, in some embodiments, a CDK4/6 inhibitor described herein is administered daily in combination with ponatinib to treat a cancer with FGFR dysregulated signaling or aberration, wherein the CDK4/6 inhibitor and ponatinib are administered for at least 21 days, 24 days, 28 days, 35 days, 42 days, 56 days, or more. In some embodiments, the CDK4/6 inhibitor is Compound I, Compound IA, or Compound IA, Form B is administered at about 100 mg-200 mg twice a day, for example 150 mg, and ponatinib is administered at between about 30 mg to 45 mg once a day. In alternative embodiments, the CDK4/6 inhibitor is Compound III. In some embodiment, Compound III is administered systemically, parenterally, intravenously, intramuscularly, subcutaneously, or intradermally at about 240 mg/m².

Nintedanib (Boehringer Ingelheim, OFEV™) marketed under the brand names Ofev and Vargatef, is an oral medication used for the treatment of idiopathic pulmonary fibrosis and along with other medications for some types of non-small-cell lung cancer. Nintedanib has the chemical structure:

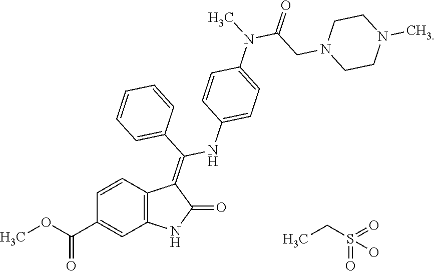

Accordingly, in some embodiments, a CDK4/6 inhibitor described herein is administered daily in combination with nintedanib to treat a cancer with FGFR dysregulated signaling or aberration, wherein the CDK4/6 inhibitor and nintedanib are administered for at least 21 days, 24 days, 28 days, 35 days, 42 days, 56 days, or more. In some embodiments, the CDK4/6 inhibitor is Compound I, Compound IA, or Compound IA, Form B is administered at about 100 mg-200 mg twice a day, for example 150 mg, and nintedanib is administered at between about 100 mg to about 200 mg twice daily, for example 150 mg twice daily approximately 12 hours apart. In alternative embodiments, the CDK4/6 inhibitor is Compound III. In some embodiment, Compound III is administered systemically, parenterally, intravenously, intramuscularly, subcutaneously, or intradermally at about 240 mg/m².

Pazopanib (Novartis, VOTRIENT™) is a potent and selective multi-targeted receptor tyrosine kinase inhibitor that blocks tumour growth and inhibits angiogenesis. It has been approved for renal cell carcinoma and soft tissue sarcoma. Pazopanib has the chemical structure:

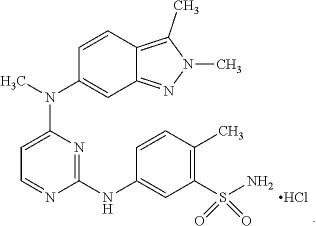 [SMILES: Cc1ccc(Nc2nccc(N(C)c3ccc4c(C)n(C)nc4c3)n2)cc1S(N)(=O)=O ; Cl]

Accordingly, in some embodiments, a CDK4/6 inhibitor described herein is administered daily in combination with pazopanib to treat a cancer with FGFR dysregulated signaling or aberration, wherein the CDK4/6 inhibitor and pazopanib are administered for at least 21 days, 24 days, 28 days, 35 days, 42 days, 56 days, or more. In some embodiments, the CDK4/6 inhibitor is Compound I, Compound IA, or Compound IA, Form B is administered at about 100 mg-200 mg twice a day, for example 150 mg, and pazopanib is administered at between about 200 mg to about 800 mg once daily. In alternative embodiments, the CDK4/6 inhibitor is Compound III. In some embodiment, Compound III is administered systemically, parenterally, intravenously, intramuscularly, subcutaneously, or intradermally at about 240 mg/m².

Orantinib (Taiho Pharmaceuticals) is an orally bioavailable receptor tyrosine kinase inhibitor. Orantinib binds to and inhibits the autophosphorylation of vascular endothelial growth factor receptor 2 (VEGFR2), platelet-derived growth factor receptor (PDGFR), and fibroblast growth factor receptor (FGFR), thereby inhibiting angiogenesis and cell proliferation. Orantinib also inhibits the phosphorylation of the stem cell factor receptor tyrosine kinase c-kit, often expressed in acute myelogenous leukemia cells. Orantinib has the chemical structure:

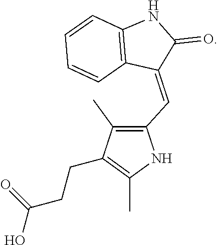 [SMILES: Cc1[nH]c(/C=C2/C(=O)Nc3ccccc32)c(C)c1CCC(=O)O]

Accordingly, in some embodiments, a CDK4/6 inhibitor described herein is administered daily in combination with orantinib to treat a cancer with FGFR dysregulated signaling or aberration, wherein the CDK4/6 inhibitor and orantinib are administered for at least 21 days, 24 days, 28 days, 35 days, 42 days, 56 days, or more. In some embodiments, the CDK4/6 inhibitor is Compound I, Compound IA, or Compound IA, Form B is administered at about 100 mg-200 mg twice a day, for example 150 mg, and orantinib is administered at between about 150 mg and 250 mg twice per day, for example 200 mg twice per day. In alternative embodiments, the CDK4/6 inhibitor is Compound III. In some embodiment, Compound III is administered systemically, parenterally, intravenously, intramuscularly, subcutaneously, or intradermally at about 240 mg/m².

Infigratinib (BGJ398, QED Therapeutics) is an orally bioavailable pan inhibitor of human fibroblast growth factor receptors (FGFRs) with potential antiangiogenic and antineoplastic activities. Infigratinib selectively binds to and inhibits the activities of FGFRs, resulting in the inhibition of tumor angiogenesis and tumor cell proliferation, and the induction of tumor cell death. Infigratinib has the chemical structure:

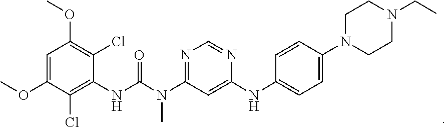 [SMILES: CCN1CCN(c2ccc(Nc3cc(N(C)C(=O)Nc4c(Cl)c(OC)cc(OC)c4Cl)ncn3)cc2)CC1]

Infigratinib is administered once daily for 21-days during a 28-day cycle. In clinical trials, infigratinib has been administered at a dose of about between 50 and 150 mg once daily. In some embodiments, provided herein is a method of treating a cancer with an FGFR dysregulated signaling or aberration by administering a CDK4/6 inhibitor described herein and infigratinib, wherein the CDK4/6 inhibitor and infigratinib are administered daily for 21-days of a 28-day cycle. In some embodiments, a CDK4/6 inhibitor described herein is administered daily for 28 days of a 28-day cycle, and infigratinib is administered daily for 21 days of a 28-day cycle. In some embodiments, the CDK4/6 inhibitor is Compound I, Compound IA, or Compound IA, Form B is administered at about 100 mg-200 mg twice a day, for example 150 mg, and infigratinib is administered at between about 50 mg and 150 mg once a day, wherein the CDK4/6 inhibitor is administered daily for at least 21 days, at least 24 days, or 28 days of a 28-day cycle and infigratinib is administered daily for 21 days of a 28-day cycle. In alternative embodiments, the CDK4/6 inhibitor is Compound III. In some embodiment, Compound III is administered systemically, parenterally, intravenously, intramuscularly, subcutaneously, or intradermally at about 240 mg/m².

Pemigatinib (Pemazyre; INCB54828, Incyte) is a selective FGFR inhibitor approved for the treatment of patients with cholangiocarcinoma, having the chemical structure:

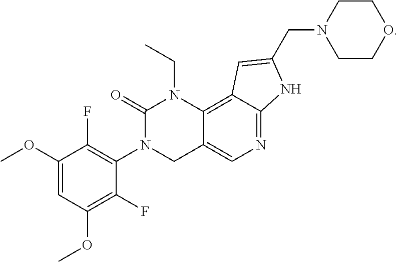 [SMILES: CCN1C(=O)N(c2c(F)c(OC)cc(OC)c2F)Cc2cnc3[nH]c(CN4CCOCC4)cc3c21]

Pemigatinib has been approved to treat previously treated, unresectable locally advanced or metastatic cholangiocarcinoma with a fibroblast growth factor receptor 2 (FGFR2) fusion or other rearrangement as detected by an FDA approved test. Pemigatinib is approved for daily administration of 13.5 mg for 14 days of a 21-day cycle. In some embodiments, provided herein is a method of treating a cancer with an FGFR dysregulated signaling or aberration by administering a CDK4/6 inhibitor described herein and pemigatinib, wherein the CDK4/6 inhibitor and pemigatinib are administered daily for 14-days of a 21-day cycle. In some embodiments, a CDK4/6 inhibitor described herein is administered daily for 21 days of a 21-day cycle, and pemigatinib is administered daily for 21 days of a 28-day cycle. In some embodiments, the CDK4/6 inhibitor is Compound I, Compound IA, or Compound IA, Form B is administered at about 100 mg-200 mg twice a day, for example 150 mg, and pemigatinib is administered at between about 10 mg and 15 mg once a day, for example about 13.5 mg, wherein the CDK4/6 inhibitor is administered daily for at least 14 days, at least 17 days, or 21 days of a 21-day cycle and infigratinib is administered daily for 14 days of a 28-day cycle. In alternative embodiments, the CDK4/6 inhibitor is Compound III. In some embodiment, Compound III is administered systemically, parenterally, intravenously, intramuscularly, subcutaneously, or intradermally at about 240 mg/m².

AZD4547 (AstraZeneca) is an orally bioavailable inhibitor of the fibroblast growth factor receptor (FGFR) with antineoplastic activity. FGFR inhibitor AZD4547 binds to and inhibits FGFR, which may result in the inhibition of FGFR-related signal transduction pathways, and, so, the inhibition of tumor cell proliferation and tumor cell death. AZD4547 has the chemical structure:

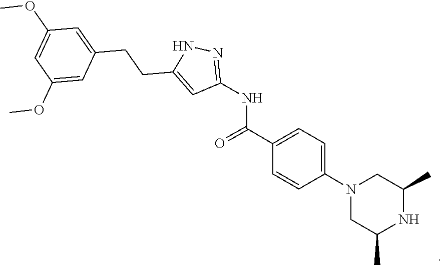

In some embodiments, provided herein is a method of treating a cancer with an FGFR dysregulated signaling or aberration by administering a CDK4/6 inhibitor described herein and AZD4547, wherein the CDK4/6 inhibitor and AZD4547 are administered daily on a continuous schedule, for example at least 21 days, 28 days, 35 days or more. In some embodiments, a CDK4/6 inhibitor described herein is administered daily for 28 days of a 28-day cycle, and AZD4547 is administered daily for 21 days of a 28-day cycle. In some embodiments, the CDK4/6 inhibitor is Compound I, Compound IA, or Compound IA, Form B is administered at about 100 mg-200 mg twice a day, for example 150 mg, and AZD4547 is administered at between about 60 mg and 100 mg twice a day, for example about 80 mg, wherein the CDK4/6 inhibitor is administered daily for at least 21 days, at least 24 days, or 28 days of a 28-day cycle and AZD4547 is administered daily for at least 21 days, at least 24 days, or 28 days of a 28-day cycle. In alternative embodiments, the CDK4/6 inhibitor is Compound III. In some embodiment, Compound III is administered systemically, parenterally, intravenously, intramuscularly, subcutaneously, or intradermally at about 240 mg/m².

Futibatinib (TAS-120; Taiho Pharmaceuticals) is highly selective orally bioavailable inhibitor of the fibroblast growth factor receptor (FGFR) with antineoplastic activity. TAS-120 has the chemical structure:

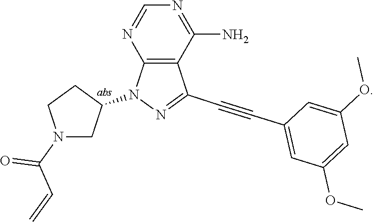 [SMILES: C=CC(=O)N1CC[C@H](n2nc(C#Cc3cc(OC)cc(OC)c3)c3c(N)ncnc32)C1]

In some embodiments, provided herein are compositions and treatments for treating a cancer with an FGFR dysregulated signaling or aberration, wherein the treatment comprises administering a CDK4/6 inhibitor described herein and futibatinib, wherein the CDK4/6 inhibitor and futibatinib are administered daily on a continuous schedule, for example at least 21 days, 28 days, 35 days or more. In some embodiments, the CDK4/6 inhibitor is Compound I, Compound IA, or Compound IA, Form B is administered at about 100 mg-200 mg twice a day, for example 150 mg, and futibatinib is administered at between about 10 mg and 30 mg once a day, for example about 20 mg. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

Derazantinib (Arqule, ARQ087) is an orally bioavailable inhibitor of the fibroblast growth factor receptor (FGFR) with IC50 values of 1.8 nM for FGFR2, and 4.5 nM for FGFR1 and 3, showing lower potency for FGFR4 (IC50=34 nM). Derazantinib has the chemical structure:

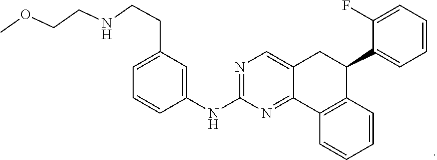 [SMILES: COCCNCCc1cccc(Nc2ncc3c(n2)-c2ccccc2[C@H](c2ccccc2F)C3)c1]

In some embodiments, provided herein are compositions and treatments for treating a cancer with an FGFR dysregulated signaling or aberration, wherein the treatment comprises administering a CDK4/6 inhibitor described herein and derazantinib, wherein the CDK4/6 inhibitor and derazantinib are administered daily on a continuous schedule, for example at least 21 days, 28 days, 35 days or more. In some embodiments, the CDK4/6 inhibitor is Compound I, Compound IA, or Compound IA, Form B is administered at about 100 mg-200 mg twice a day, for example 150 mg, and derazantinib is administered at between about 10 mg and 30 mg once a day, for example about 20 mg. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

Roblitinib (FGF-401, Novartis) is an FGFR4-selective inhibitor with an IC50 of 1.1 nM. It binds in a reversible covalent manner to the FGFR4 kinase domain and shows at least 1,000 fold selectivity against of panel of 65 kinases in biochemical assays. Roblitinib has the chemical structure:

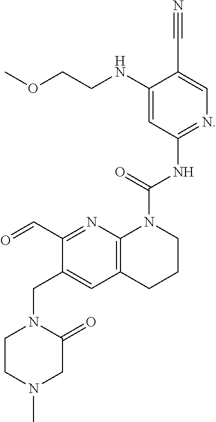 [SMILES: COCCNc1cc(NC(=O)N2CCCc3cc(CN4CCN(C)CC4=O)c(C=O)nc32)ncc1C#N]

In some embodiments, provided herein are compositions and treatments for treating a cancer with an FGFR dysregulated signaling or aberration, wherein the treatment comprises administering a CDK4/6 inhibitor described herein and roblitinib, wherein the CDK4/6 inhibitor and roblitinib are administered daily on a continuous schedule, for example at least 21 days, 28 days, 35 days or more. In some embodiments, the CDK4/6 inhibitor is Compound I, Compound IA, or Compound IA, Form B is administered at about 100 mg-200 mg twice a day, for example 150 mg twice a day. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

Debio1347 (Debiopharm), also known as CH5183284, is a selective and orally available FGFR inhibitor with IC50 of 9.3 nM, 7.6 nM, 22 nM, and 290 nM for FGFR1, FGFR2, FGFR3, and FGFR4, respectively. Debio1347 has the chemical structure:

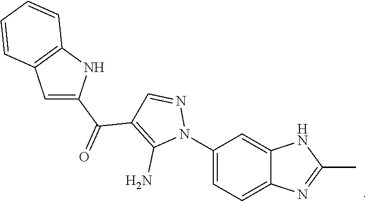 [SMILES: Cc1nc2ccc(-n3ncc(C(=O)c4cc5ccccc5[nH]4)c3N)cc2[nH]1]

In some embodiments, provided herein is a method treating a cancer with an FGFR dysregulated signaling or aberration by administering a CDK4/6 inhibitor described herein and Debio1347, wherein the CDK4/6 inhibitor and Debio1347 are administered daily on a continuous schedule, for example at least 21 days, 28 days, 35 days or more. In some embodiments, the CDK4/6 inhibitor is Compound I, Compound IA, or Compound IA, Form B is administered at about 100 mg-200 mg twice a day, for example 150 mg, and Debio1347 is administered at between about 10 mg and 210 mg once a day, for example about 80 mg twice a day. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

BLU9931 (Blueprint Medicines) is a FGFR4-selective inhibitor with an IC50 of 3.0 nM. It binds in an irreversible covalent manner to the FGFR4 kinase domain. BLU9931 binds within the ATP-binding pocket of FGFR4, forming a covalent bond with Cys552. BLU9931 has the chemical structure:

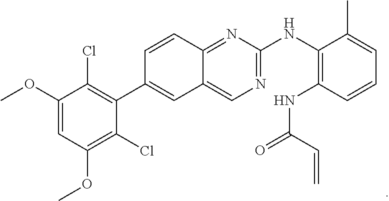 [SMILES: C=CC(=O)Nc1cccc(C)c1Nc1ncc2cc(-c3c(Cl)c(OC)cc(OC)c3Cl)ccc2n1]

In some embodiments, provided herein are compositions and treatments for treating a cancer with an FGFR dysregulated signaling or aberration, wherein the treatment comprises administering a CDK4/6 inhibitor described herein and BLU9931, wherein the CDK4/6 inhibitor and BLU9931 are administered daily on a continuous schedule, for example at least 14 days, at least 21 days, at least 28 days, at least 35 days or more. In some embodiments, a CDK4/6 inhibitor described herein is administered daily for 28 days of a 28-day cycle, and BLU9931 is administered daily for 21 days of a 28-day cycle. In some embodiments, the CDK4/6 inhibitor is Compound I, Compound IA, or Compound IA, Form B is administered at about 100 mg-200 mg twice a day, for example 150 mg, and BLU9931 is administered at between about 10 mg and 250 mg, wherein the CDK4/6 inhibitor is administered daily for at least 21 days, at least 24 days, or 28 days of a 28-day cycle and BLU9931 is administered daily for at least 21 days, at least 24 days, or 28 days of a 28-day cycle. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

PRN1371 (Principia Biopharma) is a highly selective and potent FGFR1-4 and CSF1R inhibitor with IC50s of 0.6, 1.3, 4.1, 19.3 and 8.1 nM for FGFR1, FGFR2, FGFR3, FGFR4 and CSF1R, respectively. PRN1371 targets a cysteine residue within the kinase domain. PRN1371 has the chemical structure:

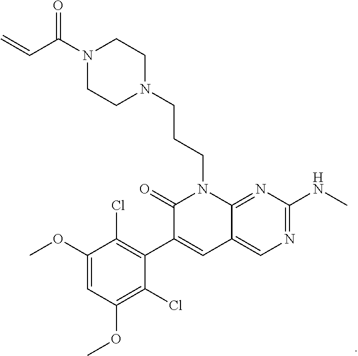 [SMILES: C=CC(=O)N1CCN(CCCn2c(=O)c(-c3c(Cl)c(OC)cc(OC)c3Cl)cc3cnc(NC)nc32)CC1]

In some embodiments, provided herein are compositions and treatments for treating a cancer with an FGFR dysregulated signaling or aberration, wherein the treatment comprises administering a CDK4/6 inhibitor described herein and PRN1371, wherein the CDK4/6 inhibitor and PRN1371 are administered daily on a continuous schedule, for example at least 14 days, at least 21 days, at least 28 days, at least 35 days or more. In some embodiments, a CDK4/6 inhibitor described herein is administered daily for 21 days of a 28-day cycle, and PRN1371 is administered daily for 28 days of a 28-day cycle. In some embodiments, the CDK4/6 inhibitor is Compound I, Compound IA, or Compound IA, Form B is administered at about 100 mg-200 mg twice a day, for example 150 mg, and PRN1371 is administered at between about 10 mg and 500 mg, wherein the CDK4/6 inhibitor is administered daily for at least 21 days, at least 24 days, or 28 days of a 28-day cycle and PRN1371 is administered daily for at least 21 days, at least 24 days, or 28 days of a 28-day cycle. In some embodiments, the cancer with FGFR dysregulated signaling is an FGF19 overexpression or amplification. In some embodiments, the cancer is FGF19-positive hepatocellular carcinoma (HCC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

PD173074 (Pfizer) is a potent FGFR1 inhibitor with IC50 of ˜25 nM and also inhibits VEGFR2 with IC50 of 100-200 nM in cell-free assays, ˜1000-fold selective for FGFR1 than PDGFR and c-Src. PD173074 has the chemical structure:

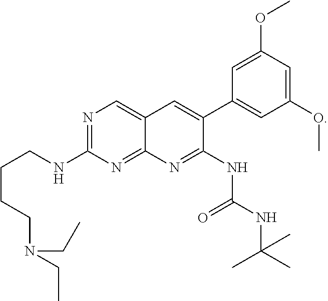 [SMILES: CCN(CC)CCCCNc1ncc2cc(-c3cc(OC)cc(OC)c3)c(NC(=O)NC(C)(C)C)nc2n1]

In some embodiments, provided herein are compositions and treatments for treating a cancer with an FGFR dysregulated signaling or aberration, wherein the treatment comprises administering a CDK4/6 inhibitor described herein and PD173074, wherein the CDK4/6 inhibitor and PD173074 are administered daily on a continuous schedule, for example at least 14 days, at least 21 days, at least 28 days, at least 35 days or more. In some embodiments, a CDK4/6 inhibitor described herein is administered daily for 21 days of a 28-day cycle, and PD173074 is administered daily for 21 days of a 28-day cycle. In some embodiments, the CDK4/6 inhibitor is Compound I, Compound IA, or Compound IA, Form B is administered at about 100 mg-200 mg twice a day, for example 150 mg, and PD173074 is administered at between about 10 mg and 500 mg, wherein the CDK4/6 inhibitor is administered daily for at least 21 days, at least 24 days, or 28 days of a 28-day cycle and PD173074 is administered daily for at least 21 days, at least 24 days, or 28 days of a 28-day cycle. In alternative embodiments, the CDK4/6 inhibitor is Compound III. In some embodiment, Compound III is administered systemically, parenterally, intravenously, intramuscularly, subcutaneously, or intradermally at about 240 mg/m².

FIIN-2 is an irreversible, pan-FGFR inhibitor with IC50 of 3.09 nM, 4.3 nM, 27 nM and 45.3 nM for FGFR1/2/3/4, respectively. FIIN-2 has the chemical structure:

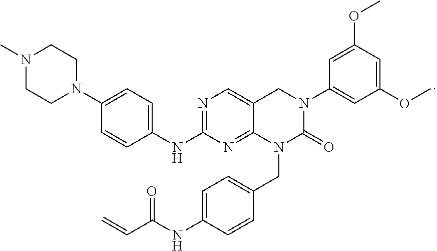

In some embodiments, provided herein are compositions and treatments for treating a cancer with an FGFR dysregulated signaling or aberration, wherein the treatment comprises administering a CDK4/6 inhibitor described herein and FIIN-2, wherein the CDK4/6 inhibitor and PD173074 are administered daily on a continuous schedule, for example at least 14 days, at least 21 days, at least 28 days, at least 35 days or more. In some embodiments, a CDK4/6 inhibitor described herein is administered daily for 21 days of a 28-day cycle, and FIIN-2 is administered daily for 21 days of a 28-day cycle. In some embodiments, the CDK4/6 inhibitor is Compound I, Compound IA, or Compound IA, Form B is administered at about 100 mg-200 mg twice a day, for example 150 mg, and FIIN-2 is administered at between about 10 mg and 500 mg, wherein the CDK4/6 inhibitor is administered daily for at least 21 days, at least 24 days, or 28 days of a 28-day cycle and PD173074 is administered daily for at least 21 days, at least 24 days, or 28 days of a 28-day cycle. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

LY-2874455 is an inhibitor of FGFR that exhibits anticancer chemotherapeutic activity. LY287445 inhibits cell proliferation and tumor growth across in vitro and in vivo models of several cancers, including lung cancer, gastric cancer, and multiple myeloma. LY-2874455 has the chemical structure:

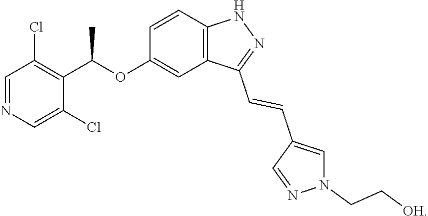 [SMILES: C[C@@H](Oc1ccc2[nH]nc(/C=C/c3cnn(CCO)c3)c2c1)c1c(Cl)cncc1Cl]

In some embodiments, provided herein are compositions and treatments for treating a cancer with an FGFR dysregulated signaling or aberration, wherein the treatment comprises administering a CDK4/6 inhibitor described herein and LY-2874455, wherein the CDK4/6 inhibitor and LY-2874455 are administered daily on a continuous schedule, for example at least 21 days, 28 days, 35 days or more. In some embodiments, the CDK4/6 inhibitor is Compound I, Compound IA, or Compound IA, Form B is administered at about 100 mg-200 mg twice a day, for example 150 mg, and LY-2874455 is administered at between about 10 mg and 30 mg twice a day, for example about 18 mg twice a day. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

Fisogatinib (BLU-554; Blueprint Medicines) is a highly selective and orally active fibroblast growth factor receptor 4 (FGFR4) inhibitor with an IC50 of 5 nM. Fisogatinib has significant anti-tumor activity in models of hepatocellular carcinoma (HCC) that are dependent on FGFR4 signaling. Fisogatinib has the chemical structure:

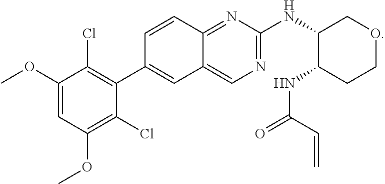 [SMILES: C=CC(=O)N[C@H]1CCOC[C@H]1Nc1ncc2cc(-c3c(Cl)c(OC)cc(OC)c3Cl)ccc2n1]

In some embodiments, provided herein are compositions and treatments for treating a cancer with an FGFR dysregulated signaling or aberration, wherein the treatment comprises administering a CDK4/6 inhibitor described herein and fisogatinib, wherein the CDK4/6 inhibitor and fisogatinib are administered daily on a continuous schedule, for example at least 21 days, 28 days, 35 days or more. In some embodiments, the CDK4/6 inhibitor is Compound I, Compound IA, or Compound IA, Form B is administered at about 100 mg-200 mg twice a day, for example 150 mg twice a day, and fisogatinib is administered at between about 300 mg and 600 mg once a day. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

H3B-6527 (H3 Biomedicine) is an inhibitor of FGF receptor 4 (FGFR4; IC50=<1.2 nM). It is selective for FGFR4 over FGFR1, FGFR2, and FGFR3 (IC50s=320, 1,290, and 1,060 nM, respectively). H3B-6527 has the chemical structure:

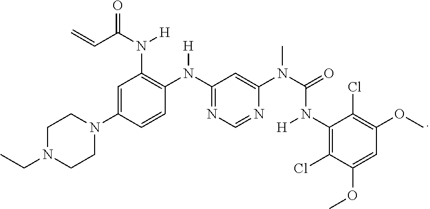 [SMILES: C=CC(=O)Nc1cc(N2CCN(CC)CC2)ccc1Nc1cc(N(C)C(=O)Nc2c(Cl)c(OC)cc(OC)c2Cl)ncn1]

In some embodiments, provided herein are compositions and treatments for treating a cancer with an FGFR dysregulated signaling or aberration, wherein the treatment comprises administering a CDK4/6 inhibitor described herein and H3B-6527, wherein the CDK4/6 inhibitor and H3B-6527 are administered daily on a continuous schedule, for example at least 21 days, 28 days, 35 days or more. In some embodiments, the CDK4/6 inhibitor is Compound I, Compound IA, or Compound IA, Form B is administered at about 100 mg-200 mg twice a day, for example 150 mg twice a day, and H3B-6527 is administered at between about 300 mg and 1200 mg, for example 1000 mg, once a day or between about 150 mg and 600 mg, for example about 500 mg, twice a day. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

SOMCL-085 is a triple inhibitor of FGFR, VEGFR, and PDGFR. SOMCL-085 potently inhibits FGFR1-3 kinase activity, with IC₅₀ values of 1.8, 1.9 and 6.9 nmol/L, respectively and displays weaker activity against FGFR4 (IC₅₀=319.9 nmol/L). SOMCL-085 has the chemical structure:

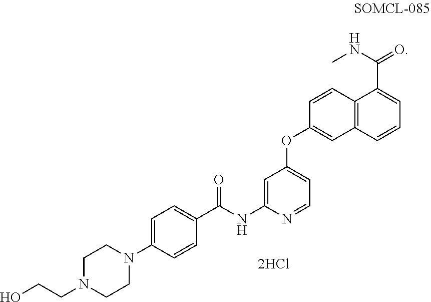 [SMILES: CNC(=O)c1cccc2cc(Oc3ccnc(NC(=O)c4ccc(N5CCN(CCO)CC5)cc4)c3)ccc12]

INCB062079 is a selective FGFR4 inhibitor that prevents tumor cell proliferation in cells with amplification and overexpression of FGF19 (see, e.g., AACR; Cancer Res 2017; 77(13 Suppl):Abstract nr 1234, PMID: 32154250). In some embodiments, provided herein is a method treating a cancer with an FGFR dysregulated signaling or aberration by administering a CDK4/6 inhibitor described herein and INCB062079, wherein the CDK4/6 inhibitor and INCB062079 are administered daily on a continuous schedule, for example at least 21 days, 28 days, 35 days or more. In some embodiments, the CDK4/6 inhibitor is Compound I, Compound IA, or Compound IA, Form B is administered at about 100 mg-200 mg twice a day, for example 150 mg twice a day, and INCB062079 is administered at between about 50 mg and 1200 mg at least once a day. In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer with FGFR dysregulated signaling is an FGF19 overexpression or amplification. In some embodiments, the cancer is FGF19-positive liver cancer, for example hepatocellular carcinoma (HCC) or intrahepatic cholangiocarcinoma (ICC). In some embodiments, the cancer is a FGF19-positive esophageal, nasopharyngeal, or ovarian cancer. In alternative embodiments, the CDK4/6 inhibitor is Compound III. In some embodiment, Compound III is administered systemically, parenterally, intravenously, intramuscularly, subcutaneously, or intradermally at about 240 mg/m².

Alofanib (RPT835) is a potent and selective allosteric inhibitor of fibroblast growth factor receptor 2 (FGFR2), having the structure:

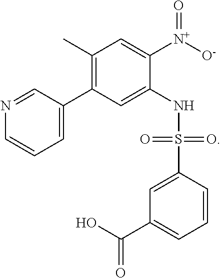 [SMILES: Cc1cc([N+](=O)[O-])c(NS(=O)(=O)c2cccc(C(=O)O)c2)cc1-c1cccnc1]

In some embodiments, provided herein are compositions and treatments for treating a cancer with an FGFR dysregulated signaling or aberration, wherein the treatment comprises administering a CDK4/6 inhibitor described herein and alofanib, wherein the CDK4/6 inhibitor and alofanib are administered daily, for example days 1 to 5 of a 7-day cycle. In some embodiments, alofanib is administered on days 1 to 5 of a 7-day cycle, and the CDK4/6 inhibitor is administered at least once a day for 7 days of a 7-day cycle. In some embodiments, the CDK4/6 inhibitor is Compound I, Compound IA, or Compound IA, Form B is administered at about 100 mg-200 mg twice a day, for example 150 mg twice a day, and alofanib is administered at between about 50 and 350 mg/m², for example 50 mg/m², 100 mg/m², 165 mg/m², 250 mg/m², or 350 mg/m² once a day. In alternative embodiments, the CDK4/6 inhibitor is Compound III. In some embodiment, Compound III is administered systemically, parenterally, intravenously, intramuscularly, subcutaneously, or intradermally at about 240 mg/m².

Bemarituzumab (FPA144, Five Prime Therapeutics) is a FGFR2b antibody in clinical development as a targeted immune therapy for tumors that over-express FGFR2b. In some embodiments, provided herein is a method treating a cancer with an FGFR dysregulated signaling or aberration by administering a CDK4/6 inhibitor described herein and bemarituzumab. In some embodiments, the CDK4/6 inhibitor is administered at least once a day for 14-days of a 14-day cycle, and the bemarituzumab is administered once every 14 days of a 14-day cycle. In some embodiments, the CDK4/6 inhibitor is Compound I, Compound IA, or Compound IA, Form B is administered at about 100 mg-200 mg twice a day, for example 150 mg twice a day, and bemarituzumab is administered at between about 0.3 mg/kg to about 15 mg/kg, for example between about 3-10 mg once every two weeks. In alternative embodiments, the CDK4/6 inhibitor is Compound III. In some embodiment, Compound III is administered systemically, parenterally, intravenously, intramuscularly, subcutaneously, or intradermally at about 240 mg/m².

MGFR1877S (Genentech) is a monoclonal antibody selective for FGFR3. In some embodiments, provided herein are compositions and treatments for treating a cancer with an FGFR dysregulated signaling or aberration, wherein the treatment comprises administering a CDK4/6 inhibitor described herein and MGFR1877S. In some embodiments, the CDK4/6 inhibitor is administered at least once a day for 14-days of a 14-day cycle, and the MGFR1877S is administered once every 14 days of a 14-day cycle. In some embodiments, the CDK4/6 inhibitor is administered at least once a day for 28-days of a 28-day cycle, and the MGFR1877S is administered once every 28 days of a 28-day cycle. In some embodiments, the CDK4/6 inhibitor is Compound I, Compound IA, or Compound IA, Form B is administered at about 100 mg-200 mg twice a day, for example 150 mg twice a day, and MGFR1877S is administered at between about 0.3 mg/kg to about 25 mg/kg, for example between about 3-10 mg once every two weeks. In alternative embodiments, the CDK4/6 inhibitor is Compound III. In some embodiment, Compound III is administered systemically, parenterally, intravenously, intramuscularly, subcutaneously, or intradermally at about 240 mg/m².

Vofatamab (B-701, Rainier Therapeutics) is an antibody that targets fibroblast growth factor receptor 3 (FGFR3). In some embodiments, provided herein are compositions and treatments for treating a cancer with an FGFR dysregulated signaling or aberration, wherein the treatment comprises administering a CDK4/6 inhibitor described herein and vofatamab. In some embodiments, the CDK4/6 inhibitor is administered at least once a day for 21-days of a 21-day cycle, and the vofatamab is administered once every 21 days of a 21-day cycle. In some embodiments, the CDK4/6 inhibitor is Compound I, Compound IA, or Compound IA, Form B is administered at about 100 mg-200 mg twice a day, for example 150 mg twice a day, and vofatamab is administered at between about 20 mg/kg to about 30 mg/kg, for example about 25 mg once every three weeks. In alternative embodiments, the CDK4/6 inhibitor is Compound III. In some embodiment, Compound III is administered systemically, parenterally, intravenously, intramuscularly, subcutaneously, or intradermally at about 240 mg/m².

GSK3052230 is a soluble fusion protein consisting of the extracellular domain of human fibroblast growth factor receptor 1 (FGFR1) fused to the Fc portion of human immunoglobulin G1 (IgG1) with antineoplastic and anti-angiogenic activities. In some embodiments, provided herein are compositions and treatments for treating a cancer with an FGFR dysregulated signaling or aberration, wherein the treatment comprises administering a CDK4/6 inhibitor described herein and GSK3052230. In some embodiments, the CDK4/6 inhibitor is administered at least once a day for 21-days of a 21-day cycle, and the GSK3052230 is administered once a week of a 21-day cycle. In some embodiments, the CDK4/6 inhibitor is Compound I, Compound IA, or Compound IA, Form B is administered at about 100 mg-200 mg twice a day, for example 150 mg twice a day, and GSK3052230 is administered at between about 5 mg/kg to about 20 mg/kg, for example about 5, 10, 15, or 20 mg/kg. In alternative embodiments, the CDK4/6 inhibitor is Compound III. In some embodiment, Compound III is administered systemically, parenterally, intravenously, intramuscularly, subcutaneously, or intradermally at about 240 mg/m².

Cancers with Dysregulation of FGFR Signaling Pathway

Fibroblast growth factor receptors (FGFR) are a subfamily of receptor tyrosine kinases which are bound by fibroblast growth factors (FGFs), which exert their pleiotropic effects by binding and activating the FGFR. The FGFR family is coded by four genes (FGFR1, FGFR2, FGFR3, and FGFR4) (see Johnson D E, Williams L T 1993. Structural and functional diversity in the FGF receptor multigene family. Adv Cancer Res 60: 1-41; Mohammadi et al., 2005b. Structural basis for fibroblast growth factor receptor activation. Cytokine Growth Factor Rev 16: 107-137). The extracellular domain of FGFRs consists of three immunoglobulin (Ig)-like domains (D1, D2, and D3), and the intracellular domain harbors the conserved tyrosine kinase domain flanked by the flexible amino-terminal juxtamembrane linker and carboxy-terminal tail (see Givol et al., 1992. Complexity of FGF receptors: Genetic basis for structural diversity and functional specificity. FASEB J 6: 3362-3369). A unique feature of FGFRs is the presence of a contiguous segment of glutamic and aspartic acids in the D1-D2 linker, termed the acid box (AB). The two-membrane proximal D2 and D3 and the intervening D2-D3 linker are necessary and sufficient for ligand binding/specificity, whereas D1 and the D1-D2 linker are implicated in receptor auto-inhibition (Kalinina et al., 2012. The alternatively spliced acid box region plays a key role in FGF receptor autoinhibition. Structure 20: 77-88).

Despite being encoded by separate genes, the four members share high homology, with their sequence identity varying from 56% to 71% (Itoh N., Ornitz D. M. Evolution of the Fgf and Fgfr gene families. Trends Genet. 2004; 20:563-569. doi: 10.1016/j.tig.2004.08.007). The binding of FGFs drives the dimerization of FGFRs; subsequently, a trans-autophosphorylation event of the intracellular kinase domain is induced, followed by the activation of downstream transduction pathways. Through triggering downstream signaling pathways, FGFRs participate in various vital physiological processes, such as proliferation, differentiation, cell migration and survival (Ornitz D. M., Itoh N. The Fibroblast Growth Factor signaling pathway. Wiley Interdiscip. Rev. Dev. Biol. 2015; 4:215-266. doi: 10.1002/wdev.176).

The catalytic activity of the kinase domain is precisely controlled. There are two general conformations for all protein kinases, including those of the FGFR family. Activation typically involves changes in the orientation of the αC-helix in the small lobe and the activation loop in the C-lobe. During the catalytic cycle, the active kinase toggles between open and closed conformations. In the open form, the kinase binds MgATP and the protein substrate, while during catalysis, the kinase adopts the closed form. Once catalysis is completed, the MgADP and phosphorylated substrate are released, and the enzyme recovers to the open conformation, preparing for the next catalytic cycle (Farrell B., Breeze A. L. Structure, activation and dysregulation of fibroblast growth factor receptor kinases: Perspectives for clinical targeting. Biochem. Soc. Trans. 2018; 46:1753-1770. doi: 10.1042/BST20180004).

Aberrant expression of FGFRs has been shown in various kinds of solid tumors, and moreover, the aberrancy is considered an oncogenic signaling pathway (Turner N., Grose R. Fibroblast growth factor signalling: From development to cancer. Nat. Rev. Cancer. 2010; 10:116-129. doi: 10.1038/nrc2780).

The compositions and treatments described herein are useful for the treatment of a host with a cancer having a dysregulation of fibroblast growth factor receptor (FGFR) pathway signaling due to an FGFR aberrancy or FGF aberrancy by administering a CDK4/6 inhibitor described herein in combination or alteration with an FGFR inhibitor, including, but not limited to, a selective FGFR inhibitor. Dysregulation of fibroblast growth factor receptor (FGFR) pathway signaling is an emerging focus for targeted therapy across multiple types of cancer, particularly, but not limited to, urothelial carcinoma, breast cancer, non-small cell lung cancer including squamous cell lung cancer and large cell carcinoma, gastric cancer including gastric adenocarcinoma, and intrahepatic cholangiocarcinoma. Dysregulation of FGFR signaling encompasses a range of FGFR family abnormalities, including, but not limited to, FGFR gene amplification, FGFR overexpression, FGFR fusions, FGFR point mutations, and FGFR gene rearrangements, as well as FGF aberrancy, including but not limited to FGF overexpression or amplification, and FGF mutations. In some embodiments, provided herein are compositions and treatments for treating a cancer with an FGFR dysregulated signaling or aberration, wherein the treatment comprises administering to the host a CDK4/6 inhibitor described herein in combination or alteration with an FGFR inhibitor, wherein the dysregulation of FGFR signaling is caused by one or more of an FGFR gene amplification, an FGFR overexpression, an FGFR fusion, an FGFR point mutation, and an FGFR gene rearrangement or an FGF mutation, overexpression, or amplification. In some embodiments, the cancer is advanced or has become metastatic.

Studies using next-generation sequencing (NGS) on samples from about 5,000 patients with various cancers showed FGFR aberrations in 7.1% of samples. FGFR overexpression accounted for the majority of these dysregulations (66%), followed by FGFR-activating mutations (26%), and FGFR gene rearrangements or fusions (8%) (Helsten T, Elkin S, Arthur E, Tomson B N, Carter J, Kurzrock R. The FGFR landscape in cancer: analysis of 4,853 tumors by next-generation sequencing. Clin Cancer Res. 2016; 22(1):259-67. doi: 10.1158/1078-0432.CCR-14-3212). Overall, dysregulation in FGFR1/2/3/4 signaling were most frequently found in urothelial (31.7%), breast (17.4%), endometrial (11.3%), and ovarian cancers (8.6%) (Helsten T, Elkin S, Arthur E, Tomson B N, Carter J, Kurzrock R. The FGFR landscape in cancer: analysis of 4,853 tumors by next-generation sequencing. Clin Cancer Res. 2016; 22(1):259-67. doi: 10.1158/1078-0432.CCR-14-3212). Overexpression of FGFRs may lead to ligand-independent FGFR signaling and is mainly caused by focal amplifications.

Study findings show a variation in the type of dysregulation and the specific gene within the family across cancer types. Over-expression or amplification accounts for approximately 89% of all FGFR1 aberrations (Helsten T, Elkin S, Arthur E, Tomson B N, Carter J, Kurzrock R. The FGFR landscape in cancer: analysis of 4,853 tumors by next-generation sequencing. Clin Cancer Res. 2016; 22(1):259-67. doi: 10.1158/1078-0432.CCR-14-3212) and has been demonstrated in approximately 16% of non-small cell lung cancer (NSCLC), including squamous cell lung cancer and large cell lung cancer, (Yang W, Yao Y W, Zeng J L, et al. Prognostic value of FGFR1 gene copy number in patients with non-small cell lung cancer: a meta-analysis. J Thorac Dis. 2014; 6 (6): 803-9. doi: 10.3978/j.issn.2072-1439.2014.05.02); (Weiss J, Sos M L, Seidel D, et al. Frequent and focal FGFR1 amplification associates with therapeutically tractable FGFR1 dependency in squamous cell lung cancer. Sci Transl Med. 2010; 2(62):62ra93. doi: 10.1126/scitranslmed.3001451), 6% of small cell lung carcinomas, (Peifer M, Fernandez-Cuesta L, Sos M L, et al. Integrative genome analyses identify key somatic driver mutations of small-cell lung cancer. Nat Genet. 2012; 44 (10): 1104-1110. doi: 10.1038/ng.2396).

FGFR1 amplification has also been found in approximately 18% of osteosarcoma, and is associated with sensitivity to FGFR inhibitors in preclinical in vivo models. In breast cancer, amplification of FGFR1- and/or 11q12-14 (which contains CCND1, FGF3, FGF4, and FGF19) have been observed in 23% of hormone receptor-positive (HR+), 27% of HER2-amplified, and 7% of triple-negative cases and is predictive for early relapses and poor outcome. Many FGFR1-amplified breast cancer cell lines are addicted to FGFR1 amplification and FGFR1 amplification also drives resistance to endocrine therapy.

FGFR2 amplification was demonstrated in approximately 4% of gastric cancers (Matsumoto K, Arao T, Hamaguchi T, et al. FGFR2 gene amplification and clinicopathological features in gastric cancer. Br J Cancer. 2012; 106 (4): 727-732. doi: 10.1038/bjc.2011.603). However, gastric and breast cancer cell lines with FGFR2 amplifications were particularly sensitive to selective FGFR inhibitors, suggesting that the FGFR2 amplification confers addiction to the FGFR signaling pathway (Pearson A, Smyth E, Babina I S, et al. High-level clonal FGFR amplification and response to FGFR inhibition in a translational clinical trial. Cancer Discov. 2016; 6 (8): 838-851. doi: 10.1158/2159-8290.CD-15-1246; Campbell J, Ryan C J, Brough R, et al. Large-scale profiling of kinase dependencies in cancer cell lines. Cell Rep. 2016; 14 (10): 2490-2501. doi: 10.1016/j.celrep.2016.02.023). FGFR2 amplification is associated with the maintenance of tumor-initiating cells, poorer prognosis, and high sensitivity to FGFR inhibitors.

FGFR3 amplification is relatively uncommon but was demonstrated in 3% of urothelial cancers (Helsten T, Elkin S, Arthur E, Tomson B N, Carter J, Kurzrock R. The FGFR landscape in cancer: analysis of 4,853 tumors by next-generation sequencing. Clin Cancer Res. 2016; 22(1):259-67. doi: 10.1158/1078-0432.CCR-14-3212).

Activating mutations in FGFRs may result in aberrant FGFR signaling through multiple mechanisms, including the following: (i) enhanced activation of the kinase domain; (ii) ligand-independent dimerization of the receptors; and (iii) altered affinity for FGF ligands.

Activating mutations in FGFR2 occur in 12% to 14% of endometrial cancers and have been demonstrated in a small proportion of squamous NSCLCs, gastric cancers, and urothelial cancers (Helsten T, Elkin S, Arthur E, Tomson B N, Carter J, Kurzrock R. The FGFR landscape in cancer: analysis of 4,853 tumors by next-generation sequencing. Clin Cancer Res. 2016; 22(1):259-67. doi: 10.1158/1078-0432.CCR-14-3212); (Touat M, Ileana E, Postel-Vinay S, Andre F, Soria J C. Targeting FGFR signaling in cancer. Clin Cancer Res. 2015; 21(12): 2684-2694. doi: 10.1158/1078-0432.CCR-14-2329). FGFR2 mutations in, e.g., endometrial cancer, mainly consist of missense activating mutations of the extracellular domain (S252W, P253R). In vitro and in vivo models demonstrated the selective sensitivity of FGFR2-mutant endometrial cancer to FGFR inhibitors.

Activating mutations in FGFR3 are particularly prevalent in urothelial cancers, occurring in up to 80% of non-muscle invasive urothelial cell carcinomas, 20% of high-grade invasive urothelial cancers, and 5% of cervical cancers (Touat M, Ileana E, Postel-Vinay S, Andre F, Soria J C. Targeting FGFR signaling in cancer. Clin Cancer Res. 2015; 21(12): 2684-2694. doi: 10.1158/1078-0432.CCR-14-2329). Urothelial bladder carcinoma has the most established association with altered FGFR signaling, with up to 80% of low-grade tumors harboring FGFR mutations and compelling in vivo and in vitro data. Comprehensive molecular characterization of this cancer revealed a cluster of tumors with papillary morphology characterized by a high rate of molecular alterations of FGFR3 (mutations, copy number gain, fusions), which may have some degree of FGFR addiction. The most common activating mutations affect either the extracellular (R248C, S249C) or the transmembrane (G370C, S371C, Y373C, G380R, A391E) domains of the protein. Kinase domain mutations (N540S, K650E, K650M, K650N, K650Q, and K650T) are rarer.

Activating mutations in FGFR1 and FGFR4 are relatively uncommon and have been observed in pilocytic astrocytoma (FGFR1) and rhabdomyosarcoma (FGFR4) (Touat M, Ileana E, Postel-Vinay S, Andre F, Soria J C. Targeting FGFR signaling in cancer. Clin Cancer Res. 2015; 21(12): 2684-2694. doi: 10.1158/1078-0432.CCR-14-2329). Activating mutations of FGFR4 (affecting the kinase domain) are found in 6% to 8% of patients with rhabdomyosarcoma. In a comprehensive genomic analysis of 147 cases of rhabdomyosarcoma, FGFR signaling was the most significantly altered pathway in both fusion-positive and fusion-negative rhabdomyosarcomas. Cell lines and explants harboring FGFR4-activating mutations were both sensitive to FGFR inhibitors.

Fusion genes are hybrid genes formed by the rearrangement of two previously independent genes. They can occur as a result of translocation, chromosomal inversion, duplication, or deletion. Several fusion proteins are known to play crucial roles in the initiation and progression of cancer, thereby representing ideal targets for rational drug design strategies.

Many recent efforts of molecular screening programs and precision medicine have allowed the identification of multiple fusion genes between FGFR1, -2, and -3 and multiple partners (including TACC1, TACC3, BAIAP2L1, BICC1, CASP7, and AHCYL1) in several malignancies such as glioblastoma, urothelial bladder carcinoma, non-small cell lung cancer (NSCLC), and cholangiocarcinoma.

In intrahepatic cholangiocarcinoma, FGFR2 fusions/translocations with either AHCYL1 or BICC1 have been described in 13.6% of cases and are mutually exclusive with KRAS/BRAF mutations. In vivo models demonstrated the transforming potential of these alterations, and high sensitivity to FGFR inhibitors.

FGFR2 fusions/translocations are found in approximately 14% of intrahepatic cholangiocarcinomas and occur occasionally in lung, thyroid, and prostate cancer (Touat M, Ileana E, Postel-Vinay S, Andre F, Soria J C. Targeting FGFR signaling in cancer. Clin Cancer Res. 2015; 21(12): 2684-2694. doi: 10.1158/1078-0432.CCR-14-2329); (Arai Y, Totoki Y, Hosoda F, et al. Fibroblast growth factor receptor 2 tyrosine kinase fusions define a unique molecular subtype of cholangiocarcinoma. Hepatology. 2014; 59 (4): 1427-1434. doi: 10.1002/hep.26890); (Wu Y M, Su F, Kalyana-Sundaram S, et al. Identification of targetable FGFR gene fusions in diverse cancers. Cancer Discov. 2013; 3 (6): 636-647. doi: 10.1158/2159-8290.CD-13-0050). FGFR1 translocations are relatively uncommon but have been observed in glioblastoma, breast cancer, squamous cell lung carcinoma, and myeloproliferative syndrome (Touat M, Ileana E, Postel-Vinay S, Andre F, Soria J C. Targeting FGFR signaling in cancer. Clin Cancer Res. 2015; 21(12): 2684-2694. doi: 10.1158/1078-0432.CCR-14-2329).

FGFR3 translocations/fusions account for 15% to 20% of multiple myelomas and have been observed in glioblastoma and bladder cancer (Touat M, Ileana E, Postel-Vinay S, Andre F, Soria J C. Targeting FGFR signaling in cancer. Clin Cancer Res. 2015; 21(12): 2684-2694. doi: 10.1158/1078-0432.CCR-14-2329).

Fusions involving FGFR3 and TACC3 (transforming acidic coiled-coil containing protein 3) are found in 3% to 7% of glioblastomas, 3% to 6% of urothelial bladder carcinomas, and other tumor types at lower frequencies. In mouse xenograft models, the induction of FGFR3-TACC3 expression in human astrocytes resulted in the development of glioma-like tumors. In vivo, both FGFR3-TACC3-initiated bladder carcinoma and glioblastoma were extremely sensitive to specific FGFR inhibitors, suggesting oncogenic addiction to the fusion protein. FGFR gene fusions that have been associated with the development of cancer include, but not limited to, FGFR3-TACC3, FGFR3-BAIAP2L1, FGFR2-BICC1, and FGFR2-CASP7.

FGF aberrancy has also been shown to play a role in the development in cancers with dysregulated FGFR signaling. For example, amplification of FGF19 was found to be significantly associated with increased risks of hepatocellular carcinoma (HCC) (see Raja, FGF19-FGFR4 Signaling in Hepatocellular Carcinoma. Cells. 2019 June; 8(6): 536).

Determining the mutational status of FGFR-mutant cancer is well known in the art. For example, The Therascreen FGFR RGQ RT-PCR Kit is a real-time, reverse transcription PCR test for the qualitative detection of two point mutations in exon 7 [p.R248C (c.742C>T), p.S249C (c.746C>G)], two point mutations in exon 10 [p.G370C (c.1108G>T) and p.Y373C (c.1118A>G)] and two fusions (FGFR3-TACC3v1 and FGFR3-TACC3v3) in the fibroblast growth factor receptor 3 (FGFR3) gene in RNA samples derived from formalin-fixed paraffin-embedded (FFPE) urothelial tumor tissue. The test is indicated for use as an aid in identifying urothelial cancer (UC) patients who harbor these alterations and are therefore eligible for treatment with BALVERSA (erdafitinib). Specimens are processed using the RNeasy DSP FFPE Kit for manual sample preparation followed by reverse transcription and then automated amplification and detection on the Rotor-Gene Q MDx (US) instrument.

In addition, direct DNA sequencing to identify mutations in the genes encoding FGFR1-4 and non-FGFR genes rendering tumors resistant to FGFR-TKIs are well known. Other useful mutational analysis techniques include, but are not limited to, analysis by dHPLC, DNA endonuclease (SURVEYOR) and HPLC, HRMA, massively parallel sequencing, TaqMan PCR, cycleave PCR, fragment analysis, mutation-specific PCR, mutant enriched PCR, ARMS, mutant enriched ARMS TaqMan PCR, PCR-invader, PCR-RFLP, and others.

Plasma cell-free tumor DNA, or circulating tumor DNA (ctDNA), from liquid biopsy is a potential source of tumor genetic material, in the absence of tissue biopsy, for FGFR-mutational testing. Allele specific PCR, Scorpion Amplified Refractory Mutation System (ARMS) PCR, droplet digital PCR (ddPCR), and next generation sequencing (NGS) are the most commonly used technologies for mutation detection in ctDNA, and are generally known in the art. See Veldore et al., Lung Cancer (Auckl). 2018; 9: 1-11; Bordi et al., Transl Lung Cancer Res. 2015; 4(5):584-597; Fenizia et al., Future Oncol. 2015; 11(11):1611-1623; Mao et al., Medicine. 2015; 94(21):e775. doi: 10.1097/MD.0000000000000775; Marchetti et al., J Thorac Oncol. 2015; 10(10):1437-1443; Sholl et al., Arch Pathol Lab Med. doi:10.5858/arpa.2016-0163-SA; Sorber et al., Lung Cancer. 2016 May 4. pii: S0169-5002(16)30312-9. doi: 10.1016/j.lungcan.2016.04.026; Westwood et al., Health Technol Assess. 2014; 18(32):1-166; Lindeman et al., J Thorac Oncol. 2013; 8(7):823-859; Socinski et al., Clin Lung Cancer. 2010; 11(3):149-159, all incorporated herein by reference. Determining amplification or over-expression status of FGFR is also known in the art, and commercial assays are available to determine overexpression and/or amplification status. For example, FGFR FISH tests are designed to detect amplification or translocation the FGFR. For example, an FGFR locus is reported as amplified when the ratio of FGFR to the tested locus exceeds a threshold or an average number copies of the FGFR locus are observed per tumor nucleus. See, e.g., Schildhaus H U, Heukamp L C, Merkelbach-Bruse S, et al: Definition of a fluorescence in-situ hybridization score identifies high- and low-level FGFR1 amplification types in squamous cell lung cancer. Mod Pathol 2012 November; 25(11):1473-1480; see also Liang et al. 2012 Trends Pharmacol Sci 33.

Particular cancers suitable for targeting with the compositions and treatments described herein include those with FGFR aberrations, including urothelial cancer, bladder cancer, breast cancer, endometrial cancer, ovarian cancer, osteosarcoma, carcinoma unknown primary, glioma, glioblastoma, liver cancer, including hepatocellular carcinoma (HCC) and intrahepatic cholangiocarcinoma, gastric cancer including gastric adenocarcinoma, non-small cell lung cancer, small cell lung cancer, rhabdomyosarcoma, pancreatic exocrine cancer, colorectal cancer, renal cell cancer, neuroendocrine cancer, head and neck (squamous) cancer, melanoma, leiomyosarcoma, cervical cancer, and sarcoma. In some embodiments, the cancer is advanced or metastatic.

In some embodiments, provided herein are compositions and treatments for treating a cancer with an FGFR dysregulated signaling or aberration caused by an FGFR1 aberration, wherein the treatment comprises administering to the host a CDK4/6 inhibitor described herein in combination with a selective FGFR inhibitor. In some embodiments, the FGFR inhibitor is selected from the group consisting of erdafitinib, pemigatinib, infigratinib, AZD4547, futibatinib, derazantinib, Debio1347, PRN1371, FIIN, 2, GSK3052230, and PD173074. In some embodiments, the FGFR1 aberration is an FGFR1 over-expression or amplification, FGFR1 mutation, FGFR1 translocation, or FGFR1 fusion. In some embodiments, the cancer is selected from non-small cell lung cancer, including squamous cell lung cancer, large cell lung cancer, and lung adenocarcinoma, breast cancer, including hormone receptor positive cancer such as estrogen-receptor positive breast cancer, HER2-positive or HER2-amplified breast cancer, osteosarcoma, pilocytic astrocytoma, and glioblastoma. In some embodiments, the cancer is non-small cell lung cancer and has an FGFR1-amplification. In some embodiments, the cancer is small cell lung cancer and has an FGFR1-amplification. In some embodiments, the cancer is ER+, HER2+, HER2-amplified breast cancer and has an FGFR1-amplification. In some embodiments, the cancer is triple negative breast cancer and has an FGFR1-amplification. In some embodiments, the cancer is an osteosarcoma and has an FGFR1-amplification. In some embodiments, the cancer is pilocytic astrocytoma and has an FGFR mutation. In some embodiments, the cancer is glioblastoma, non-small cell lung cancer (NSCLC), and cholangiocarcinoma and has an FGFR1 translocation or rearrangement. In some embodiments, the cancer is metastatic or advanced. In some embodiments, the CDK4/6 inhibitor administered is Compound I, Compound IA, or Compound IA, Form B. In alternative embodiments, the CDK4/6 inhibitor is Compound III. In some embodiments, the CDK4/6 inhibitor administered is Compound VI.

In some embodiments, provided herein are compositions and treatments for treating a cancer with an FGFR dysregulated signaling or aberration caused by an FGFR2 aberration, wherein the treatment comprises administering to the host a CDK4/6 inhibitor described herein in combination with a selective FGFR inhibitor. In some embodiments, the FGFR inhibitor is selected from the group consisting of erdafitinib, pemigatinib, infigratinib, AZD4547, futibatinib, derazantinib, LY287445, Debio1347, PRN1371, alofanib, bemarituzumab, and FIIN-2. In some embodiments, the FGFR2 aberration is an FGFR2 over-expression or amplification, FGFR2 mutation, or an FGFR2 translocation or fusion. In some embodiments, the cancer is selected from gastric cancer, breast cancer including, but not limited to, hormone receptor positive cancer such as estrogen-receptor positive breast cancer, HER2-positive or HER2-amplified breast cancer, for example ER+/HER2-amplified breast cancer, endometrial cancer, non-small cell lung cancer, including squamous cell lung cancer, gastric cancer, urothelial cancer, intrahepatic cholangiocarcinoma, thyroid cancer, and prostate cancer. In some embodiments, the cancer is gastric cancer, for example, gastric adenocarcinoma, and the cancer has an FGFR2 amplification. In some embodiments, the cancer is endometrial cancer, a non-small cell lung cancer, or a gastric adenocarcinoma, and the cancer has an FGFR2 mutation. In some embodiments, the FGFR2 mutation is selected from an S252W substitution and a P253R substitution. In some embodiments the cancer is intrahepatic cholangiocarcinoma, non-small cell lung cancer, or thyroid cancer and the cancer has an FGFR2 fusion. In some embodiments, the FGFR2 fusion is a FGFR2-BICC1, FGFR2-AHCYL1 (adenosyl homocysteinase like 1) fusion, or FGFR2-CASP7 (caspase 7) fusion. In some embodiments, the CDK4/6 inhibitor administered is Compound I, Compound IA, or Compound IA, Form B. In alternative embodiments, the CDK4/6 inhibitor is Compound III. In some embodiments, the CDK4/6 inhibitor administered is Compound VI.

In some embodiments, provided herein are compositions and treatments for treating a cancer with an FGFR dysregulated signaling or aberration caused by an FGFR3 aberration, wherein the treatment comprises administering to the host a CDK4/6 inhibitor described herein in combination with a selective FGFR inhibitor. In some embodiments, the FGFR inhibitor is selected from the group consisting of erdafitinib, pemigatinib, infigratinib, AZD4547, futibatinib, derazantinib, LY287445, Debio1347, PRN1371, MGFR1877S, vofatamab, and FIIN-2. In some embodiments, the FGFR3 aberration is an FGFR3 over-expression or amplification, FGFR3 mutation, FGFR3 translocation, or FGFR3 fusion. In some embodiments, the cancer is selected from cervical cancer, urothelial cancer, glioblastoma, and multiple myeloma. In some embodiments, the cancer is a cervical cancer, and the cancer has a FGFR3 mutation. In some embodiments, the FGFR3 mutation is selected from one of the following substitutions: R248C, S249C, G370C, S371C, Y373C, G380R, A391E, N540S, K650E, K650M, K650N, K650Q, and K650T. In some embodiments, the cancer is glioblastoma, or multiple myeloma, and the cancer has an FGR translocation or FGFR fusion. In some embodiments, the FGFR3 fusion is a FGFR3 and TACC3 (transforming acidic coiled-coil containing protein 3) fusion or a FGFR3-BAIAP2L1 (BAI1-associated protein 2-like 1fusion) fusion. In some embodiments, the cancer is advanced or metastatic. In some embodiments, the CDK4/6 inhibitor administered is Compound I, Compound IA, or Compound IA, Form B. In alternative embodiments, the CDK4/6 inhibitor is Compound III. In some embodiments, the CDK4/6 inhibitor administered is Compound VI.

In some embodiments, provided herein are compositions and treatments for treating a cancer with an FGFR dysregulated signaling or aberration caused by an FGFR4 or FGF19 aberration, wherein the treatment comprises administering to the host a CDK4/6 inhibitor described herein in combination with a selective FGFR inhibitor. In some embodiments, the FGFR inhibitor is selected from the group consisting of infigratinib, AZD4547, futibatinib, derazantinib, LY287445, INCB062079, BLU9931, H3-6527, fisogatinib, Debio1347, PRN1371, roblitinib, and FIIN-2. In some embodiments, the FGFR4 aberration is an FGFR4 over-expression or amplification, FGFR4 mutation, FGFR4 translocation, or FGFR4 fusion. In some embodiments, the cancer is selected from liver cancer, including hepatocellular carcinoma, rhabdomyosarcoma, breast cancer, including hormone ER+/HER2-positive or HER2-amplified breast cancer, endometrial cancer, and ovarian cancer. In some embodiments, the cancer is rhabdomyosarcoma and the cancer has an FGFR4 mutation. In some embodiments, the cancer is hepatocellular carcinoma and the cancer has aberrant fibroblast growth factor 19 (FGF19) signaling (FGF19-positive) through FGFR4. In some embodiments, the cancer has an overexpressed or amplification of FGF19. In some embodiments, the cancer is a hepatocellular carcinoma (HCC) and has an overexpressed or amplification of FGF19. In some embodiments, the cancer is advanced or metastatic. In some embodiments, the CDK4/6 inhibitor administered is Compound I, Compound IA, or Compound IA, Form B. In alternative embodiments, the CDK4/6 inhibitor is Compound III.

In some embodiments, the cancer to be treated is CDK4/6 replication dependent. Cancers that are CDK4/6 replication dependent require the activity of CDK4/6 for replication or proliferation. CDK 4/6 replication dependent cancers generally have an intact and function Rb pathway and/increased expression of CDK4/6 activators (cyclin D), and/or a d-type cyclin activating features (DCAF)—including CCND1 translocation, CCND1-3 3′UTR loss, and amplification of CCND2 or CCND3 (see Gong et al. Genomic aberrations that activate D-type cyclins are associated with enhanced sensitivity to the CDK4 and CDK5 inhibitor abemaciclib. Cancer Cell. 2017; 32(6):761-76). Tumors that are wildtype for RB and CCNE1/2 as well as have one of the DCAF described above are generally classified as “CDK4/6 dependent”.

In any of the embodiments above, the cancer has progressed during or following at least one line of prior chemotherapy, for example a platinum-containing chemotherapy such as cisplatin, carboplatin, oxaliplatin, nedaplatin, triplatin tetranitrate, phenanthriplatin, picoplatin, or satraplatin.

In some embodiments of the above, the cancer to be treated does not have a mutually exclusive mutation to the FGFR mutation, for example, a KRAS mutation or BRAF mutation. In some embodiments of the above, the host is administered an additional anti-cancer active agent. In some embodiments, the additional active agent is an immune modulator or checkpoint inhibitor. In one aspect of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, TIGIT inhibitor, Siglec-15 inhibitor, B7-H3 (CD272) inhibitor, BTLA inhibitor (CD272), small molecule, peptide, nucleotide, or other inhibitor. In certain aspects, the immune modulator is an antibody, such as a monoclonal antibody.

In some embodiments, the immune checkpoint inhibitor is a PD-1 inhibitor that blocks the interaction of PD-1 and PD-L1 by binding to the PD-1 receptor, and in turn inhibits immune suppression. In some embodiments, the immune checkpoint inhibitor is a PD-1 immune checkpoint inhibitor selected from, but not limited to, nivolumab (Opdivo®), pembrolizumab (Keytruda®), pidilizumab (Medivation), AMP-224 (Amplimmune); sasanlimab (PF-06801591; Pfizer), spartalizumab (PDR001; Novartis), cemiplimab (Libtayo®; REGN2810; Regeneron), retifanlimab (MGA012; MacroGenics), tislelizumab (BGB-A317; BeiGene), camrelizumab (SHR-1210; Jiangsu Hengrui Medicine Company and Incyte Corporation), and dostarlimab (TSR-042; Tesaro).

In some embodiments, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, atezolizumab (Tecentriq®, Genentech), durvalumab (Imfinzi®, AstraZeneca); avelumab (Bavencio®; Merck), envafolimab (KN035; Alphamab), BMS-936559 (Bristol-Myers Squibb), lodapolimab (LY3300054; Eli Lilly), cosibelimab (CK-301; Checkpoint Therapeutics), sugemalimab (CS-1001; Cstone Pharmaceuticals), adebrelimab (SHR-1316; Jiangsu HengRui Medicine), CBT-502 (CBT Pharma), and BGB-A333 (BeiGene).

In some embodiments, the immune checkpoint inhibitor is a PD-L1/VISTA inhibitor. PD-L1-VISTA inhibitors include, but are not limited to, CA-170 (Curis Inc.). In some embodiments, the immune checkpoint inhibitor is a VISTA immune checkpoint inhibitor. VISTA inhibitors include, but are not limited to, JNJ-61610588 (Johnson & Johnson).

In one aspect of this embodiment, the immune checkpoint inhibitor is a CTLA-4 immune checkpoint inhibitor that binds to CTLA-4 and inhibits immune suppression. CTLA-4 inhibitors include, but are not limited to, ipilimumab (Yervoy®, Bristol Myers Squibb); tremelimumab (AstraZeneca/MedImmune), zalifrelimab (AGEN1884; Agenus) and AGEN2041 (Agenus).

In another embodiment, the immune checkpoint inhibitor is a LAG-3 immune checkpoint inhibitor. Examples of LAG-3 immune checkpoint inhibitors include, but are not limited to, relatlimab (BMS-986016; Bristol-Myers Squibb), GSK2831781 (GlaxoSmithKline), eftilagimod alpha (IMP321; Prima BioMed), leramilimab (LAG525; Novartis), MK-4280 (Merck), REGN3767 (Regeneron), TSR-033 (Tesaro), BI754111 (Bohringer Ingelheim), Sym022 (Symphogen). the dual PD-1 and LAG-3 inhibitor tebotelimab (MGD013; MacroGenics), and the dual PD-L1 and LAG-3 inhibitor FS118 (F-Star).

In yet another aspect of this embodiment, the immune checkpoint inhibitor is a TIM-3 immune checkpoint inhibitor. TIM-3 inhibitors include, but are not limited to, TSR-022 (Tesaro), MBG453 (Novartis), Sym023 (Symphogen), INCAGN2390 (Incyte), LY3321367 (Eli Lilly and Company), BMS-986258 (BMS), SHR-1702 (Jiangsu HengRui), and RO7121661 (Roche).

In yet another aspect of this embodiment, the immune checkpoint inhibitor is a TIGIT (T cell immunoreceptor with Ig and ITIM domains) immune checkpoint inhibitor. TIGIT immune checkpoint inhibitors include, but are not limited to, MK-7684 (Merck), Etigilimab/OMP-313 M32 (OncoMed), Tiragolumab/MITIG7192A/RG-6058 (Genentech), BMS-986207 (BMS), AB-154 (Arcus Biosciences), and ASP-8374 (Potenza).

Other immune checkpoint inhibitors for use in the invention described herein include, but are not limited to, B7-H3/CD276 immune checkpoint inhibitors such as enoblituzumab (MGA217, Macrogenics) MGD009 (Macrogenics), ¹³¹I-8H9/omburtamab (Y-mabs), and I-8H9/omburtamab (Y-mabs), indoleamine 2,3-dioxygenase (IDO) immune checkpoint inhibitors such as Indoximod and INCB024360, killer immunoglobulin-like receptors (KIRs) immune checkpoint inhibitors such as Lirilumab (BMS-986015), carcinoembryonic antigen cell adhesion molecule (CEACAM) inhibitors (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or cross-reacts with CEACAM-1 and CEACAM-5 as described in, e.g., WO 2013/054331 and US 2014/0271618.

Still other checkpoint inhibitors can be molecules directed to B and T lymphocyte attenuator molecule (BTLA), for example as described in Zhang et al., Monoclonal antibodies to B and T lymphocyte attenuator (BTLA) have no effect on in vitro B cell proliferation and act to inhibit in vitro T cell proliferation when presented in a cis, but not trans, format relative to the activating stimulus, Clin Exp Immunol. 2011 January; 163(1): 77-87, and TAB004/JS004 (Junshi Biosciences).

In another embodiment, the immune checkpoint inhibitor is a sialic acid-binding immunoglobulin-like lectin 15 (Siglec-15) inhibitor, including, but not limited to, NC318 (an anti-Siglec-15 mAb).

### Pharmaceutical Compositions and Dosage Forms

Any of the compounds for use in the compositions and treatments as disclosed herein can be administered as the neat chemical, but are more typically administered as a pharmaceutical composition, that includes an effective amount for a host, typically a human, in need of such treatment for any of the disorders described herein. Accordingly, the disclosure provides pharmaceutical compositions for use in the methods described herein comprising an effective amount of compound or pharmaceutically acceptable salt together with at least one pharmaceutically acceptable carrier for any of the uses described herein. The pharmaceutical composition may contain a compound or salt as the only active agent, or, in an alternative embodiment, the compound and at least one additional active agent.

In certain embodiments the pharmaceutical composition is in a dosage form that contains from about 0.0005 mg to about 2000 mg, from about 0.001 mg to about 1000 mg, from about 0.001 mg to about 600 mg, or from about 0.001 mg to about 1, 5, 10, 15, 20, 25, 50, 100, 200 or 300 mg mg of the active compound. In another embodiment the pharmaceutical composition is in a dosage form that contains from about 0.01 mg to about 1, 5, 10, 15, 20, 25, 50 or 100 mg, from about 0.05 mg to about 1, 5, 10, 15, 20, 25, 50 or 100 mg, from about 0.1 mg to about 1, 5, 10, 15, 20, 25 or 50 mg, from about 0.02 mg to about 1, 5, 10, 15, 20, 25 or 50 mg of the active compound, from about 0.5 mg to about 1, 5, 10, 15, 20, 25 or 50 mg. In another embodiment the pharmaceutical composition is in a dosage form that contains from about 0.01 mg to about 10 mg, from about 0.05 mg to about 8 mg, or from about 0.05 mg to about 6 mg, or from about 0.05 mg to about 5 mg of the active compound. In another embodiment the pharmaceutical composition is in a dosage form that contains from about 0.1 mg to about 10 mg, from about 0.5 mg to about 8 mg, or from about 0.5 mg to about 6 mg, or from about 0.5 mg to about 5 mg of the active compound. Nonlimiting examples are dosage forms with at least about 0.0005, 0.001, 0.01, 0.1, 1, 2.5, 5, 10, 25, 50, 100, 200, 250, 300, 400, 500, 600, 700, or 750 mg of active compound, or its salt. Alternative nonlimiting examples are dosage forms with not greater than about 0.01, 0.1, 1, 2.5, 5, 10, 25, 50, 100, 200, 250, 300, 400, 500, 600, 700, or 750 mg of active compound, or its salt.

In some embodiments, one or more of the compounds disclosed herein for use as described are administered once a day (QD), twice a day (BID), or three times a day (TID). In some embodiments, compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer, or in an alternative schedule as described herein.

In certain embodiments, one or more of the compounds for use herein are administered once a day, twice a day, three times a day, or four times a day.

In certain embodiments, one or more of the compounds for use are described herein are administered orally once a day. In certain embodiments, one or more of the compounds for use are described herein are administered orally twice a day. In certain embodiments, one or more of the compounds for use are described herein are administered orally three times a day. In certain embodiments, one or more of the compounds for use are described herein are administered orally four times a day.

In certain embodiments one or more of the compounds for use are described herein are administered intravenously once a day. In certain embodiments one or more of the compounds for use as described herein are administered intravenously twice a day. In certain embodiments, one or more of the compounds for use as described herein are administered intravenously three times a day. In certain embodiments one or more of the compounds for use as described herein are administered intravenously four times a day.

In some embodiments one or more of the compounds for use as described herein are administered with a treatment holiday in between treatment cycles. For example, one or more of the compounds may have a treatment holiday of 1 day, 2 days, 3 days, 4 days, 5 days, 6 days, 7 days, 8 days, 9 days, 10 days, 11 days, 12 days, 13 days, or 14 days per treatment cycle.

In other aspects, this invention provides the administration of a pharmaceutical composition comprising a therapeutically effective amount of a selective CDK4/6 inhibitor selected from Compound I, Compound II, Compound III, Compound IV, Compound V, Compound VI, or a pharmaceutically acceptable salt thereof, and the administration of a pharmaceutical composition comprising an effective amount of an FGFR inhibitor, or a pharmaceutically acceptable salt thereof, and one or more pharmaceutically acceptable diluents, preservatives, solubilizers, emulsifiers, adjuvants, excipients, or carriers. Such excipients include liquids such as water, saline, glycerol, polyethylene glycol, hyaluronic acid, ethanol, and the like.

In some embodiments, provided herein is a pharmaceutical composition comprising a therapeutically effective amount of a selective CDK4/6 inhibitor selected from Compound I, Compound II, Compound III, Compound IV, Compound V, Compound VI, or a pharmaceutically acceptable salt thereof, and an effective amount of an FGFR inhibitor, or a pharmaceutically acceptable salt thereof, and one or more pharmaceutically acceptable diluents, preservatives, solubilizers, emulsifiers, adjuvants, excipients, or carriers. In some embodiments, the FGFR inhibitor is selected from the group consisting of erdafitinib, infigratinib, pemigatinib, AZD4547, futibatinib (TAS-120), derazantinib, roblitinib, LY287445, INCB062079, BLU9931, PRN1371, FIIN-2, PD173074, H3B-6527, fisogatinib, alofanib, and Debio1347. The pharmaceutical composition may include a molar ratio of the CDK4/6 inhibitor and FGFR inhibitor. In non-limiting illustrative embodiments, the pharmaceutical composition may contain a molar ratio of about up to 0.5:1, about up to 1:1, about up to 2:1, about up to 3:1 or from about up to 1.5:1 to about up to 4:1 of the CDk4/6 inhibitor to the FGFR inhibitor.

Suitable excipients for non-liquid formulations are also known to those of skill in the art. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the patient, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the compositions of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in an acceptably nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacterium retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

A pharmaceutically or therapeutically effective amount of each composition will be delivered to the host. The precise effective amount will vary from host to host and will depend upon the species, age, the patient's size and health, the nature and extent of the condition being treated, recommendations of the treating physician, and the therapeutics or combination of therapeutics selected for administration. the effective amount for a given situation can be determined by routine experimentation. For purposes of the disclosure, a therapeutic amount may for example be in the range of about 0.01 mg/kg to about 250 mg/kg body weight, more preferably about 0.1 mg/kg to about 10 mg/kg, in at least one dose. The host can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The therapeutically effective dosage of any active compound described herein will be determined by the health care practitioner depending on the condition, size and age of the patient as well as the route of delivery. In one non-limited embodiment, a dosage from about 0.1 to about 200 mg/kg has therapeutic efficacy, with all weights being calculated based upon the weight of the active compound, including the cases where a salt is employed. In some embodiments, the dosage may be the amount of compound needed to provide a serum concentration of the active compound of up to about 10 nM, 50 nM, 100 nM, 200 nM, 300 nM, 400 nM, 500 nM, 600 nM, 700 nM, 800 nM, 900 nM, 1 μM, 5 μM, 10 μM, 20 μM, 30 μM, or 40 μM.

In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form, or as otherwise described herein. Examples of dosage forms with at least 1, 2, 3, 4, 5, 10, 15, 20, 25, 50, 100, 150, 200, 250, 300, 400, 500, 600, 700, or 750 mg of active compound, or its salt.

The pharmaceutical preparations are preferably in unit dosage forms. In such form, the preparation can be subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The methods as disclosed herein provide for the administration of a CDK4/6 inhibitor described herein and administration of an FGFR inhibitor described herein. In some embodiments, the CDK4/6 inhibitor is administered at least once a day on a continuous administration schedule, that is, for example, at least 7 days, at least 14 days, at least 21 days, at least 28 days, at least 35 days, at least 42 days, at least 56 days or more, and the FGFR inhibitor is also administered at least once a day on a continuous administration schedule, for example, at least 7 days, at least 14 days, at least 21 days, at least 28 days, at least 35 days, at least 42 days, at least 56 days or more.

In some embodiments, the FGFR inhibitor is administered only for a partial period during each cycle. For example, in some embodiments, the FGFR inhibitor is administered at least once a day for the first 5 days of a 7-day cycle, the first 14 days of a 21-day cycle, the first 21 days of a 28-day cycle, and the CDK4/6 inhibitor is administered on the same schedule, wherein the cycle is repeated one or more times, for example, 2, 3, 4, 5, 6, 7, 8, 9, 10, 11, or 12 times or more. Alternatively, the FGFR inhibitor is administered for a partial period during each cycle, for example at least once a day on the first 5 days of a 7-day cycle, the first 14 days of a 21-day cycle, the first 21 days of a 28-day cycle, and the CDK4/6 inhibitor is administered at least once a day on each day of the cycle, for example for 7 days of a 7-day cycle, for 14 days of 14-day cycle, for 21 days of a 21-day cycle, or for 28 days of a 28-day cycle, wherein the cycle is repeated one or more time, for example, 2, 3, 4, 5, 6, 7, 8, 9, 10, 11, or 12 times of more.

In still further alternative embodiments, the FGFR inhibitor may be administered one time, two times, or three times during a cycle, for example once a week during a 21-day cycle, and the CDK4/6 inhibitor is administered at least once a day during each day of the cycle.

In yet further alternative embodiments, the FGFR inhibitor is administered on a set administration schedule, for example at least once a day on the first 5 days of a 7-day cycle, the first 14 days of a 21-day cycle, the first 21 days of a 28-day cycle, or each day of a 28-day cycle, and the CDK4/6 inhibitor is administered intermittently, for example, once every other day, three times a week, once every week, once every two weeks, once every three weeks, or once every 28 days, wherein the cycle is repeated one or more times, for example, 2, 3, 4, 5, 6, 7, 8, 9, 10, 11, or 12 times.

In some alternative embodiments, the CDK4/6 inhibitor is administered at least every day that the FGFR inhibitor is administered.

### EMBODIMENTS

The application of the compositions and methods described herein include at least the following:
1. A composition or medicament for use in, or treatment for, treating a host with a non-small cell lung cancer having a dysregulated fibroblast growth factor receptor (FGFR) signaling pathway caused by an FGFR1 or FGFR2 aberration wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is selected from Compound I-VI or a pharmaceutically acceptable salt thereof.
2. The composition or medicament or treatment of embodiment 1, wherein the non-small cell lung cancer has an FGFR1 aberration.
3. The composition or medicament or treatment of any of embodiments 1 or 2, wherein the FGFR1 aberration is a result of an FGFR1 mutation, FGFR1 overexpression or amplification, or FGFR1 translocation or fusion.
4. The composition or medicament or treatment of embodiment 2, wherein the FGFR1 aberration is an FGFR1 overexpression or amplification.
5. The composition or medicament or treatment of embodiment 1, wherein the non-small cell lung cancer has an FGFR2 aberration.
6. The composition or medicament or treatment of any of embodiments 1 or 5, wherein the FGFR2 aberration is a result of an FGFR2 mutation, FGFR2 overexpression or amplification, or FGFR2 translocation or fusion.
7. The composition or medicament or treatment of embodiment 6, wherein the FGFR2 aberration is an FGFR1 overexpression or amplification.
8. The composition or medicament or treatment of any of embodiments 1 to 7, wherein the non-small cell lung cancer is large cell lung carcinoma.
9. The composition or medicament or treatment of any of embodiments 1 to 7, wherein the non-small cell lung cancer is squamous cell carcinoma.
10. The composition or medicament or treatment of any of embodiment 1 to 9, wherein the selective FGFR inhibitor is selected from the group consisting of erdafitinib, pemigatinib, infigratinib, futibatinib, derazantinib, LY287445, Debio1347, PRN1371, alofanib, bemarituzumab, GSK3052230, and FIIN-2, or a pharmaceutically acceptable salt thereof.
11. The composition or medicament or treatment of any of embodiments 1 to 10, wherein the CDK4/6 inhibitor is Compound I, or a pharmaceutically acceptable salt thereof
12. The composition or medicament or treatment of any of embodiments 1 to 10, wherein the CDK4/6 inhibitor is Compound IA.
13. The composition or medicament or treatment of embodiment 12, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
14. The composition or medicament or treatment of any of embodiments 1 to 10, wherein the CDK4/6 inhibitor is Compound III, or a pharmaceutically acceptable salt thereof.
15. The composition or medicament or treatment of any of embodiments 1 to 14, wherein the CDK4/6 inhibitor is administered to the host at least once a day for at least 14 consecutive days or 21 consecutive days.
16. The composition or medicament or treatment of any of embodiments 1 to 14, wherein the CDK4/6 inhibitor is administered to the host at least once a day for at least 28 consecutive days.
17. The composition or medicament or treatment of any of embodiments 1 to 14, wherein the CDK4/6 inhibitor is administered to the host at least once a day for at least 35 consecutive days.
18. The composition or medicament or treatment of any of embodiments 1 to 14, wherein the CDK4/6 inhibitor is administered to the host at least once a day for at least 56 consecutive days.
19. The composition or medicament or treatment of any of embodiments 1 to 18, wherein the FGFR inhibitor is administered at least once a day for at least 14 consecutive days or 21 consecutive days.
20. The composition or medicament or treatment of any of embodiments 1 to 18, wherein the FGFR inhibitor is administered at least once a day for at least 28 consecutive days.
21. The composition or medicament or treatment of any of embodiments 1 to 18, wherein the FGFR inhibitor is administered at least once a day for at least 35 consecutive days.
22. The composition or medicament or treatment of any of embodiments 1 to 18, wherein the FGFR inhibitor is administered at least once a day for at least 56 consecutive days.
23. The composition or medicament or treatment of any of embodiments 1 to 22, wherein the host, at the time of the first administration of the CDK4/6 inhibitor, is CDK4/6 inhibitor treatment naïve.
24. The composition or medicament or treatment of any of embodiments 1 to 23, wherein the cancer, at the time of the first administration of the CDK4/6 inhibitor, has acquired resistance to one or more previously administered FGFR inhibitors.
25. The composition or medicament or treatment of any of embodiments 1 to 24, wherein the cancer, at the time of the first administration of the CDK4/6 inhibitor, has acquired a mutation rendering the cancer susceptible to developing resistance to one or more FGFR inhibitors.
26. A composition or medicament for use in or treatment for reducing the development of acquired resistance to the inhibitory effects of a fibroblast growth factor receptor inhibitor in a host with a non-small cell lung cancer having an FGFR aberration, wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is selected from Compound I-VI, or a pharmaceutically acceptable salt thereof.
27. The composition or medicament or treatment of embodiment 26, wherein FGFR aberration is a result of an FGFR mutation, FGFR overexpression or amplification, or FGFR translocation or fusion.
28. The composition or medicament or medicament or treatment of any of embodiments 26 to 27, wherein the FGFR aberration is an FGFR overexpression or amplification.
29. The composition or medicament or treatment of any of embodiments 26 to 28, wherein the non-small cell lung cancer is large cell lung carcinoma.
30. The composition or medicament or treatment of any of embodiments 26 to 28, wherein the non-small cell lung cancer is squamous cell carcinoma.
31. The composition or medicament or treatment of any of embodiment 26 to 30, wherein the selective FGFR inhibitor is selected from the group consisting of erdafitinib, pemigatinib, infigratinib, AZD4547, futibatinib, derazantinib, LY287445, Debio1347, PRN1371, alofanib, bemarituzumab, and FIIN-2, or a pharmaceutically acceptable salt thereof.
32. The composition or medicament or treatment of any of embodiments 26 to 31, wherein the CDK4/6 inhibitor is Compound I, or a pharmaceutically acceptable salt thereof.
33. The composition or medicament or treatment of any of embodiments 26 to 31, wherein the CDK4/6 inhibitor is Compound IA.
34. The composition or medicament or treatment of embodiment 33, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
35. The composition or medicament or treatment of any of embodiments 26 to 31, wherein the CDK4/6 inhibitor is Compound III, or a pharmaceutically acceptable salt thereof.
36. The composition or medicament or treatment of any of embodiments 26 to 35, wherein the CDK4/6 inhibitor is administered to the host at least once a day for at least 14 or at least 21 consecutive days.
37. The composition or medicament or treatment of any of embodiments 26 to 35, wherein the CDK4/6 inhibitor is administered to the host at least once a day for at least 28 consecutive days.
38. The composition or medicament or treatment of any of embodiments 26 to 35, wherein the CDK4/6 inhibitor is administered to the host at least once a day for at least 35 consecutive days.
39. The composition or medicament or treatment of any of embodiments 26 to 35, wherein the CDK4/6 inhibitor is administered to the host at least once a day for at least 56 consecutive days.
40. The composition or medicament or treatment of any of embodiments 26 to 39, wherein the FGFR inhibitor is administered at least once a day for at least 14 or at least 21 consecutive days.
41. The composition or medicament or treatment of any of embodiments 26 to 39, wherein the FGFR inhibitor is administered at least once a day for at least 28 consecutive days.
42. The composition or medicament or treatment of any of embodiments 26 to 39, wherein the FGFR inhibitor is administered at least once a day for at least 35 consecutive days.
43. The composition or medicament or treatment of any of embodiments 26 to 39, wherein the FGFR inhibitor is administered at least once a day for at least 56 consecutive days.
44. The composition or medicament or treatment of any of embodiments 26 to 43, wherein the host, at the time of the first administration of the CDK4/6 inhibitor, is CDK4/6 inhibitor treatment naïve.
45. The composition or medicament or treatment of any of embodiments 26 to 44, wherein the cancer, at the time of the first administration of the CDK4/6 inhibitor, has acquired resistance to one or more previously administered FGFR inhibitors.
46. The composition or medicament or treatment of any of embodiments 26 to 45, wherein the cancer, at the time of the first administration of the CDK4/6 inhibitor, has acquired a mutation rendering the cancer susceptible to developing resistance to one or more FGFR inhibitors.
47. A composition or medicament for use in, or treatment for, treating a host with a gastric adenocarcinoma having a dysregulated fibroblast growth factor receptor (FGFR) signaling pathway caused by an FGFR2 aberration, wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is selected from Compound I-VI, or a pharmaceutically acceptable salt thereof.
48. The composition or medicament or treatment of embodiment 47, wherein the FGFR2 aberration is a result of an FGFR2 mutation, FGFR2 overexpression or amplification, or FGFR2 translocation or fusion.
49. The composition or medicament or treatment of embodiment 47, wherein the FGFR2 aberration is an FGFR2 overexpression or amplification.
50. The composition or medicament or treatment of any of embodiments 47 to 49, wherein the selective FGFR inhibitor is selected from the group consisting of erdafitinib, pemigatinib, infigratinib, AZD4547, futibatinib, derazantinib, LY287445, Debio1347, PRN1371, alofanib, bemarituzumab, and FIIN-2, or a pharmaceutically acceptable salt thereof.
51. The composition or medicament or treatment of any of embodiments 47 to 50, wherein the CDK4/6 inhibitor is Compound I, or a pharmaceutically acceptable salt thereof
52. The composition or medicament or treatment of any of embodiments 47 to 50, wherein the CDK4/6 inhibitor is Compound IA.
53. The composition or medicament or treatment of embodiment 52, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
54. The composition or medicament or treatment of any of embodiments 47 to 50, wherein the CDK4/6 inhibitor is Compound III, or a pharmaceutically acceptable salt thereof.
55. The composition or medicament or treatment of any of embodiments 47 to 54, wherein the CDK4/6 inhibitor is administered to the host at least once a day for at least 14 or at least 21 consecutive days.
56. The composition or medicament or treatment of any of embodiments 47 to 54, wherein the CDK4/6 inhibitor is administered to the host at least once a day for at least 28 consecutive days.
57. The composition or medicament or treatment of any of embodiments 47 to 54, wherein the CDK4/6 inhibitor is administered to the host at least once a day for at least 35 consecutive days.
58. The composition or medicament or treatment of any of embodiments 47 to 54, wherein the CDK4/6 inhibitor is administered to the host at least once a day for at least 56 consecutive days.
59. The composition or medicament or treatment of any of embodiments 47 to 58, wherein the FGFR inhibitor is administered at least once a day for at least 14 or at least 21 consecutive days.
60. The composition or medicament or treatment of any of embodiments 47 to 58, wherein the FGFR inhibitor is administered at least once a day for at least 28 consecutive days.
61. The composition or medicament or treatment of any of embodiments 47 to 58, wherein the FGFR inhibitor is administered at least once a day for at least 35 consecutive days.
62. The composition or medicament or treatment of any of embodiments 47 to 58, wherein the FGFR inhibitor is administered at least once a day for at least 56 consecutive days.
63. The composition or medicament or treatment of any of embodiments 47 to 62, wherein the host, at the time of the first administration of the CDK4/6 inhibitor, is CDK4/6 inhibitor treatment naïve.
64. The composition or medicament or treatment of any of embodiments 47 to 63, wherein the cancer, at the time of the first administration of the CDK4/6 inhibitor, has acquired resistance to one or more previously administered FGFR inhibitors.
65. The composition or medicament or treatment of any of embodiments 47 to 64, wherein the cancer, at the time of the first administration of the CDK4/6 inhibitor, has acquired a mutation rendering the cancer susceptible to developing resistance to one or more FGFR inhibitors.
66. A composition or medicament for use in, or treatment for, reducing the development of acquired resistance to the inhibitory effects of a fibroblast growth factor receptor inhibitor in a host with gastric adenocarcinoma having an FGFR aberration, wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is selected from Compound I-VI, or a pharmaceutically acceptable salt thereof.
67. The composition or medicament or treatment of embodiment 66, wherein FGFR aberration is a result of an FGFR mutation, FGFR overexpression or amplification, or FGFR translocation or fusion.
68. The composition or medicament or treatment of any of embodiments 66 to 67, wherein the FGFR aberration is an FGFR overexpression or amplification.
69. The composition or medicament or treatment of any of embodiment 66 to 68, wherein the selective FGFR inhibitor is selected from the group consisting of erdafitinib, pemigatinib, infigratinib, futibatinib, derazantinib, LY287445, Debio1347, PRN1371, alofanib, bemarituzumab, and FIIN-2.
70. The composition or medicament or treatment of any of embodiments 66 to 69, wherein the CDK4/6 inhibitor is Compound I, or a pharmaceutically acceptable salt thereof.
71. The composition or medicament or treatment of any of embodiments 66 to 69, wherein the CDK4/6 inhibitor is Compound IA.
72. The composition or medicament or treatment of embodiment 71, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
73. The composition or medicament or treatment of any of embodiments 66 to 69, wherein the CDK4/6 inhibitor Compound III, or a pharmaceutically acceptable salt thereof
74. The composition or medicament or treatment of any of embodiments 66 to 73, wherein the CDK4/6 inhibitor is administered to the host at least once a day for at least 14 or at least 21 consecutive days.
75. The composition or medicament or treatment of any of embodiments 66 to 73, wherein the CDK4/6 inhibitor is administered to the host at least once a day for at least 28 consecutive days.
76. The composition or medicament or treatment of any of embodiments 66 to 73, wherein the CDK4/6 inhibitor is administered to the host at least once a day for at least 35 consecutive days.
77. The composition or medicament or treatment of any of embodiments 66 to 73, wherein the CDK4/6 inhibitor is administered to the host at least once a day for at least 56 consecutive days.
78. The composition or medicament or treatment of any of embodiments 66 to 77, wherein the FGFR inhibitor is administered at least once a day for at least 14 or at least 21 consecutive days.
79. The composition or medicament or treatment of any of embodiments 66 to 77, wherein the FGFR inhibitor is administered at least once a day for at least 28 consecutive days.
80. The composition or medicament or treatment of any of embodiments 66 to 77, wherein the FGFR inhibitor is administered at least once a day for at least 35 consecutive days.
81. The composition or medicament or treatment of any of embodiments 66 to 77, wherein the FGFR inhibitor is administered at least once a day for at least 56 consecutive days.
82. The composition or medicament or treatment of any of embodiments 66 to 81, wherein the host, at the time of the first administration of the CDK4/6 inhibitor, is CDK4/6 inhibitor treatment naïve.
83. The composition or medicament or treatment of any of embodiments 66 to 82, wherein the cancer, at the time of the first administration of the CDK4/6 inhibitor, has acquired resistance to one or more previously administered FGFR inhibitors.
84. The composition or medicament or treatment of any of embodiments 66 to 83, wherein the cancer, at the time of the first administration of the CDK4/6 inhibitor, has acquired a mutation rendering the cancer susceptible to developing resistance to one or more FGFR inhibitors.
85. A composition or medicament for use in, or treatment for, treating a host with a dysregulated fibroblast growth factor receptor (FGFR) signaling pathway caused by an FGFR1 amplification wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is selected from Compound I-VI, or a pharmaceutically acceptable salt thereof, and wherein the cancer is selected from the group consisting of non-small cell lung cancer, small cell lung cancer, triple negative breast cancer, osteosarcoma, and HR+, HER2-amplified breast cancer, pilocytic astrocytoma, and glioblastoma.
86. The composition or medicament or treatment of any of embodiment 85, wherein the selective FGFR inhibitor is selected from the group consisting of erdafitinib, pemigatinib, infigratinib, AZD4547, futibatinib, derazantinib, Debio1347, PRN1371, FIIN, 2, GSK3052230, and PD173074.
87. The composition or medicament or treatment of any of embodiments 85 to 86, wherein the CDK4/6 inhibitor is Compound I, or a pharmaceutically acceptable salt thereof.
88. The composition or medicament or treatment of any of embodiments 85 to 86, wherein the CDK4/6 inhibitor is Compound IA.
89. The composition or medicament or treatment of embodiment 88, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
90. The composition or medicament or treatment of any of embodiments 85 to 86, wherein the CDK4/6 inhibitor Compound III, or a pharmaceutically acceptable salt thereof
91. The composition or medicament or treatment of any of embodiments 85 to 90, wherein the CDK4/6 inhibitor is administered to the host at least once a day for at least 21 consecutive days.
92. The composition or medicament or treatment of any of embodiments 85 to 90, wherein the CDK4/6 inhibitor is administered to the host at least once a day for at least 28 consecutive days.
93. The composition or medicament or treatment of any of embodiments 85 to 90, wherein the CDK4/6 inhibitor is administered to the host at least once a day for at least 35 consecutive days.
94. The composition or medicament or treatment of any of embodiments 85 to 90, wherein the CDK4/6 inhibitor is administered to the host at least once a day for at least 56 consecutive days.
95. The composition or medicament or treatment of any of embodiments 85 to 94, wherein the FGFR inhibitor is administered at least once a day for at least 14 or at least 21 consecutive days.
96. The composition or medicament or treatment of any of embodiments 85 to 94, wherein the FGFR inhibitor is administered at least once a day for at least 28 consecutive days.
97. The composition or medicament or treatment of any of embodiments 85 to 94, wherein the FGFR inhibitor is administered at least once a day for at least 35 consecutive days.
98. The composition or medicament or treatment of any of embodiments 85 to 94, wherein the FGFR inhibitor is administered at least once a day for at least 56 consecutive days.
99. The composition or medicament or treatment of any of embodiments 85 to 98, wherein the host, at the time of the first administration of the CDK4/6 inhibitor, is CDK4/6 inhibitor treatment naïve.
100. The composition or medicament or treatment of any of embodiments 85 to 99, wherein the cancer, at the time of the first administration of the CDK4/6 inhibitor, has acquired resistance to one or more previously administered FGFR inhibitors.
101. The composition or medicament or treatment of any of embodiments 85 to 100, wherein the cancer, at the time of the first administration of the CDK4/6 inhibitor, has acquired a mutation rendering the cancer susceptible to developing resistance to one or more FGFR inhibitors.
102. A composition or medicament for use in, or treatment for, treating a host with a dysregulated fibroblast growth factor receptor (FGFR) signaling pathway caused by an FGFR2 aberration, wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is selected from Compound I-VI, or a pharmaceutically acceptable salt thereof, and wherein the cancer is selected from the group consisting of ER+, HER2-amplified breast cancer, endometrial cancer, non-small cell lung cancer, gastric cancer, intrahepatic cholangiocarcinoma, and thyroid cancer.
103. The composition or medicament or treatment of embodiment 102, wherein the FGFR2 aberration is selected from the group consisting of an FGFR2 amplification, an FGFR2 mutation, and an FGFR2 translocation.
104. The composition or medicament or treatment of any of embodiment 102 to 103, wherein the selective FGFR inhibitor is selected from the group consisting of erdafitinib, pemigatinib, infigratinib, futibatinib, derazantinib, LY287445, Debio1347, PRN1371, alofanib, bemarituzumab, and FIIN-2.
105. The composition or medicament or treatment of any of embodiments 102 to 104, wherein the CDK4/6 inhibitor is Compound I, or a pharmaceutically acceptable salt thereof.
106. The composition or medicament or treatment of any of embodiments 102 to 104, wherein the CDK4/6 inhibitor is Compound IA.
107. The composition or medicament or treatment of embodiment 106, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
108. The composition or medicament or treatment of any of embodiments 102 to 104, wherein the CDK4/6 inhibitor is Compound III, or a pharmaceutically acceptable salt thereof
109. The composition or medicament or treatment of any of embodiments 102 to 108, wherein the CDK4/6 inhibitor is administered to the host at least once a day for at least 14 or at least 21 consecutive days.
110. The composition or medicament or treatment of any of embodiments 102 to 108, wherein the CDK4/6 inhibitor is administered to the host at least once a day for at least 28 consecutive days.
111. The composition or medicament or treatment of any of embodiments 102 to 108, wherein the CDK4/6 inhibitor is administered to the host at least once a day for at least 35 consecutive days.
112. The composition or medicament or treatment of any of embodiments 102 to 108, wherein the CDK4/6 inhibitor is administered to the host at least once a day for at least 56 consecutive days.
113. The composition or medicament or treatment of any of embodiments 102 to 112, wherein the FGFR inhibitor is administered at least once a day for at least 21 consecutive days.
114. The composition or medicament or treatment of any of embodiments 102 to 112, wherein the FGFR inhibitor is administered at least once a day for at least 28 consecutive days.
115. The composition or medicament or treatment of any of embodiments 102 to 112, wherein the FGFR inhibitor is administered at least once a day for at least 35 consecutive days.
116. The composition or medicament or treatment of any of embodiments 102 to 112, wherein the FGFR inhibitor is administered at least once a day for at least 56 consecutive days.
117. The composition or medicament or treatment of any of embodiments 102 to 116, wherein the host, at the time of the first administration of the CDK4/6 inhibitor, is CDK4/6 inhibitor treatment naïve.
118. The composition or medicament or treatment of any of embodiments 102 to 117, wherein the cancer, at the time of the first administration of the CDK4/6 inhibitor, has acquired resistance to one or more previously administered FGFR inhibitors.
119. The composition or medicament or treatment of any of embodiments 102 to 118, wherein the cancer, at the time of the first administration of the CDK4/6 inhibitor, has acquired a mutation rendering the cancer susceptible to developing resistance to one or more FGFR inhibitors.
120. A composition or medicament for use in, or treatment for, treating a host with a dysregulated fibroblast growth factor receptor (FGFR) signaling pathway caused by an FGFR3 aberration wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is selected from Compound I-VI, or a pharmaceutically acceptable salt thereof, and wherein the cancer is selected from the group consisting of glioblastoma, non-small cell lung cancer, cervical cancer, and multiple myeloma.
121. The composition or medicament or treatment of embodiment 120, wherein the FGFR3 aberration is selected from the group consisting of an FGFR3 amplification, an FGFR3 mutation, and an FGFR3 translocation.
122. The composition or medicament or treatment of any of embodiment 120 to 121, wherein the selective FGFR inhibitor is selected from the group consisting of erdafitinib, pemigatinib, infigratinib, futibatinib, derazantinib, LY287445, Debio1347, PRN1371, MGFR1877S, vofatamab, and FIIN-2.
123. The composition or medicament or treatment of any of embodiments 120 to 122, wherein the CDK4/6 inhibitor is Compound I, or a pharmaceutically acceptable salt thereof
124. The composition or medicament or treatment of any of embodiments 120 to 122, wherein the CDK4/6 inhibitor is Compound IA.
125. The composition or medicament or treatment of embodiment 124, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
126. The composition or medicament or treatment of any of embodiments 120 to 125, wherein the CDK4/6 inhibitor is Compound III, or a pharmaceutically acceptable salt thereof
127. The composition or medicament or treatment of any of embodiments 120 to 126, wherein the CDK4/6 inhibitor is administered to the host at least once a day for at least 14 or at least 21 consecutive days.
128. The composition or medicament or treatment of any of embodiments 120 to 126, wherein the CDK4/6 inhibitor is administered to the host at least once a day for at least 28 consecutive days.
129. The composition or medicament or treatment of any of embodiments 120 to 126, wherein the CDK4/6 inhibitor is administered to the host at least once a day for at least 35 consecutive days.
130. The composition or medicament or treatment of any of embodiments 120 to 126, wherein the CDK4/6 inhibitor is administered to the host at least once a day for at least 56 consecutive days.
131. The composition or medicament or treatment of any of embodiments 120 to 130, wherein the FGFR inhibitor is administered at least once a day for at least 14 or at least 21 consecutive days.
132. The composition or medicament or treatment of any of embodiments 120 to 130, wherein the FGFR inhibitor is administered at least once a day for at least 28 consecutive days.
133. The composition or medicament or treatment of any of embodiments 120 to 130, wherein the FGFR inhibitor is administered at least once a day for at least 35 consecutive days.
134. The composition or medicament or treatment of any of embodiments 120 to 130, wherein the FGFR inhibitor is administered at least once a day for at least 56 consecutive days.
135. The composition or medicament or treatment of any of embodiments 120 to 134, wherein the host, at the time of the first administration of the CDK4/6 inhibitor, is CDK4/6 inhibitor treatment naïve.
136. The composition or medicament or treatment of any of embodiments 120 to 135, wherein the cancer, at the time of the first administration of the CDK4/6 inhibitor, has acquired resistance to one or more previously administered FGFR inhibitors.
137. The composition or medicament or treatment of any of embodiments 120 to 136, wherein the cancer, at the time of the first administration of the CDK4/6 inhibitor, has acquired a mutation rendering the cancer susceptible to developing resistance to one or more FGFR inhibitors.
138. A composition or medicament for use in, or treatment for, treating a host with a dysregulated fibroblast growth factor receptor (FGFR) signaling pathway caused by an FGFR4 or FGF aberration, wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is Compound I-VI, or a pharmaceutically acceptable salt thereof, and wherein the cancer is selected from the group consisting of hepatocellular carcinoma, rhabdomyosarcoma, endometrial cancer, ER+, HER2-amplified breast cancer, and ovarian cancer.
139. The composition or medicament or treatment of embodiment 138, wherein the FGFR4 aberration is selected from the group consisting of an FGFR4 amplification, an FGFR4 mutation, and an FGFR4 translocation.
140. The composition or medicament or treatment of any of embodiment 138 to 139, wherein the selective FGFR inhibitor is selected from the group consisting of infigratinib, futibatinib, derazantinib, LY287445, INCB062079, BLU9931, H3-6527, fisogatinib, roblitinib, Debio1347, PRN1371, and FIIN-2.
141. The composition or medicament or treatment of any of embodiments 138 to 140, wherein the CDK4/6 inhibitor is Compound I, or a pharmaceutically acceptable salt thereof
142. The composition or medicament or treatment of any of embodiments 138 to 140, wherein the CDK4/6 inhibitor is Compound IA.
143. The composition or medicament or treatment of embodiment 142, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
144. The composition or medicament or treatment of any of embodiments 138 to 143, wherein the CDK4/6 inhibitor is Compound III, or a pharmaceutically acceptable salt thereof
145. The composition or medicament or treatment of any of embodiments 138 to 144, wherein the CDK4/6 inhibitor is administered to the host at least once a day for at least 14 or at least 21 consecutive days.
146. The composition or medicament or treatment of any of embodiments 138 to 144, wherein the CDK4/6 inhibitor is administered to the host at least once a day for at least 28 consecutive days.
147. The composition or medicament or treatment of any of embodiments 138 to 144, wherein the CDK4/6 inhibitor is administered to the host at least once a day for at least 35 consecutive days.
148. The composition or medicament or treatment of any of embodiments 138 to 144, wherein the CDK4/6 inhibitor is administered to the host at least once a day for at least 56 consecutive days.
149. The composition or medicament or treatment of any of embodiments 138 to 148, wherein the FGFR inhibitor is administered at least once a day for at least 14 or at least 21 consecutive days.
150. The composition or medicament or treatment of any of embodiments 138 to 148, wherein the FGFR inhibitor is administered at least once a day for at least 28 consecutive days.
151. The composition or medicament or treatment of any of embodiments 138 to 148, wherein the FGFR inhibitor is administered at least once a day for at least 35 consecutive days.
152. The composition or medicament or treatment of any of embodiments 138 to 151, wherein the FGFR inhibitor is administered at least once a day for at least 56 consecutive days.
153. The composition or medicament or treatment of any of embodiments 138 to 152, wherein the host, at the time of the first administration of the CDK4/6 inhibitor, is CDK4/6 inhibitor treatment naïve.
154. The composition or medicament or treatment of any of embodiments 138 to 153, wherein the cancer, at the time of the first administration of the CDK4/6 inhibitor, has acquired resistance to one or more previously administered FGFR inhibitors.
155. The composition or medicament or treatment of any of embodiments 138 to 154, wherein the cancer, at the time of the first administration of the CDK4/6 inhibitor, has acquired a mutation rendering the cancer susceptible to developing resistance to one or more FGFR inhibitors.
156. The composition or medicament or treatment of any of embodiments 1 to 155, wherein the cancer is metastatic.
157. The composition or medicament or treatment of any of embodiments 1 to 156, wherein the cancer to be treated does not harbor a mutation mutually exclusive to a FGFR aberration, for example, a KRAS or BRAF mutation.
158. The composition or medicament or treatment of any of embodiments to 1 to 157, wherein the cancer has progressed during or following at least one line of prior chemotherapy, for example a platinum-containing chemotherapy such as cisplatin, carboplatin, oxaliplatin, nedaplatin, triplatin tetranitrate, phenanthriplatin, picoplatin, or satraplatin.
159. The composition or medicament or treatment of any of embodiments 1 to 157, wherein the host is a human.
160. A composition or medicament for use in, or treatment for, treating a host with a cancer having a dysregulated fibroblast growth factor receptor (FGFR) signaling pathway caused by an FGFR or FGF aberration wherein the treatment comprises administering to the host an effective amount of a short acting CDK4/6 inhibitor, and administering to the host an effective amount of a selective fibroblast growth factor receptor (FGFR) inhibitor, wherein the CDK4/6 inhibitor is selected from Compound I-VI or a pharmaceutically acceptable salt thereof.
161. The composition or medicament or treatment of embodiment 160, wherein the FGFR or FGF aberration is a result of a mutation, overexpression or amplification, or translocation or fusion.
162. The composition or medicament or treatment of any of embodiment 160 to 162, wherein the selective FGFR inhibitor is selected from the group consisting of erdafitinib, infigratinib, pemigatinib, AZD4547, futibatinib (TAS-120), derazantinib, roblitinib, LY287445, INCB062079, BLU9931, PRN1371, FIIN-2, PD173074, H3B-6527, fisogatinib, alofanib, bemarituzumab, vofatamab, MGFR1877S, and Debio1347, or a pharmaceutically acceptable salt thereof
163. The composition or medicament or treatment of any of embodiments 160 to 162, wherein the CDK4/6 inhibitor is Compound I, or a pharmaceutically acceptable salt thereof.
164. The composition or medicament or treatment of any of embodiments 160 to 162, wherein the CDK4/6 inhibitor is Compound IA.
165. The composition or medicament or treatment of embodiment 164, wherein the CDK4/6 inhibitor is Compound IA, Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.
166. The composition or medicament or treatment of any of embodiments 160 to 165, wherein the CDK4/6 inhibitor is Compound III, or a pharmaceutically acceptable salt thereof.
167. The composition or medicament or treatment of any of embodiments 160 to 166, wherein the CDK4/6 inhibitor is administered to the host at least once a day for at least 14 consecutive days or 21 consecutive days.
168. The composition or medicament or treatment of any of embodiments 160 to 166, wherein the CDK4/6 inhibitor is administered to the host at least once a day for at least 28 consecutive days.
169. The composition or medicament or treatment of any of embodiments 160 to 166, wherein the CDK4/6 inhibitor is administered to the host at least once a day for at least 35 consecutive days.
170. The composition or medicament or treatment of any of embodiments 160 to 166, wherein the CDK4/6 inhibitor is administered to the host at least once a day for at least 56 consecutive days.
171. The composition or medicament or treatment of any of embodiments 160 to 170, wherein the FGFR inhibitor is administered at least once a day for at least 14 consecutive days or 21 consecutive days.
172. The composition or medicament or treatment of any of embodiments 160 to 170, wherein the FGFR inhibitor is administered at least once a day for at least 28 consecutive days.
173. The composition or medicament or treatment of any of embodiments 160 to 170, wherein the FGFR inhibitor is administered at least once a day for at least 35 consecutive days.
174. The composition or medicament or treatment of any of embodiments 160 to 170, wherein the FGFR inhibitor is administered at least once a day for at least 56 consecutive days.
175. The composition or medicament or treatment of any of embodiments 160 to 174, wherein the host, at the time of the first administration of the CDK4/6 inhibitor, is CDK4/6 inhibitor treatment naïve.
176. The composition or medicament or treatment of any of embodiments 160 to 175, wherein the cancer, at the time of the first administration of the CDK4/6 inhibitor, has acquired resistance to one or more previously administered FGFR inhibitors.
177. The composition or medicament or treatment of any of embodiments 160 to 176, wherein the cancer, at the time of the first administration of the CDK4/6 inhibitor, has acquired a mutation rendering the cancer susceptible to developing resistance to one or more FGFR inhibitors.
178. The composition or medicament or treatment of any of embodiments 1 to 177, wherein the host is administered an additional anti-cancer agent.
179. The composition or medicament or treatment of embodiment 178, wherein the additional anti-cancer agent is a checkpoint inhibitor, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, or other inhibitor.
180. The composition or medicament or treatment of any of embodiment 1 to 179, wherein the cancer is CDK4/6 replication dependent.

### EXAMPLES

### Example 1 Cell Proliferation and Viability Assay

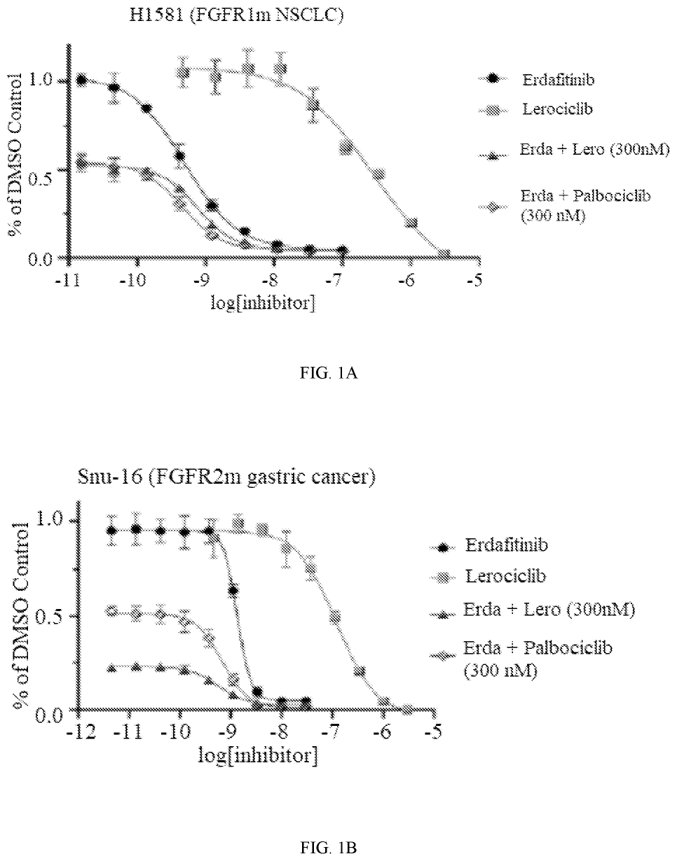

Cell viability was conducted using the CellTiter-Glo® Luminescent Cell Viability Assay. The assay was conducted with either a single agent (erdafitinib or lerociclib) or with a pair-wise combination of targeted agents (erdafitinib+lerociclib or erdafitinib+palbociclib) in three separate FGFR mutant cell lines—H1581 (FGFR1 amplified NSCLC large cell carcinoma), Snu-16 (FGFR2 amplified gastric adenocarcinoma) and RT4 (FGFR3m bladder cancer—FGFR3-TACC3 fusion). Briefly, 1×10³ cells were seeded into 96-well plates and allowed to adhere overnight. The following day, cells were treated with either single or double agents (with equimolar ratio for combinations). After 120-144 hours, 0.1 mg/mL of resazurin salt dye (Sigma Aldrich) or 20% of the manufacturer's recommended volume of Cell Titer Glo (Promega) was added. The optical density (OD) of each well was measured at 562 nm (reference wavelength: 650 nm) with a SpectraMax 250 (Molecular Devices) or EnVision (Perkin Elmer) microplate reader. Non-linear regression and sigmoidal dose-response curves were used to calculate the half maximal inhibitory concentration (IC50) with GraphPad Prism4 software. Viabilities were expressed as a percentage of the untreated controls. Results for the H1581 (FGFR1m NSCLC) cells are shown in FIG. 1A. Results for the Snu-16 (FGFR2m gastric cancer) cells are shown in FIG. 1B. Results for the RT4 (FGFR3m bladder cancer) cells are shown in FIG. 1C. The results indicate that the combination of lerociclib (Compound I) and erdafitinib consistently and synergistically inhibited FGFR mutant cell lines compared to either compound alone. In addition, lerociclib and erdafitinib was shown to be more efficacious than the combination of erdafitinib and palbociclib, another selective CDK4/6 inhibitor, at lower concentrations in, for example FGFR2m gastric cancer cell lines and FGFR3m bladder cancer call lines.

### Example 2 Measuring Impact of Lerociclib and Erdafitinib on Acquired Resistance

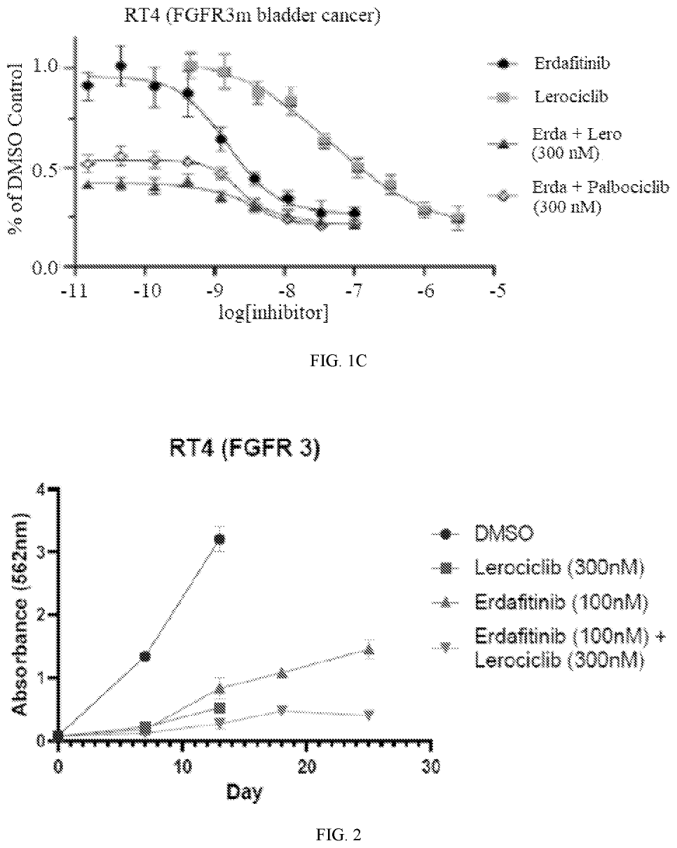

For colony formation assays, RT4 (FGFR3m bladder cancer) cells were seeded at a density of 10,000 cells/well on 6-well plates in Waymouth's media containing 10% (v/v) fetal bovine serum. The assay was conducted with either a single agent (erdafitinib or lerociclib) or with a pair-wise combination of erdafitinib (100 nM)+lerociclib (300 nM). After treatment, plates were pulled and cells were stained with Crystal Violet (Merck Millipore, Darmstadt, Germany). The optical density (OD) of each well was measured at 562 nm (reference wavelength: 650 nm) with a SpectraMax 250 (Molecular Devices) or EnVision (Perkin Elmer) microplate reader. Outgrowth was measured with GraphPad Prism4 software. Results are shown in FIG. 2. As shown, the addition of lerociclib (Compound I) drastically improved cell sensitivity over time compared to erdafitinib treatment alone, indicating a suppression of the development of FGFR inhibitor resistance in FGFRm cell lines.

### Example 3. Conversion of Compound 1 to its HCl Counterpart, Compound 1A

A representative synthesis of Compound 1A is provided:

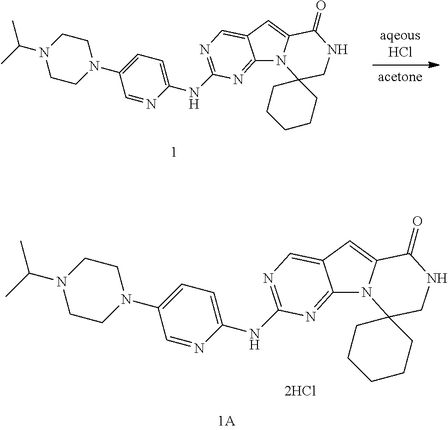 [SMILES: CC(C)N1CCN(c2ccc(Nc3ncc4cc5n(c4n3)C3(CCCCC3)CNC5=O)nc2)CC1 ; CC(C)N1CCN(c2ccc(Nc3ncc4cc5n(c4n3)C3(CCCCC3)CNC5=O)nc2)CC1]

Compound 1 (0.9 kg. 1.9 moles, 1 eq) was charged to a 22 L flask and dissolved in aqueous, 2 M hydrochloric acid solution (3.78 L). The solution was heated to 50±5° C., stirred for 30 minutes, and the resulting mixture filtered over Celite (alternatively the solution may be filtered through a 0.45 micron in-line filter) to afford Compound 1A. The flask was rinsed with 0.1 M hydrochloric acid solution to collect any additional Compound 1A. Compound 1A was then heated to 50±5° C. while acetone (6.44 L) was slowly added. The solution was stirred at 50±5° C. for 30 minutes, the temperature was decreased to 20±5° C., and stirring continued for 2 hours. The solids were collected by filtration, washed with acetone, and dried to afford 820.90 g of Compound 1A (82.1% yield). In some embodiments instead of acetone, ethanol is used.

### Example 4: Recrystallization Procedures to Produce Form B from Compound 1

Recrystallization Process 1: Compound 1 was charged to an appropriately sized flask or reactor, dissolved in aqueous hydrochloric acid solution and heated to at least 55±10° C. The solution was stirred for about 45 minutes and the resulting mixture was filtered through an in-line filter. Acetone was added at 55±10° C. over the course of an hour and the solution was stirred for about an additional hour. The temperature was decreased to about 25±5° C., and the solution was stirred for at least 2 hours. The solids were collected by filtration, washed with acetone, and dried to afford Compound 1A form B.

### Example 5. XRPD Analysis of Compound 1A Morphic Form B

The XRPD pattern of Form B was collected with a PANalytical X'Pert PRO MPD diffractometer using an incident beam of Cu radiation produced using an Optix long, fine-focus source. An elliptically graded multilayer mirror was used to focus Cu Kα X-rays through the specimens and onto the detector. Prior to the analysis, a silicon specimen (NIST SRM 640e) was analyzed to verify the observed position of the Si 111 peak is consistent with the NIST-certified position. The sample was sandwiched between 3-μm-thick films and analyzed in transmission geometry. A beamstop, short anti-scatter extension and an anti-scatter knife edge were used to minimize the background generated by air. Soller slits for the incident and diffracted beams were used to minimize broadening from axial divergence. The diffraction patterns were collected using a scanning position-sensitive detector (X'Celerator) located 240 mm from the specimens and Data Collector software v. 2.2b. Data acquisition parameters for each pattern are displayed above the image in the Data section of this report including the divergence slit (DS) before the mirror.

The XRPD pattern of pure Form B along with the indexing solution is shown in FIG. 7. The pure Form B XRPD pattern exhibited sharp peaks, indicating the sample was composed of crystalline material. The allowed peak positions from the XRPD indexing solution are 6.5, 8.1, 9.4, 9.6, 10.2, 10.6, 11.2, 12.2, 12.9, 13.0, 13.3, 13.4, 14.0, 14.4, 14.6, 15.0, 15.9, 16.2, 16.4, 16.5, 16.8, 18.1, 18.4, 18.5, 18.6, 18.6, 18.9, 19.1, 19.2, 19.3, 19.4, 19.5, 19.6, 19.7, 19.8, 19.9, 20.4, 20.6, 21.3, 21.4, 21.8, 22.0, 22.2, 22.3, 22.4, 22.5, 22.8, 23.0, 23.1, 23.4, 23.8, 24.1, 24.2, 24.3, 24.4, 24.5, 24.6, 25.4, 25.6, 25.7, 25.9, 26.0, 26.1, 26.3, 26.4, 26.5, 26.6, 26.7, 26.8, 26.9, 27.2, 27.3, 27.5, 27.6, 27.7, 27.9, 28.3, 28.4, 28.5, 28.7, 28.9, 29.0, 29.1, 29.3, 29.4, 29.5, 29.6, 29.7, 29.8, 29.9, 30.0, 30.3, 30.4, 30.5, 30.6, 30.7, 30.9, 31.2, 31.5, 31.6, 31.7, 31.8, 31.9, 32.0, 32.2, 32.3, 32.4, 32.5, 32.6, 32.7, 32.8, 33.1, 33.2, 33.3, 33.6, 33.7, 33.8, 34.0, 34.1, 34.2, 34.3, 34.6, 34.7, 34.8, 35.0 35.2, 35.3, 35.5, 35.6, 35.9, 36.0, 36.2, 36.5, 36.6, 36.7, 36.8, 36.9, 37.1, 37.2, 37.3, 37.4, 37.5, 37.6, 37.7, 37.8, 37.9, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, and 40.0° 2θ.

For example, Form B's XRPD may be indexed as follows 6.47, 8.08, 9.42, 9.59, 10.18, 10.62, 11.22, 12.17, 12.91, 12.97, 13.27, 13.37, 14.03, 14.37, 14.63, 15.02, 15.93, 16.20, 16.35, 16.43, 16.47, 16.81, 18.10, 18.35, 18.41, 18.50, 18.55, 18.60, 18.91, 19.11, 19.15, 19.24, 19.34, 19.43, 19.51, 19.61, 19.65, 19.76, 19.85, 19.90, 20.44, 20.61, 21.34, 21.43, 21.84, 21.95, 22.17, 22.28, 22.30, 22.33, 22.44, 22.54, 22.76, 22.81, 22.97, 23.00, 23.11, 23.42, 23.80, 24.11, 24.22, 24.34, 24.38, 24.40, 24.48, 24.56, 24.57, 25.40, 25.56, 25.57, 25.59, 25.72, 25.74, 25.94, 25.99, 26.11, 26.28, 26.29, 26.37, 26.51, 26.58, 26.61, 26.73, 26.81, 26.92, 27.15, 27.19, 27.23, 27.31, 27.49, 27.57, 27.61, 27.71, 27.88, 27.94, 28.27, 28.41, 28.53, 28.71, 28.74 28.86, 28.94, 28.98, 29.03, 29.06, 29.08, 29.25, 29.30, 29.38, 29.51, 29.57, 29.61, 29.70, 29.73, 29.75, 29.90, 29.95, 30.31, 30.38, 30.42, 30.54, 30.55, 30.66, 30.73, 30.85, 30.87, 30.89, 31.23, 31.51, 31.55, 31.61, 31.70, 31.76, 31.77, 31.80, 31.81, 31.82, 31.82, 31.90, 31.91, 31.95, 32.17, 32.21, 32.23, 32.25, 32.36, 32.37, 32.43, 32.53, 32.54, 32.56, 32.61, 32.73, 32.80, 32.82, 33.05, 33.13, 33.17, 33.22, 33.28, 33.30, 33.60, 33.65, 33.71, 33.76, 33.77, 33.99, 34.01, 34.01, 34.05, 34.10, 34.17, 34.29, 34.55, 34.60, 34.62, 34.63, 34.68, 34.75, 34.76, 35.03, 35.16, 35.19, 35.21, 35.25, 35.31, 35.46, 35.61, 35.63, 35.85, 35.86, 35.90, 35.97, 36.19, 36.45, 36.56, 36.58, 36.67, 36.68, 36.70, 36.71, 36.77, 36.85, 36.87, 36.90, 37.09, 37.19, 37.27, 37.28, 37.29, 37.32, 37.33, 37.37, 37.38, 37.48, 37.48, 37.50, 37.51, 37.54, 37.61, 37.64, 37.65, 37.68, 37.69, 37.71, 37.74, 37.74, 37.76, 37.81, 37.83, 37.93, 37.94, 38.15, 38.19, 38.32, 38.36, 38.39, 38.46, 38.59, 38.63, 38.69, 38.76, 38.79, 38.85, 38.87, 38.88, 38.96, 38.98, 39.02, 39.05, 39.19, 39.27, 39.33, 39.36, 39.39, 39.43, 39.44, 39.53, 39.53, 39.6, 39.61, 39.70, 39.71, 39.72, 39.82, 39.87, 39.9, and 39.98° 2θ.

Observed peaks for Form B include 9.5±0.2, 18.1±0.2, 19.3±0.2, 22.4±0.2, 26.6±0.2, and 27.7±0.2, ° 2θ.

Agreement between the allowed peak positions, marked with bars, and the observed peaks indicated a consistent unit cell determination. Successful indexing of the pattern indicated that the sample was composed primarily of a single crystalline phase. Space groups consistent with the assigned extinction symbol, unit cell parameters, and derived quantities are given in Table 8.

TABLE 8
Parameters of the XRPD of Compound 1A Form B

| Bravais Type | C-centered Monoclinic |
|---|---|
| a [Å] | 27.719 |
| b [Å] | 9.796 |
| c [Å] | 22.221 |
| α [deg] | 90 |
| β [deg] | 100.16 |
| γ [deg] | 90 |
| Volume [Å³/cell] | 5,939.0 |
| Chiral contents | Not specified |
| Extinction Symbol | C 1 c 1 |
| Space Group(s) | Cc (9), C2/c (15) |

In some embodiments, Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least three 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least four 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least five 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least six 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising the 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least the 2theta value of 9.5±0.4°.

This specification has been described with reference to embodiments of the invention. However, one of ordinary skill in the art appreciates that various modification and changes can be made without departing from the scope of the invention as set forth in the claims below. Accordingly, the specification is to be regarded in an illustrative rather than a restrictive sense, and all such modifications are intended to be included within the scope of the invention.

What is claimed is:

1. A method of treating a human host with a cancer having a dysregulated fibroblast growth factor receptor (FGFR) signaling pathway caused by an FGFR1 aberration comprising administering to the host an effective amount of a short acting cyclin dependent kinase 4/6 (CDK4/6) inhibitor, and administering to the host an effective amount of a selective fibroblast growth factor receptor-tyrosine kinase inhibitor (FGFR-TKI), wherein the CDK4/6 inhibitor is 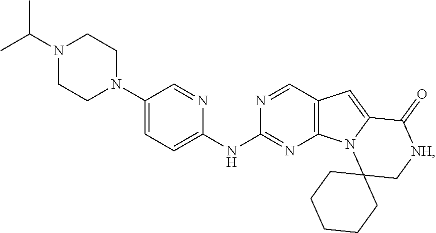 [SMILES: CC(C)N1CCN(c2ccc(Nc3ncc4cc5n(c4n3)C3(CCCCC3)CNC5=O)nc2)CC1]
or a pharmaceutically acceptable salt thereof.

2. The method of claim 1, wherein the FGFR1 aberration is an amplification or overexpression.

3. The method of claim 1, wherein the cancer is selected from the group consisting of non-small cell lung cancer, small cell lung cancer, triple negative breast cancer, osteosarcoma, pilocytic astrocytoma, and glioblastoma.

4. The method of claim 1, wherein the selective FGFR-TKI is selected from the group consisting of erdafitinib, pemigatinib, infigratinib, futibatinib, derazantinib, Debio1347, PRN1371, FIIN, 2, GSK3052230, and PD173074.

5. The method of claim 1, wherein the CDK4/6 inhibitor is 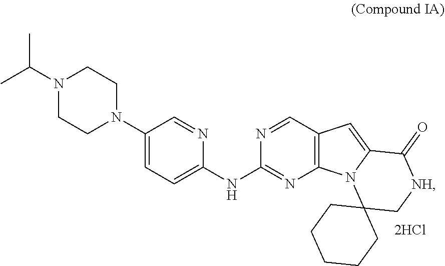 [SMILES: CC(C)N1CCN(c2ccc(Nc3ncc4cc5n(c4n3)C3(CCCCC3)CNC5=O)nc2)CC1]
Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.

6. The method of claim 1, wherein the CDK4/6 inhibitor and the FGFR-TKI are administered to the host at least once a day for at least 28 consecutive days.

7. A method of treating a human host with a cancer having a dysregulated fibroblast growth factor receptor (FGFR) signaling pathway caused by an FGFR2 aberration comprising administering to the host an effective amount of a short acting CDK4/6 inhibitor, and administering to the host an effective amount of a selective FGFR-TKI, wherein the CDK4/6 inhibitor is 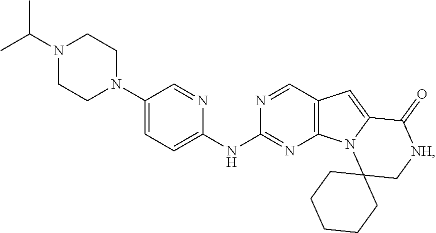 [SMILES: CC(C)N1CCN(c2ccc(Nc3ncc4cc5n(c4n3)C3(CCCCC3)CNC5=O)nc2)CC1]
or a pharmaceutically acceptable salt thereof.

8. The method of claim 7, wherein the cancer is selected from the group consisting of endometrial cancer, non-small cell lung cancer, gastric cancer, intrahepatic cholangiocarcinoma, and thyroid cancer.

9. The method of claim 7, wherein the FGFR2 aberration is selected from the group consisting of an amplification and an overexpression.

10. The method of claim 7, wherein the selective FGFR-TKI is selected from the group consisting of erdafitinib, pemigatinib, infigratinib, futibatinib, derazantinib, LY287445, Debio1347, PRN1371, alofanib, bemarituzumab, and FIIN-2.

11. The method of claim 7, wherein the CDK4/6 inhibitor is 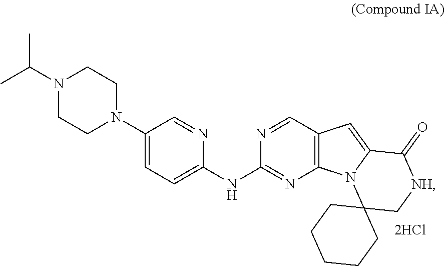 [SMILES: CC(C)N1CCN(c2ccc(Nc3ncc4cc5n(c4n3)C3(CCCCC3)CNC5=O)nc2)CC1]
Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.

12. The method of claim 7, wherein the CDK4/6 inhibitor and the FGFR-TKI are administered to the host at least once a day for at least 28 consecutive days.

13. A method of treating a human host with a cancer with a dysregulated fibroblast growth factor receptor (FGFR) signaling pathway caused by an FGFR3 aberration comprising administering to the host an effective amount of a short acting CDK4/6 inhibitor, and administering to the host an effective amount of a selective FGFR-TKI, wherein the CDK4/6 inhibitor is 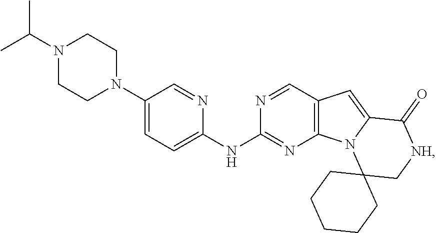 [SMILES: CC(C)N1CCN(c2ccc(Nc3ncc4cc5n(c4n3)C3(CCCCC3)CNC5=O)nc2)CC1]
or a pharmaceutically acceptable salt thereof.

14. The method of claim 13, wherein the cancer is selected from the group consisting of glioblastoma, non-small cell lung cancer, cervical cancer, and multiple myeloma.

15. The method of claim 13, wherein the FGFR3 aberration is an FGFR3 translocation or fusion.

16. The method of claim 13, wherein the selective FGFR-TKI is selected from the group consisting of erdafitinib, pemigatinib, infigratinib, futibatinib, derazantinib, LY287445, Debio1347, PRN1371, MGFR1877S, vofatamab, and FIIN-2.

17. The method of claim 13, wherein the CDK4/6 inhibitor is 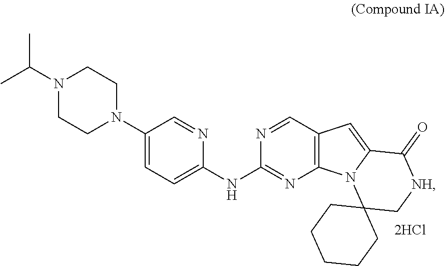 [SMILES: CC(C)N1CCN(c2ccc(Nc3ncc4cc5n(c4n3)C3(CCCCC3)CNC5=O)nc2)CC1]
Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.

18. The method of claim 13, wherein the CDK4/6 inhibitor and the FGFR-TKI are administered to the host at least once a day for at least 28 consecutive days.

19. A method of treating a human host with a cancer having a dysregulated fibroblast growth factor receptor (FGFR) signaling pathway caused by an FGFR4 aberration or FGF aberration wherein the FGFR4 aberration comprising administering to the host an effective amount of a CDK4/6 inhibitor, and administering to the host an effective amount of a selective fibroblast growth factor receptor (FGFR) inhibitor, wherein the CDK4/6 inhibitor is 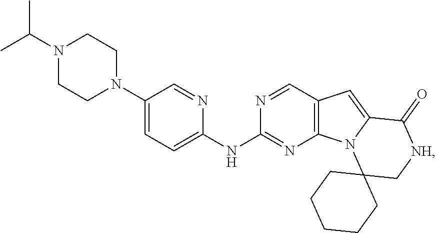 [SMILES: CC(C)N1CCN(c2ccc(Nc3ncc4cc5n(c4n3)C3(CCCCC3)CNC5=O)nc2)CC1]
or a pharmaceutically acceptable salt thereof.

20. The method of claim 19, wherein the cancer is selected from the group consisting of hepatocellular carcinoma, rhabdomyosarcoma, endometrial cancer, and ovarian cancer.

21. The method of claim 19, wherein the selective FGFR inhibitor is selected from the group consisting of infigratinib, futibatinib, derazantinib, LY287445, INCB062079, BLU9931, H3-6527, fisogatinib, roblitinib, Debio1347, PRN1371, and FIIN-2.

22. The method of claim 19, wherein the CDK4/6 inhibitor is 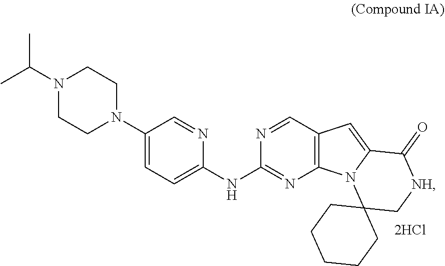 [SMILES: CC(C)N1CCN(c2ccc(Nc3ncc4cc5n(c4n3)C3(CCCCC3)CNC5=O)nc2)CC1]
Form B, wherein Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°.

23. The method of claim 19, wherein the CDK4/6 inhibitor and the FGFR-TKI are administered to the host at least once a day for at least 28 consecutive days.
